SEQUENCE LISTING (30 sequences)

SEQ ID NO: 1 - protein, 268 aa, Artificial Sequence
  scFv directed against MCP-1 (3)
EVQLLESGGGLVQPGGSLRLSCAASGFTFSNYGMHWVRQAPGKGLEWVAVISYDGSNKYYADSVKGRFTISRDNSKNTLYLQMNSLRAEDTAVYYCAKSHYYDTTSFDYWGQGTLVTVSSGGGGSGGGGSGGGGSQSVLTQPPSASGTPGQRVTISCSGSSSNIGTNPVNWYQQLPGTAPKLLIYDNNKRPSGVPDRFSGSKSGTSASLAISGLRSEDEADYYCAAWDDSLSGVVFGGGTKLTVLGEQKLISEEDLSGSAAAHHHHHH

SEQ ID NO: 2 - protein, 261 aa, Artificial Sequence
  scFv directed against Procathepsin W
EVQLLESGGGLVQPGGSLRLSCAASGFTFSSYAMSWVRQAPGKGLEWVSSMSASGGSTYYADSVKGRFTISRDNSKNTLYLQMNSLRAEDTAVYYCARDRGSYGMDVWGQGTLVTVSSGGGGSGGGGSGGGGSQSVLTQPPSASGTPGQRVTISCSGSTSNIGSYAVNWYQQLPGTAPKLLIYGNNNRPSGVPDRFSGSKSGTSASLAISGPRSEDEADYYCAAWDDSLNGGVFGGGTKLTVLGDYKDDDDKAAAHHHHHH

SEQ ID NO: 3 - protein, 267 aa, Artificial Sequence
  scFv directed against IL-4 (3)
EVQLLESGGGLVQPGGSLRLSCAASGFTFSNAWMSWVRQAPGKGLEWVSAISGSGGSTYYADSVKGRFTISRDNSKNTLYLQMNSLRAEDTAVYYCARAIAARPFDYWGQGTLVTVSSGGGGSGGGGSGGGGSQSVLTQPPSASGTPGQRVTISCTGATSNIGAGYDIHWYQQLPGTAPKLLIYSTNNRPSGVPDRFSGSKSGTSASLAISGLRSEDEADYYCAAWDDSLNGPVFGGGTKLTVLGEQKLISEEDLSGSAAAHHHHHH

SEQ ID NO: 4 - protein, 278 aa, Artificial Sequence
  scFv directed against Factor B (2)
EVQLLESGGGLVQPGGSLRLSCAASGFTFSSYSMNWVRQAPGKGLEWVAVISYDGRFIYYSDSVKGRFTISRDNSKNTLYLQMNSLRAEDTAVYYCARSYGGNLAMDVWGQGTLVTVSSGGGGSGGGGSGGGGSQSVLTQPPSASGTPGQRVTISCTGSSSNIGAGYDVHWYQQLPGTAPKLLIYDNNKRPSGVPDRFSGSNSGTSASLAISGLRSEDEADYYCAAWDDRLNGRVVFGGGTKLTVLGDYKDHDGDYKDHDIDYKDDDDKAAAHHHHHH

SEQ ID NO: 5 - protein, 274 aa, Artificial Sequence
  scFv directed against Factor B (3)
EVQLLESGGGLVQPGGSLRLSCAASGFTFSSYSMNWVRQAPGKGLEWVAVISYDGSNQYYADSVRGRFTISKDNSKNTLYLQMNSLRAEDTAVYYCAREWHYSLDVWGQGTLVTVSSGGGGSGGGGSGGGGSQSVLTQPPSASGTPGQRVTISCSGSSSNIGSNTVNWYQQLPGTAPKLLIYRNNQRPSGVPDRFSGSKSGTSASLAISGLRSEDEADYYCAAWDDSLSVRVFGGGTKLTVLGDYKDHDGDYKDHDIDYKDDDDKAAAHHHHHH

SEQ ID NO: 6 - protein, 272 aa, Artificial Sequence
  scFv directed against UPF3B (1)
EVQLLESGGGLVQPGGSLRLSCAASGFTFSDYYMTWIRQAPGKGLEWVSDISWNGSRTHYADSVKGRFTISRDNSKNTLYLQMNSLRAEDTAVYYCSSHLVYWGQGTLVTVSSGGGGSGGGGSGGGGSQSVLTQPPSASGTPGQRVTISCTGSSSNIGAGYDVHWYQQLPGTAPKLLIYDNNKRPSGVPDRFSGSKSGTSASLAISGLRSEDEADYYCQTYDSSLSGSVVFGGGTKLTVLGDYKDHDGDYKDHDIDYKDDDDKAAAHHHHHH

SEQ ID NO: 7 - protein, 274 aa, Artificial Sequence
  scFv directed against HADH2 (3) and/or GSN
EVQLLESGGGLVQPGGSLRLSCAASGFTFGSSYMSWVRQAPGKGLEWVSSISSYGYYTGYADSVKGRFTISRDNSKNTLYLQMNSLRAEDTAVYYCARSYGSWYFDYWGQGTLVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQGFVGPSTFGQGTKLEIKRLGDYKDHDGDYKDHDIDYKDDDDKAAAHHHHHH

SEQ ID NO: 8 - protein, 273 aa, Artificial Sequence
  scFv directed against PAK-7 (1)
EVQLLESGGGLVQPGGSLRLSCAASGFTFSGYSMSWVRQAPGKGLEWVSSISSSYSSTYYADSVKGRFTISRDNSKNTLYLQMNSLRAEDTAVYYCARGSFGFDYWGQGTLVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQYYYGVLPTFGQGTKLEIKRLGDYKDHDGDYKDHDIDYKDDDDKAAAHHHHHH

SEQ ID NO: 9 - protein, 276 aa, Artificial Sequence
  scFv directed against KKCC1-1 (3)
EVQLLESGGGLVQPGGSLRLSCAASGFTFYYSYMYWVRQAPGKGLEWVSAISGSGGSTYYADSVKGRFTISRDNSKNTLYLQMNSLRAEDTAVYYCARSYTPASYRFDYWGQGTLVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKLEIKRLGDYKDHDGDYKDHDIDYKDDDDKAAAHHHHHH

SEQ ID NO: 10 - protein, 273 aa, Artificial Sequence
  scFv directed against MAGI1-1 (1)
EVQLLESGGGLVQPGGSLRLSCAASGFTFSYSSMSWVRQAPGKGLEWVSGISGSGYSTYYADSVKGRFTISRDNSKNTLYLQMNSLRAEDTAVYYCARGYSGFDYWGQGTLVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQYGYATLPTFGQGTKLEIKRLGDYKDHDGDYKDHDIDYKDDDDKAAAHHHHHH

SEQ ID NO: 11 - protein, 274 aa, Artificial Sequence
  scFv directed against OTUB1-1 (2)
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARYHYYAGFDYWGQGTLVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSGSFLPTFGQGTKLEIKRLGDYKDHDGDYKDHDIDYKDDDDKAAAHHHHHH

SEQ ID NO: 12 - protein, 281 aa, Artificial Sequence
  scFv directed against FER (1)
EVQLLESGGGLVQPGGSLRLSCAASGFTFGYSYMHWVRQAPGKGLEWVSYISSYGGYTGYADSVKGRFTISRDNSKNTLYLQMNSLRAEDTAVYYCARSSVDSVVWYGGYIDYWGQGTLVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSGFNSPHTFGQGTKLEIKRLGDYKDHDGDYKDHDIDYKDDDDKAAAHHHHHH

SEQ ID NO: 13 - protein, 11 aa, Artificial Sequence
  CIMS (4) peptide sequence motif
GIVKYLYEDEG

SEQ ID NO: 14 - protein, 6 aa, Artificial Sequence
  CIMS (8) peptide sequence motif
DFAEDK

SEQ ID NO: 15 - protein, 6 aa, Artificial Sequence
  CIMS (9) peptide sequence motif
LTEFAK

SEQ ID NO: 16 - protein, 6 aa, Artificial Sequence
  CIMS (10) peptide sequence motif
TEEQLK

SEQ ID NO: 17 - protein, 6 aa, Artificial Sequence
  CIMS (13)/CIMS (14) peptide sequence motif
SSAYSR

SEQ ID NO: 18 - protein, 4 aa, Artificial Sequence
  CIMS (16) peptide sequence motif
EDFR

SEQ ID NO: 19 - protein, 6 aa, Artificial Sequence
  CIMS (25) peptide sequence motif
LSADHR

SEQ ID NO: 20 - protein, 6 aa, Artificial Sequence
  CIMS (27)/CIMS (28) peptide sequence motif
SEAHLR

SEQ ID NO: 21 - protein, 269 aa, Artificial Sequence
  CIMS (4) amino acid sequence
EVQLLESGGGLVQPGGSLRLSCAASGFTFSNYAMHWVRQAPGKGLEWVSSISNRGSRTFYADSVKGRFTISRDNSKNTLYLQMNSLRAEDTAVYYCARDHRWDPGAFDIWGQGTLVTVSSGGGGSGGGGSGGGGSQSVLTQPPSASGTPGQRVTISCTGSSSNIGADYDVHWYQQLPGTAPKLLIYGNSNRPSGVPDRFSGSKSGTSASLAISGLRSEDEADYYCAAWDDGLSGVVFGGGTKLTVLGEQKLISEEDLSGSAAAHHHHHH

SEQ ID NO: 22 - protein, 271 aa, Artificial Sequence
  CIMS (8) amino acid sequence
EVQLLESGGGLVQPGGSLRLSCAASGFTFSSYAMSWVRQAPGKGLEWVSSISSSSSYIYYADSVKGRFTISRDNSKNTLYLQMNSLRAEDTAVYYCARLFFSGGATRAAFDIWGQGTLVTVSSGGGGSGGGGSGGGGSQSVLTQPPSASGTPGQRVTISCSGGSSNIGSNTVNWYQQLPGTAPKLLIYGNSNRPSGVPDRFSGSKSGTSASLAISGLRSEDEADYYCAAWDDSLNGRVFGGGTKLTVLGEQKLISEEDLSGSAAAHHHHHH

SEQ ID NO: 23 - protein, 274 aa, Artificial Sequence
  CIMS (9) amino acid sequence
EVQLLESGGGLVQPGGSLRLSCAASGFTFSDYYMSWIRQAPGKGLEWVSSISSRSSYIYYADSVKGRFTISRDNSKNTLYLQMNSLRAEDTAVYYCAKDREYYDILTGYPSMDVWGQGTLVTVSSGGGGSGGGGSGGGGSQSVLTQPPSASGTPGQRVTISCTGSSSNIGAGYDVHWYQQLPGTAPKLLIYDNNKRPSGVPDRFSGSKSGTSASLAISGLRSEDEADYYCSAWDESLSGVVFGGGTKLTVLGEQKLISEEDLSGSAAAHHHHHH

SEQ ID NO: 24 - protein, 266 aa, Artificial Sequence
  CIMS (10) amino acid sequence
EVQLLESGGGLVQPGGSLRLSCAASGFTFSSYGMHWVRQAPGKGLEWVSAISGSGGSTYYADSVKGRFTISRDNSKNTLYLQMNSLRAEDTAVYYCARSRYGSGMDVWGQGTLVTVSSGGGGSGGGGSGGGGSQSVLTQPPSASGTPGQRVTISCSGSSSNVGVNYVYWYQQLPGTAPKLLIYSHNQRPSGVPDRFSGSKSGTSASLAISGLRSEDEADYYCAAWDDSLNGVVFGGGTKLTVLGEQKLISEEDLSGSAAAHHHHHH

SEQ ID NO: 25 - protein, 273 aa, Artificial Sequence
  CIMS (13) amino acid sequence
EVQLLESGGGLVQPGGSLRLSCAASGFTFSSYAMSWVRQAPGKGLEWVSAISGSGGRTYYTDSVRDRFTISRDNSKNTLYLQMNSLRAEDTAVYYCARDLMPVCQYCYGMDVWGQGTLVTVSSGGGGSGGGGSGGGGSQSVLTQPPSASGTPGQRVTISCTGSSSNIGAGYDVHWYQQLPGTAPKLLIYSNNQRPSGVPDRFSGSKSGTSASLAISGLRSEDEADYYCQSYDSSLNKDVVFGGGTKLTVLGEQKLISEEDLSGSAAAHHHHHH

SEQ ID NO: 26 - protein, 267 aa, Artificial Sequence
  CIMS (14) amino acid sequence
EVQLLESGGGLVQPGGSLRLSCAASGFTFSDYYMSWVRQAPGKGLEWVADIKRDGSTRYYGDSVKGRFTISRDNSKNTLYLQMNSLRAEDTAVYYCARDRLVAGLFDYWGQGTLVTVSSGGGGSGGGGSGGGGSQSVLTQPPSASGTPGQRVTISCTGSSSNIGAGYDVHWYQQLPGTAPKLLIYGNSNRPSGVPDRFSGSKSGTSASLAISGLRSEDEADYYCAAWDDSLSVLFGGGTKLTVLGEQKLISEEDLSGSAAAHHHHHH

SEQ ID NO: 27 - protein, 266 aa, Artificial Sequence
  CIMS (16) amino acid sequence
EVQLLESGGGLVQPGGSLRLSCAASGFTFDDYGMSWVRQAPGKGLEWVSAISGSGGSTYYADPVKGRFTISRDNSKNTLYLQMNSLRAEDTAVYYCARSRYGSGMDVWGQGTLVTVSSGGGGSGGGGSGGGGSQSVLTQPPSASGTPGQRVTISCSGSSSNIGSNYVYWYQQLPGTAPKLLIYKSNQRPSGVPDRFSGSKSGTSASLAISGLRSEDEADYYCAAWDDRLNAVVFGGGTKLTVLGEQKLISEEDLSGSAAAHHHHHH

SEQ ID NO: 28 - protein, 269 aa, Artificial Sequence
  CIMS (25) amino acid sequence
EVQLLESGGGLVQPGGSLRLSCAASGFTFSNYAMSWVRQAPGKGLEWVAFIRYDGSNKYYADSVKGRFTISRDNSKNTLYLQMNSLRAEDTAVYYCARDAVGGDSYVLDYWGQGTLVTVSSGGGGSGGGGSGGGGSQSVLTQPPSASGTPGQRVTISCSGSSSNIGSNAVNWYQQLPGTAPKLLIYGNSNRPSGVPDRFSGSKSGTSASLAISGLRSEDEADYYCAAWDDSLNGWVFGGGTKLTVLGEQKLISEEDLSGSAAAHHHHHH

SEQ ID NO: 29 - protein, 265 aa, Artificial Sequence
  CIMS (27) amino acid sequence
EVQLLESGGGLVQPGGSLRLSCAASGFTFTSYSMSWVRQAPGKGLEWVSAIGTGGGTYYADSVKGRFTISRDNSKNTLYLQMNSLRAEDTAVYYCARVNWNDAFDYWGQGTLVTVSSGGGGSGGGGSGGGGSQSVLTQPPSASGTPGQRVTISCSGSSSNIGNNAVNWYQQLPGTAPKLLIYRNDQRPSGVPDRFSGSKSGTSASLAISGLRSEDEADYYCSTWDDSLSGVFFGGGTKLTVLGEQKLISEEDLSGSAAAHHHHHH

SEQ ID NO: 30 - protein, 267 aa, Artificial Sequence
  CIMS (28) amino acid sequence
EVQLLESGGGLVQPGGSLRLSCAASGFTFSSYAMSWVRQAPGKGLEWVAAIWSDGSNKYYADSVKGRFTISRDNSKNTLYLQMNSLRAEDTAVYYCAKVGATDDAFDIWGQGTLVTVSSGGGGSGGGGSGGGGSQSVLTQPPSASGTPGQRVTISCSGSSSNIGSNTVNWYQQLPGTAPKLLIYGNSNRPSGVPDRFSGSKSGISASLAISGLRSEDEADYYCAAWDDSLNGPVFGGGTKLTVLGEQKLISEEDLSGSAAAHHHHHH

(19) United States
(12) Patent Application Publication
(10) Pub. No.: US 2022/0163524 A1
(43) Pub. Date: May 26, 2022

(54) METHODS, ARRAYS AND USES THEREOF FOR DIAGNOSING OR DETECTING AN AUTOIMMUNE DISEASE

(71) Applicant: Immunovia AB, Lund (SE)

(72) Inventors: ANNA ISINGER-EKSTRAND, Hoor (SE); Borje Ola Mattias Ohlsson, Lund (SE); Christer Wingren, Sodra Sandby (SE)

(21) Appl. No.: 17/442,731

(22) PCT Filed: Mar. 27, 2020
(86) PCT No.: PCT/EP2020/058767

§ 371 Date: Sep. 24, 2021

(30) Foreign Application Priority Data
Mar. 29, 2019 (GB) 1904472.6

(51) Int. Cl. G01N 33/564; C12Q 1/6883; G01N 33/58; G01N 33/60

(52) U.S. Cl. CPC G01N 33/564; C12Q 1/6883; G01N 33/582; G01N 33/60; G01N 2800/328; G01N 2800/104; G01N 2800/102; G01N 2800/101; C12Q 2600/158

(57) ABSTRACT

The present invention provides a method for diagnosing or detecting an autoimmune disease in an individual, the method comprising or consisting of the steps of (a) providing a sample obtained from an individual to be tested; and (b) measuring the presence and/or amount in the test sample of one or more biomarkers selected from the group defined in Table 1(A), Table 1(B), Table 1(C), Table 1(D) and/or Table 1(E) wherein the presence and/or amount in the sample of the one or more biomarker(s) is indicative of an autoimmune disease in the individual. The invention also provides an array and a kit suitable for use in the methods of the invention.

### FIELD OF INVENTION

The present invention provides in vitro methods for diagnosing or detecting an autoimmune disease in an individual, as well as arrays and kits for use in such methods.

### BACKGROUND

Autoimmune diseases (AID) constitute a large group of chronic and severe disorders, characterized by an abnormal response from the immune system in which healthy cells are attacked. Patients diagnosed with an autoimmune disease are faced with a life sentence, with severe side effects and increased mortality. Systemic Erythematosus Lupus (SLE), Rheumatoid Arthritis (RA), Sjögren Syndrome (SS) and Systemic Vasculitis (SV) represent four systemic autoimmune diseases (AID), which if left untreated can lead to severe and sometimes permanent physiological disability and increased morbidity (1, 2). Diagnosis at an early stage plays a crucial role for enabling proper disease monitoring and therapeutic interventions to prevent or minimize organ and tissue related damage. However, clinical diagnosis remains a challenge due to fluctuating symptoms over time, including a wide repertoire of symptoms such as fatigue, joint and muscle pain and inflammation, symptoms which are commonly shared among several autoimmune disorders. In addition, a patient can be affected by more than one autoimmune disease at the same time (such as concurrent Sjögren syndrome in SLE and RA patients) which confers an increased risk of misdiagnosis and/or under diagnosis (3, 4).

Current tools for clinical diagnosis include the combined information generated from clinical, laboratory and imaging findings, where the presence of various autoantibodies such as anti-nuclear antibodies (ANA), anti-cyclic citrullinated peptides (aCCP), Rheumatoid Factor (RF), anti-neutrophil cytoplasmic antibodies (ANCA), anti-double stranded antibodies (anti-dsDNA) and anti-Ro/SSA and anti-LA/SSB)), constitute important key players in the diagnostic routine of SLE, RA, SS and SV(5-8). However, a positive result for an autoantibody may not be exclusive for one disease and the use of single markers has not reached the high levels of specificity as required (9-12). Identification of new blood-based biomarkers for correct and early diagnosis is of high clinical relevance to enable early therapeutic interventions, thereby saving both lives and cost for society.

Considering that underlying disease biology is still unclear, panels of disease-specific markers can provide an improved option for reflecting underlying disease-specific molecular alterations. Previous studies have shown that high-performing proteomic technologies, such as recombinant antibody microarrays, offering a multiplexed approach are better able to reflect the complexity of multifactorial diseases, such as AID (13-18). Using this approach, candidate biomarker panels indicative for SLE, Systemic Sclerosis and SLE disease activity have been identified (11, 15).

However, there remains a need for improved methods of diagnosing or detecting autoimmune diseases, particularly SLE, RA, SS and SV.

### SUMMARY OF THE INVENTION

The inventors have now shown for the first time that by using biomarker panels classification of autoimmune disease could be achieved with high accuracy. These results highlight the power of using a multiplexed approached for decoding multifactorial, complex diseases such as autoimmune disease, which will play a significant role for diagnostic purposes.

Accordingly, a first aspect of the invention provides a method for diagnosing or detecting an autoimmune disease in an individual, the method comprising or consisting of the steps of:
a) providing a sample obtained from an individual to be tested; and
b) measuring the presence and/or amount in the test sample of one or more biomarkers selected from the group defined in Table 1(A);
   wherein the presence and/or amount in the sample of the one or more biomarker(s) selected from the group defined in Table 1(A) is indicative of an autoimmune disease in the individual.

TABLE 1(A)
Biomarkers for autoimmune disease

| Biomarker | UNIPROT ID |
|---|---|
| C3 | P01024 |
| IL-4 | P05112 |
| C5 | P01031 |
| C1 inh | P05155 |
| C4 | P0C0L4/5 |
| UBC9 | P63279 |
| IL-1ra | P18510 |
| CIMS | Antibodies selected against the |
| | peptide motifs TEEQLK/ |
| | SEAHLR/GIVKYLYEDEG |
| GM-CSF | P04141 |
| IL-1a | P01583 |
| TNF-a | P01375 |
| HADH2 | Q99714 |
| MAGI1-1 | Q96QZ7 |
| KCC2B-3 | Q13554-3 |
| LUM | P51884 |
| UPF3B | Q9BZI7 |
| GLP-1 R | P43220 |
| FASN | Q6PJJ3 |
| TopBP1 | Q92547 |
| IL-8 | P10145 |
| Factor B | P00751 |
| FER | P16591 |
| CDK-2 | P24941 |
| RPS6KA2 | Q15349 |
| GRK5 | P34947 |
| MAPKK 6 | P52564 |
| UBP7 | Q93009 |
| AGAP-2 | Q99490 |
| VEGF | P15692 |
| HLA-DR/DP | P01903/P01911/P79483/P13762/ |
| | Q30154/P20036/P04440 |
| GSN | P06396 |

Thus, in one embodiment, the method comprises determining a biomarker signature of the test sample, which enables a diagnosis to be reached in respect of the individual from which the sample is obtained.

By “autoimmune disease” we include any condition comprising or consisting of an abnormal immune response in an individual, wherein the immune response is directed against the individual.

By “diagnosing or detecting an autoimmune disease” we include determination of an autoimmune disease-associated state in an individual.

By “autoimmune disease-associated state” we include autoimmune disease diagnosis per se, the risk of having or of developing an autoimmune disease, and determination of the stage or sub-group of a particular autoimmune disease.

The term “autoimmune disease state” may mean or include (i) the presence or absence of an autoimmune disease (e.g., discriminating an active autoimmune disease from a non-autoimmune disease, a non-active autoimmune disease from a non-autoimmune disease and/or a highly active autoimmune disease from a non-autoimmune disease), and (ii) the activity of autoimmune disease (e.g., discriminating an active autoimmune disease from a non-active autoimmune disease, and/or discriminating a highly-active autoimmune disease from a non-active autoimmune disease).

Thus, it will be appreciated by persons skilled in the art that the methods of the invention are suitable for differentiating individuals with an autoimmune disease from healthy individuals as well as, for example, determining the activity level of an autoimmune disease in an individual (e.g. determining whether an autoimmune disease is in an active or inactive state) or determining whether an autoimmune disease is in remission in an individual.

Thus, in one embodiment, the method is for diagnosing an active autoimmune disease (e.g., an SLE flare) in a subject.

By “biomarker” we include any naturally-occurring biological molecule, or component or fragment thereof, the measurement of which can provide information useful in the diagnosis of an autoimmune disease. Thus, in the context of Table 1 generally (i.e. Table 1(A), Table 1(B), Table 1(C), Table 1(D) and Table 1(E)), the biomarker may be the protein, or a polypeptide fragment or carbohydrate moiety thereof (or, in the case of sialyl Lewis x, a carbohydrate moiety per se). Alternatively, the biomarker may be a nucleic acid molecule, such as a mRNA, cDNA or circulating tumour DNA molecule, which encodes the protein or part thereof.

In one embodiment, the biomarker mRNA and/or amino acid sequences correspond to those available on the GenBank database (http://www.ncbi.nlm.nih.gov/genbank/) and natural variants thereof. In a further embodiment, the biomarker mRNA and/or amino acid sequences correspond to those available on the GenBank database in January 2019.

In one embodiment of the invention, step (b) comprises or consists of measuring the presence and/or amount in the test sample of one or more of the biomarkers defined in Table 1(A), for example 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 of the biomarkers defined in Table 1(A).

For example, step (b) may comprise or consist of measuring at least 5 biomarkers. Step (b) may comprise or consist of measuring at least 10 biomarkers. Step (b) may comprise or consist of measuring at least 15 biomarkers. Step (b) may comprise or consist of measuring at least 20 biomarkers. Step (b) may comprise or consist of measuring 30 or fewer biomarkers. Step (b) may comprise or consist of measuring 25 or fewer biomarkers. Step (b) may comprise or consist of measuring 20-25 biomarkers. Step (b) may comprise or consist of measuring 25-31 biomarkers.

In an additional or alternative embodiment of any of the aspects of the invention described herein, in step (b) the presence and/or amount in the test sample of GSN (gelsolin) is measured in addition to the presence and/or amount of HADH2.

In an additional or alternative embodiment of any of the aspects of the invention described herein, in step (b) the presence and/or amount in the test sample of GSN (gelsolin) is measured instead of the presence and/or amount of HADH2. In an additional or alternative embodiment of each of the aspects of the invention described herein, in step (b) the presence and/or amount in the test sample of HADH2 is measured instead of GSN (gelsolin).

As detailed in Supplementary Table S6, the antibody sequence referred to herein as binding HADH2 may also bind GSN.

In an additional or alternative embodiment of any of the aspects of the invention described herein, measuring the presence and/or amount in the test sample of HADH2 and/or GSN in step (b) is replaced by measuring the presence and/or amount in the test sample of a protein bound by the antibody sequence of SEQ ID NO: 7. Preferably the protein bound by the antibody sequence of SEQ ID NO: 7 is HADH2 and/or GSN.

In an additional or alternative embodiment of any of the aspects of the invention described herein, measuring the presence and/or amount in the test sample of one or more core biomarkers in step (b) is replaced by measuring the presence and/or amount in the test sample of a protein bound by one or more of the antibody sequences described in Supplementary Table S6.

It will be appreciated that step (b) may additionally comprise measuring the presence and/or amount of one or more further biomarkers not listed in Table 1(A), wherein the further biomarkers may provide additional diagnostic information.

In one embodiment step (b) comprises or consists of measuring the presence and/or amount in the test sample of one or more of the biomarkers defined in Table 1(A)i, i.e. step (b) comprises on consists of measuring the presence and/or amount of one or more “core biomarker”, for example 2 or 3 of the core biomarkers.

TABLE 1(A)i
core biomarkers for the diagnosis
of autoimmune disease

| Biomarker | UNIPROT ID |
|---|---|
| HADH2 | Q99714 |
| FER | P16591 |
| GSN | P06396 |

In one embodiment step (b) comprises or consists of measuring the presence and/or amount in the test sample of one or more, for example 2, 3, 4, 5, 6, 7, 8, 9, 10, 11, 12, 13, 14, 15, 16, 17, 18, 19, 20, 21, or 22 of the biomarkers defined in Table 1(A)ii, i.e. step (b) comprises on consists of measuring the presence and/or amount of one or more “preferred biomarker”.

TABLE 1(A)ii
preferred biomarkers for the
diagnosis of autoimmune disease

| Biomarker | UNIPROT ID |
|---|---|
| UBC9 | P63279 |
| C3 | P01024 |
| C4 | P0C0L4/5 |
| TNF-a | P01375 |
| IL-1ra | P18510 |
| C1 esterase inhibitor | P05155 |
| IL-4 | P05112 |
| GM-CSF | P04141 |
| IL-1a | P01583 |
| MAGI1-1 | Q96QZ7 |
| LUM | P51884 |
| GLP-1 R | P43220 |
| IL-8 | P10145 |
| TopBP1 | Q92547 |
| Factor B | P00751 |
| CDK-2 | P24941 |
| GRK5 | P34947 |
| MAPKK 6 | P52564 |
| VEGF | P15692 |
| HLA-DR/DP | P01903/P01911/P79483/ |
| | P13762/Q30154/P20036/ |
| | P04440 |
| KCC2B-3 | Q13554-3 |
| C5 | P01031 |

In one embodiment step (b) comprises or consists of measuring the presence and/or amount in the test sample of one or more, for example 2, 3, 4, 5, or 6, of the biomarkers defined in Table 1(A)iii, i.e. step (b) comprises on consists of measuring the presence and/or amount of one or more “optional biomarker”.

TABLE 1(A)iii
optional biomarkers for the
diagnosis of autoimmune disease

| Biomarker | UNIPROT ID |
|---|---|
| CIMS | Antibodies selected |
| | against the peptide |
| | motifs TEEQLK/ |
| | SEAHLR/ |
| | GIVKYLYEDEG |
| UPF3B | Q9BZI7 |
| FASN | Q6PJJ3 |
| RPS6KA2 | Q15349 |
| UBP7 | Q93009 |
| AGAP-2 | Q99490 |

In one embodiment step (b) comprises or consists of measuring the presence and/or amount in the test sample of biomarkers defined in Table 1(A)i, Table 1(A)ii and/or Table 1(A)iii. i.e. step (b) comprises measuring the presence of core, preferred and/or biomarkers.

In one embodiment the one or more biomarker(s) selected from the group defined in Table 1(A) are biomarkers which are also present in Table 2(A). Table 2(A) corresponds to differentially expressed markers in autoimmune disease.

TABLE 2(A)

| Biomarker | UNIPROT ID |
|---|---|
| C3 | P01024 |
| UBC9 | P63279 |
| RPS6KA2 | Q15349 |
| CIMS | Antibodies |
| | selected against |
| | peptide motif |
| | TEEQLK |
| STAP1 | Q9ULZ2 |
| P85A | P27986 |
| KCC2B-3 | Q13554-3 |
| INADL-1 | Q8NI35 |
| DLG4-2 | P78352-2 |
| Osteopontin | P10451 |
| UCHL5 | Q9Y5K5 |
| C4 | P0C0L4/5 |
| MARK1-1 | Q9P0L2-1 |
| C5 | P01031 |
| PRKCZ | Q05513 |
| KKCC1-1 | Q8N5S9-1 |
| PTPN1 | P18031 |
| hSpindly | Q96EA4 |

In one embodiment of the first aspect of the invention, the method further comprises measuring the presence and/or amount of one or more of the biomarkers defined in Table 2(A). It will be appreciated by persons skilled in the art that these markers may be different to those in Table 1(A). Thus, the method may comprise a further additional step of measuring markers present in Table 2(A) (differentially expressed markers) which are not present in Table 1(A).

It will be appreciated by persons skilled in the art that the biomarker signature of Table 1(A), directed to autoimmune diseases generally, may be used in combination with any one or more of the biomarker signatures of Table 1(B), Table 1(C), Table 1(D), and Table 1(E), relating to specific autoimmune diseases (SLE, RA, SS and SV, respectively).

TABLE 1(B)
Biomarkers for systemic
lupus erythematosus

| Biomarker | UNIPROT ID |
|---|---|
| C3 | P01024 |
| C4 | P0C0L4/5 |
| MAGI1-1 | Q96QZ7 |
| VEGF | P15692 |
| Apo-A1 | P02647 |
| C1 inh. | P05155 |
| OTUB1-1 | Q96FW1-1 |
| C5 | P01031 |
| IL-6 | P05231 |
| IL-4 | P05112 |
| PAK7 | Q9P286 |
| TNF-a | P01375 |
| Lewis x | Carbohydrate moiety |
| GM-CSF | P04141 |
| NOS1-1 | P29475 |
| DPOLM | Q9NP87 |
| TNFRSF3 | P36941 |
| IL-1ra | P18510 |
| OTUB2-1 | Q96DC9-1 |
| IL-13 | P35225 |
| MCP-1 | P13500 |
| MATK | P42679 |
| MUC1 | P15941 |
| IgM | Immunoglobulin M |
| PTPPRN2 | Q92932 |
| OTU6B | Q8N6M0 |
| MARK2-1 | Q7KZI7-1 |
| BIRC2 | Q13490 |
| hSpindly | Q96EA4 |
| HADH2 | Q99714 |
| MAPKK 6 | P52564 |
| GSN | P06396 |

TABLE 1(C)
Biomarkers for rheumatoid arthritis

| Biomarker | UNIPROT ID |
|---|---|
| C3 | P01024 |
| VEGF | P15692 |
| KKCC1-1 | Q8N5S9-1 |
| CIMS | Antibodies selected against peptide |
| | motifs GIVKYLYEDEG/DFAEDK/ |
| | LTEFAK/TEEQLK/SSAYSR |
| Factor B | P00751 |
| PRKG2 | Q13237 |
| C1 inh. | P05155 |
| GM-CSF | P04141 |
| PKB gamma | Q9Y243 |
| C5 | P01031 |
| MCP-4 | Q99616 |
| LUM | P51884 |
| PTN13-1 | Q12923-1 |
| IgM | Immunoglobulin M |
| EGFR | P00533 |
| NOS1-1 | P29475 |
| Procathepsin W | P56202 |
| Apo-A4 | P06727 |
| IL-9 | P15248 |
| PTK6 | Q13882 |
| hSpindly | Q96EA4 |
| GRIP-2 | Q9C0E4 |
| CHEK2 | O96017 |
| MAPKK 2 | P36507 |
| ITCH-2 | Q96J02-2 |
| Cystatin C | P01034 |
| PRKCZ | Q05513 |
| IL-1ra | P18510 |
| IL-4 | P05112 |

TABLE 1(D)
Biomarkers for Sjögren's syndrome

| Biomarker | UNIPROT ID |
|---|---|
| C1 inh. | P05155 |
| OTUB1-1 | Q96FW1-1 |
| MCP-1 | P13500 |
| HADH2 | Q99714 |
| Angiomotin | Q4VCS5 |
| IL-13 | P35225 |
| PRKG2 | Q13237 |
| Factor B | P00751 |
| CIMS | Antibodies selected |
| | against peptide motifs |
| | DFAEDK/LTEFAK/ |
| | LSADHR/SEAHLR |
| CSNK1E | P49674 |
| Apo-A1 | P02647 |
| IL-6 | P05231 |
| IL-3 | P08700 |
| PTPRD | P23468 |
| ARHGC-1 | Q9NZN5 |
| Properdin | P27918 |
| DCNL1 | Q96GG9 |
| GAK | Q5U4P5 |
| TBC1D9 | Q6ZT07 |
| TNFRSF14 | Q92956 |
| MARK2-1 | Q7KZI7-1 |
| Sox11a | P35716 |
| KKCC1-1 | Q8N5S9-1 |
| MUC1 | P15941 |
| GEM | P55040 |
| MARK1-1 | Q9P0L2-1 |
| C5 | P01031 |
| RANTES | P13501 |
| IL-12 | P29459/60 |
| MAPKK 6 | P52564 |
| IL-8 | P10145 |
| UPF3B | Q9BZI7 |
| GSN | P06396 |

TABLE 1(E)
Biomarkers for systemic vasculitis

| Biomarker | UNIPROT ID |
|---|---|
| Factor B | P00751 |
| Apo-A1 | P02647 |
| C3 | P01024 |
| CIMS | Antibodies selected against |
| | peptide motifs TEEQLK/ |
| | SSAYSR/LSADHR/ |
| | SEAHLR |
| IgM | Immunoglobulin M |
| C4 | P0C0L4/5 |
| KKCC1-1 | Q8N5S9-1 |
| IL-4 | P05112 |
| C1 inh. | P05155 |
| C1q | P02745/6/7 |
| Angiomotin | Q4VCS5 |
| PTPRD | P23468 |
| MCP-1 | P13500 |
| IL-3 | P08700 |
| GM-CSF | P04141 |
| IL-12 | P29459/60 |
| C5 | P01031 |
| Cystatin C | P01034 |
| IL-18 | Q14116 |
| MUC1 | P15941 |
| DLG4-2 | P78352-2 |
| UPF3B | Q9BZI7 |
| CT17 | E3UUX3 |
| TNFRSF14 | Q92956 |
| MAPKK 6 | P52564 |
| TNFRSF3 | P36941 |

Thus, in one embodiment, the method further comprises measuring the presence and/or amount of one or more, for example 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 or 32, of the biomarkers defined in Table 1(B).

In one embodiment, the method further comprises measuring the presence and/or amount of one or more, for example 2, 3, 4, 5, 6, 7, 8, 9, 10, 11, 12, 13, 14, 15, 16, 17, 18, 19, 20, 21, 22, 23, 24, 25, 26, 27, 28, or 29, of the biomarkers defined in Table 1(C).

In one embodiment, the method further comprises measuring the presence and/or amount of one or more, for example 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or 33, of the biomarkers defined in Table 1(D).

In one embodiment, the method further comprises measuring the presence and/or amount of one or more, for example 2, 3, 4, 5, 6, 7, 8, 9, 10, 11, 12, 13, 14, 15, 16, 17, 18, 19, 20, 21, 22, 23, 24, 25, or 26, the biomarkers defined in Table 1(E).

In one embodiment of the invention, the automimmune disease to be diagnosed is an inflammatory rheumatic disease, e.g. systemic lupus erythematosus (SLE), rheumatoid arthritis (RA), Sjögren's syndrome (SS) or systemic vasculitis (SV).

In one embodiment of the invention, the autoimmune disease to be diagnosed is selected from: systemic lupus erythematosus (SLE), rheumatoid arthritis (RA), Sjögren's syndrome (SS) or systemic vasculitis (SV).

In one embodiment of the invention, systemic vasculitis (SV) is antineutrophil cytoplasmic antibody (ANCA) associated vasculitis.

Also provided as part of the invention are specific methods for diagnosing or detecting specific autoimmune diseases. It will be appreciated by persons skilled in the art that the descriptions and options relating to the first aspect of the invention also apply for these subsequent aspects of the present invention, as they are closely related methods.

Therefore a second, related, aspect of the invention provides a method for diagnosing or detecting systemic lupus erythematosus in an individual comprising or consisting of the steps of:
a) providing one or more sample obtained from an individual with, or suspected of having, an autoimmune disease; and
b) measuring the presence and/or amount in the test sample of one or more biomarker selected from the group defined in Table 1(B);
   wherein the presence and/or amount in the one or more test sample of the one or more biomarker(s) selected from the group defined in Table 1(B) is indicative of systemic lupus erythematosus.

In one embodiment of the invention, step (b) comprises or consists of measuring the presence and/or amount in the test sample of one or more of the biomarkers defined in Table 1(B), for example 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 32 of the biomarkers defined in Table 1(B).

For example, step (b) may comprise or consist of measuring at least 5 biomarkers. Step (b) may comprise or consist of measuring at least 10 biomarkers. Step (b) may comprise or consist of measuring at least 15 biomarkers. Step (b) may comprise or consist of measuring at least 20 biomarkers. Step (b) may comprise or consist of measuring 32 or fewer biomarkers. Step (b) may comprise or consist of measuring 25 or fewer biomarkers. Step (b) may comprise or consist of measuring 20-25 biomarkers. Step (b) may comprise or consist of measuring 25-32 biomarkers.

It will be appreciated that step (b) may additionally comprise measuring the presence and/or amount of one or more further biomarkers not listed in Table 1(B), wherein the further biomarkers may provide additional diagnostic information.

In one embodiment step (b) comprises or consists of measuring the presence and/or amount in the test sample of one or more, for example 2, or 3, of the biomarkers defined in Table 1(B)i, i.e. step (b) comprises on consists of measuring the presence and/or amount of one or more “core biomarker”.

TABLE 1(B)i
core biomarkers for the diagnosis of
systemic lupus erythematosus (SLE).

| Biomarker | UNIPROT ID |
|---|---|
| MAGI1-1 | Q96QZ7 |
| OTUB1-1 | Q96FW1-1 |
| PAK7 | Q9P286 |

In one embodiment step (b) comprises or consists of measuring the presence and/or amount in the test sample of one or more, for example 2, 3, 4, 5, 6, 7, 8, 9, 10, 11, 12, 13, 14, 15, 16, 17, 18, or 19 of the biomarkers defined in Table 1(B)ii, i.e. step (b) comprises on consists of measuring the presence and/or amount of one or more “preferred biomarker”.

TABLE 1(B)ii
preferred biomarkers for the diagnosis
of systemic lupus erythematosus (SLE).

| Biomarker | UNIPROT ID |
|---|---|
| C3 | P01024 |
| C4 | P0C0L4/5 |
| VEGF | P15692 |
| Apo-A1 | P02647 |
| IL-6 | P05231 |
| IL-4 | P05112 |
| TNF-a | P01375 |
| GM-CSF | P04141 |
| Lewis x | Carbohydrate moiety |
| NOS1-1 | P29475 |
| TNFRSF3 | P36941 |
| IL-1ra | P18510 |
| IL-13 | P35225 |
| MCP-1 | P13500 |
| OTU6B | Q8N6M0 |
| BIRC2 | Q13490 |
| MAPKK 6 | P52564 |
| C1 inh. (1) | P05155 |
| C5 (3) | P01031 |

In one embodiment step (b) comprises or consists of measuring the presence and/or amount in the test sample of one or more, for example 2, 3, 4, 5, 6, 7, 8, 9 or 10, of the biomarkers defined in Table 1(B)iii, i.e. step (b) comprises on consists of measuring the presence and/or amount of one or more “optional biomarker”.

TABLE 1(B)iii
optional biomarkers for the diagnosis
of systemic lupus erythematosus (SLE).

| Biomarker | UNIPROT ID |
|---|---|
| DPOLM | Q9NP87 |
| OTUB2 | Q96DC9-1 |
| MATK | P42679 |
| MUC1 | P15941 |
| IgM | Immunoglobulin M |
| PTPPRN2 | Q92932 |
| MARK2-1 | Q7KZI7-1 |
| hSpindly | Q96EA4 |
| HADH2 | Q99714 |
| GSN | P06396 |

In one embodiment step (b) comprises or consists of measuring the presence and/or amount in the test sample of biomarkers defined in Table 1(B)i, Table 1(B)ii and/or Table 1(B)iii. i.e. step (b) comprises measuring the presence of core, preferred and/or biomarkers.

In one embodiment the one or more biomarker(s) selected from the group defined in Table 1(B) are biomarkers which are also present in Table 2(B). Table 2(B) corresponds to differentially expressed markers in SLE.

TABLE 2(B)
differentially expressed markers for SLE

| Biomarker | UNIPROT ID |
|---|---|
| C4 | P0C0L4/5 |
| C3 | P01024 |
| Apo-A1 | P02647 |
| VEGF | P15692 |
| MAGI1-1 | Q96QZ7 |
| C1 inh. | P05155 |
| IgM | Immunoglobulin M |
| NOS1-1 | P29475 |
| BIRC2 | Q13490 |
| GM-CSF | P04141 |
| GAK | Q5U4P5 |
| TNFRSF14 | Q92956 |
| Lewis y | carbohydrate moiety |
| IL-4 | P05112 |
| MCP-1 | P13500 |
| Cystatin C | P01034 |

In one embodiment the method further comprises measuring the presence and/or amount of one or more of the biomarkers defined in Table 2(B). It will be appreciated by persons skilled in the art that the markers to be measured may or may not also be present in Table 1(B).

A third aspect of the invention provides a method for diagnosing or detecting rheumatoid arthritis (RA) in an individual comprising or consisting of the steps of:
a) providing one or more sample obtained from an individual with, or suspected of having, an autoimmune disease; and
b) measuring the presence and/or amount in the test sample of one or more biomarker selected from the group defined in Table 1(C);
   wherein the presence and/or amount in the one or more test sample of the one or more biomarker(s) selected from the group defined in Table 1(C) is indicative of rheumatoid arthritis (RA).

In one embodiment of the invention, step (b) comprises or consists of measuring the presence and/or amount in the test sample of one or more of the biomarkers defined in Table 1(C), for example 1, 2, 3, 4, 5, 6, 7, 8, 9, 10, 11, 12, 13, 14, 15, 16, 17, 18, 19, 20, 21, 22, 23, 24, 25, 26, 27, 28 or 29 of the biomarkers defined in Table 1(C).

For example, step (b) may comprise or consist of measuring at least 5 biomarkers. Step (b) may comprise or consist of measuring at least 10 biomarkers. Step (b) may comprise or consist of measuring at least 15 biomarkers. Step (b) may comprise or consist of measuring at least 20 biomarkers. Step (b) may comprise or consist of measuring 29 or fewer biomarkers. Step (b) may comprise or consist of measuring 25 or fewer biomarkers. Step (b) may comprise or consist of measuring 20-25 biomarkers. Step (b) may comprise or consist of measuring 25-29 biomarkers.

It will be appreciated that step (b) may additionally comprise measuring the presence and/or amount of one or more further biomarkers not listed in Table 1(C), wherein the further biomarkers may provide additional diagnostic information.

In one embodiment step (b) comprises or consists of measuring the presence and/or amount in the test sample of one or more, for example 2, or 3, of the biomarkers defined in Table 1(C)i, i.e. step (b) comprises on consists of measuring the presence and/or amount of one or more “core biomarker”.

TABLE 1(C)i
core biomarkers for the diagnosis of RA

| Biomarker | UNIPROT ID |
|---|---|
| KKCC1-1 | Q8N5S9-1 |
| Factor B | P00751 |
| Procathepsin W | P56202 |

In one embodiment step (b) comprises or consists of measuring the presence and/or amount in the test sample of one or more, for example 2, 3, 4, 5, 6, 7, 8, 9, 10, 11, 12, 13, or 14, of the biomarkers defined in Table 1(C)ii, i.e. step (b) comprises on consists of measuring the presence and/or amount of one or more “preferred biomarker”.

TABLE 1(C)ii
preferred biomarkers for the diagnosis of RA

| Biomarker | UNIPROT ID |
|---|---|
| C3 | P01024 |
| VEGF | P15692 |
| GM-CSF | P04141 |
| C5 | P01031 |
| MCP-4 | Q99616 |
| EGFR | P00533 |
| NOS1-1 | P29475 |
| Apo-A4 | P06727 |
| IL-9 | P15248 |
| CHEK2 | 096017 |
| Cystatin C | P01034 |
| PRKCZ | Q05513 |
| IL-1ra | P18510 |
| IL-4 | P05112 |

In one embodiment step (b) comprises or consists of measuring the presence and/or amount in the test sample of one or more, for example 2, 3, 4, 5, 6, 7, 8, 9, 10, 11, or 12 of the biomarkers defined in Table 1(C)iii, i.e. step (b) comprises on consists of measuring the presence and/or amount of one or more “optional biomarker”.

TABLE 1(C)iii
optional biomarkers for the diagnosis of RA

| Biomarker | UNIPROT ID |
|---|---|
| CIMS | Antibodies selected against peptide |
| | motifs GIVKYLYEDEG/ DFAEDK/ |
| | LTEFAK/ TEEQLK/ SSAYSR |
| LUM | P51884 |
| PTN13-1 | Q12923-1 |
| PTK6 | Q13882 |
| hSpindly | Q96EA4 |
| GRIP-2 | Q9C0E4 |
| MAPKK 2 | P36507 |
| ITCH-2 | Q96J02-2 |
| IgM | Immunoglobulin M |
| PRKG2 | Q13237 |
| C1 inh. | P05155 |
| PKB gamma | Q9Y243 |

In one embodiment step (b) comprises or consists of measuring the presence and/or amount in the test sample of biomarkers defined in Table 1(C)i, Table 1(C)ii and/or Table 1(C)iii. i.e. step (b) comprises measuring the presence of core, preferred and/or biomarkers.

In one embodiment the one or more biomarker(s) selected from the group defined in Table 1(C) are biomarkers which are also present in Table 2(C). Table 2(C) corresponds to differentially expressed markers in RA.

TABLE 2(C)
differentially expressed markers in RA

| Biomarker | UNIPROT ID |
|---|---|
| C1 inh. | P05155 |
| PRKG2 | Q13237 |
| KKCC1-1 | Q8N5S9-1 |
| PTN13-1 | Q12923-1 |
| IgM | Immunoglobulin M |
| PKB gamma | Q9Y243 |
| CDK-2 | P24941 |
| MAPKK 2 | P36507 |
| GLP-1 | P01275 |
| OTUB2-1 | Q96DC9-1 |
| C5 | P01031 |
| IL-6 | P05231 |
| SNTA1 | Q13424 |
| CHEK2 | 096017 |
| MATK | P42679 |
| IL-1b | P01584 |
| TNFRSF3 | P36941 |
| DLG4-2 | P78352-2 |
| IL-8 | P10145 |
| TNF-b | P01374 |
| Lewis y | Carbohydrate antigen |
| NOS1-1 | P29475 |

In one embodiment of the first aspect of the invention, the method further comprises measuring the presence and/or amount of one or more of the biomarkers defined in Table 2(C). It will be appreciated by persons skilled in the art that these markers may be different to those in Table 1(C). Thus, the method may comprise a further additional step of measuring markers present in Table 2(C) (differentially expressed markers) which are not present in Table 1(C).

In one embodiment the one or more biomarker(s) selected from the group defined in Table 1(C) are biomarkers which are also present in Table 2(C).

In one embodiment the method further comprises measuring the presence and/or amount of one or more of the biomarkers defined in Table 2(C). It will be appreciated by persons skilled in the art that the markers to be measured may or may not also be present in Table 1(C).

A fourth aspect of the invention provides a method for diagnosing or detecting Sjögren's syndrome (SS) in an individual comprising or consisting of the steps of:
a) providing one or more sample obtained from an individual with, or suspected of having, an autoimmune disease; and
b) measuring the presence and/or amount in the test sample of one or more biomarker selected from the group defined in Table 1(D);
   wherein the presence and/or amount in the one or more test sample of the one or more biomarker(s) selected from the group defined in Table 1(D) is indicative of Sjögren's syndrome (SS).

In one embodiment of the invention, step (b) comprises or consists of measuring the presence and/or amount in the test sample of one or more of the biomarkers defined in Table 1(D), for example 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 32 or 33 of the biomarkers defined in Table 1(D).

For example, step (b) may comprise or consist of measuring at least 5 biomarkers. Step (b) may comprise or consist of measuring at least 10 biomarkers. Step (b) may comprise or consist of measuring at least 15 biomarkers. Step (b) may comprise or consist of measuring at least 20 biomarkers. Step (b) may comprise or consist of measuring 30 or fewer biomarkers. Step (b) may comprise or consist of measuring 25 or fewer biomarkers. Step (b) may comprise or consist of measuring 20-25 biomarkers. Step (b) may comprise or consist of measuring 25-30 biomarkers. Step (b) may comprise or consist of measuring 30-33 biomarkers.

It will be appreciated that step (b) may additionally comprise measuring the presence and/or amount of one or more further biomarkers not listed in Table 1(D), wherein the further biomarkers may provide additional diagnostic information.

In one embodiment step (b) comprises or consists of measuring the presence and/or amount in the test sample of one or more, for example 2, or 3, of the biomarkers defined in Table 1(D)i, i.e. step (b) comprises on consists of measuring the presence and/or amount of one or more “core biomarker”.

TABLE 1(D)i
core biomarkers for the diagnosis of SS.

| Biomarker | UNIPROT ID |
|---|---|
| OTUB1-1 | Q96FW1-1 |
| MCP-1 | P13500 |
| Factor B | P00751 |

In one embodiment step (b) comprises or consists of measuring the presence and/or amount in the test sample of one or more, for example 2, 3, 4, 5, 6, 7, 8, or 9, of the biomarkers defined in Table 1(D)ii, i.e. step (b) comprises on consists of measuring the presence and/or amount of one or more “preferred biomarker”.

TABLE 1(D)ii
preferred biomarkers for the diagnosis of SS.

| Biomarker | UNIPROT ID |
|---|---|
| C1 inh. | P05155 |
| IL-13 | P35225 |
| IL-6 | P05231 |
| MARK2-1 | Q7KZI7-1 |
| MUC1 | P15941 |
| C5 | P01031 |
| RANTES | P13501 |
| IL-12 | P29459/60 |
| IL-8 | P10145 |

In one embodiment step (b) comprises or consists of measuring the presence and/or amount in the test sample of one or more, for example 2, 3, 4, 5, 6, 7, 8, 9, 10, 11, 12, 13, 14, 15, 16, 17, 18, 19, or 20 21, of the biomarkers defined in Table 1(D)iii, i.e. step (b) comprises on consists of measuring the presence and/or amount of one or more “optional biomarker”.

TABLE 1(D)iii
optional biomarkers for the diagnosis of SS.

| Biomarker | UNIPROT ID |
|---|---|
| HADH2 | Q99714 |
| Angiomotin | Q4VCS5 |
| PRKG2 | Q13237 |
| CSNK1E | P49674 |
| Apo-A1 | P02647 |
| PTPRD | P23468 |
| ARHGC-1 | Q9NZN5 |
| Properdin | P27918 |
| DCNL1 | Q96GG9 |
| GAK | Q5U4P5 |
| TBC1D9 | Q6ZT07 |
| TNFRSF14 | Q92956 |
| Sox11a | P35716 |
| KKCC1-1 | Q8N5S9-1 |
| GEM | P55040 |
| MARK1-1 | Q9P0L2-1 |
| MAPKK 6 | P52564 |
| UPF3B | Q9BZI7 |
| CIMS | Antibodies selected against |
| | peptide motifs DFAEDK/ |
| | LTEFAK/ LSADHR/ SEAHLR |
| IL-3 | P08700 |
| GSN | P06396 |

In one embodiment step (b) comprises or consists of measuring the presence and/or amount in the test sample of biomarkers defined in Table 1(D)i, Table 1(D)ii and/or Table 1(D)iii. i.e. step (b) comprises measuring the presence of core, preferred and/or biomarkers.

In one embodiment the one or more biomarker(s) selected from the group defined in Table 1(D) are biomarkers which are also present in Table 2(D). Table 2(D) corresponds to differentially expressed markers in SS.

TABLE 2(D)
differentially expressed biomarkers in SS.

| Biomarker | UNIPROT ID |
|---|---|
| MATK | P42679 |
| GEM | P55040 |
| Her2/ErbB2 | P04626 |
| PAR-6B | Q9BYG5 |
| DCNL1 | Q96GG9 |
| ITCH-2 | Q96J02-2 |
| FER | P16591 |
| KSYK | P43405 |
| HADH2 | Q99714 |
| CSNK1E | P49674 |
| GORS2-1 | Q9H8Y8 |
| IL-3 | P08700 |
| TNFRSF14 | Q92956 |
| UBP7 | Q93009 |
| R-PTP-eta | Q12913 |
| LIN7A | 014910 |
| RANTES | P13501 |
| KCC2B-3 | Q13554-3 |
| CHP1 | Q99653 |
| IL-1b | P01584 |
| GSN | P06396 |

In one embodiment the method further comprises measuring the presence and/or amount of one or more of the biomarkers defined in Table 2(D). It will be appreciated by persons skilled in the art that the markers to be measured may or may not also be present in Table 1(D).

A fifth aspect of the invention provides a method for diagnosing or detecting systemic vasculitis (SV) in an individual comprising or consisting of the steps of:
a) providing one or more sample obtained from an individual with, or suspected of having, an autoimmune disease; and
b) measuring the presence and/or amount in the test sample of one or more biomarker selected from the group defined in Table 1(E);
   wherein the presence and/or amount in the one or more test sample of the one or more biomarker(s) selected from the group defined in Table 1(E) is indicative of systemic vasculitis (SV).

In one embodiment of the invention, the systemic vasculitis (SV) is antineutrophil cytoplasmic antibody (ANCA) associated vasculitis.

In one embodiment of the invention, step (b) comprises or consists of measuring the presence and/or amount in the test sample of one or more of the biomarkers defined in Table 1(E), for example 1, 2, 3, 4, 5, 6, 7, 8, 9, 10, 11, 12, 13, 14, 15, 16, 17, 18, 19, 20, 21, 22, 23, 24, 25, 26 or 27 of the biomarkers defined in Table 1(E).

For example, step (b) may comprise or consist of measuring at least 5 biomarkers. Step (b) may comprise or consist of measuring at least 10 biomarkers. Step (b) may comprise or consist of measuring at least 15 biomarkers. Step (b) may comprise or consist of measuring at least 20 biomarkers. Step (b) may comprise or consist of measuring 27 or fewer biomarkers. Step (b) may comprise or consist of measuring 25 or fewer biomarkers. Step (b) may comprise or consist of measuring 20-25 biomarkers. Step (b) may comprise or consist of measuring 25-27 biomarkers.

It will be appreciated that step (b) may additionally comprise measuring the presence and/or amount of one or more further biomarkers not listed in Table 1(E), wherein the further biomarkers may provide additional diagnostic information.

In one embodiment step (b) comprises or consists of measuring the presence and/or amount in the test sample of one or more, for example 2, or 3, of the biomarkers defined in Table 1(E)i, i.e. step (b) comprises on consists of measuring the presence and/or amount of one or more “core biomarker”.

TABLE 1(E)i
core biomarkers for the diagnosis of SV.

| Biomarker | UNIPROT ID |
|---|---|
| KKCC1-1 | Q8N5S9-1 |
| IL-4 | P05112 |
| UPF3B | Q9BZI7 |

In one embodiment step (b) comprises or consists of measuring the presence and/or amount in the test sample of one or more, for example 2, 3, 4, 5, 6, 7, 8, 9, 10, 11, 12, or 13 of the biomarkers defined in Table 1(E)ii, i.e. step (b) comprises on consists of measuring the presence and/or amount of one or more “preferred biomarker”.

TABLE 1(E)ii
preferred biomarkers for the diagnosis of SV.

| Biomarker | UNIPROT ID |
|---|---|
| Factor B | P00751 |
| Apo-A1 | P02647 |
| C3 | P01024 |
| C4 | P0C0L4/5 |
| C1 inh. | P05155 |
| MCP-1 | P13500 |
| IL-3 | P08700 |
| GM-CSF | P04141 |
| IL-12 | P29459/60 |
| C5 | P01031 |
| Cystatin C | P01034 |
| IL-18 | Q14116 |
| MUC1 | P15941 |

In one embodiment step (b) comprises or consists of measuring the presence and/or amount in the test sample of one or more, for example 2, 3, 4, 5, 6, 7, 8, 9, or 10, of the biomarkers defined in Table 1(E)iii, i.e. step (b) comprises on consists of measuring the presence and/or amount of one or more “optional biomarker”.

TABLE 1(E)iii
optional biomarkers for the diagnosis of SV.

| Biomarker | UNIPROT ID |
|---|---|
| CIMS | Antibodies selected against peptide motifs |
| | TEEQLK/ SSAYSR/ LSADHR/ SEAHLR |
| IgM | Immunoglobulin M |
| C1q | P02745/6/7 |
| Angiomotin | Q4VCS5 |
| PTPRD | P23468 |
| DLG4-2 | P78352-2 |
| CT17 | E3UUX3 |
| TNFRSF14 | Q92956 |
| MAPKK 6 | P52564 |
| TNFRSF3 | P36941 |

In one embodiment step (b) comprises or consists of measuring the presence and/or amount in the test sample of biomarkers defined in Table 1(E)i, Table 1(E)ii and/or Table 1(E)iii. i.e. step (b) comprises measuring the presence of core, preferred and/or biomarkers.

In one embodiment the one or more biomarker(s) selected from the group defined in Table 1(E) are biomarkers which are also present in Table 2(E). Table 2(E) corresponds to differentially expressed markers in SV.

TABLE 2(E)
differentially expressed biomarkers in SV.

| Biomarker | UNIPROT ID |
|---|---|
| IgM | Immunoglobulin M |
| IL-4 | P05112 |
| Angiomotin | Q4VCS5 |
| GM-CSF | P04141 |
| MUC1 | P15941 |
| IL-11 | P20809 |
| PSA | P07288 |
| RANTES | P13501 |
| IL-12 | P29459/60 |
| UPF3B | Q9BZI7 |
| IL-3 | P08700 |
| CT17 | E3UUX3 |
| PTPRD | P23468 |
| BTK | Q06187 |
| TNF-b | P01374 |
| IL-10 | P22301 |
| MCP-4 | Q99616 |
| CD40 ligand | P29965 |
| TBC1D9 | Q6ZT07 |
| PAR-6B | Q9BYG5 |
| C4 | P0C0L4/5 |

In one embodiment the one or more biomarker(s) selected from the group defined in Table 1(E) are biomarkers which are also present in Table 2(E).

In one embodiment the method further comprises measuring the presence and/or amount of one or more of the biomarkers defined in Table 2(E). It will be appreciated by persons skilled in the art that the markers to be measured may or may not also be present in Table 1(E).

It will be appreciated by persons skilled in the art that specified embodiments may be applied to any of the first to fifth aspects of the invention, as the first to the fifth aspects of the invention all relate to closely related methods.

For example, in any of the first to the fifth aspects of the invention, preferably the individual is a human, but may be any mammal such as a domesticated mammal (preferably of agricultural or commercial significance including a horse, pig, cow, sheep, dog and cat).

For the avoidance of doubt, test samples from more than one disease state may be provided in step (a), for example, ≥2, ≥3, ≥4, ≥5, ≥6 or ≥7 or different disease states. Step (a) may provide at least two test samples, for example, ≥3, ≥4, ≥5, ≥6, ≥7, ≥8, ≥9, ≥10, ≥15, ≥20, ≥25, ≥50 or ≥100 test samples. Where multiple test samples are provided, they may be of the same type (e.g., all serum or urine samples) or of different types (e.g., serum and urine samples).

It will be appreciated by persons skilled in the art that, in addition to measuring the biomarkers in a sample from an individual to be tested, the methods of the invention may also comprise measuring those same biomarkers in one or more control samples.

Thus, in one embodiment, the method of any of the above aspects of the invention further comprises the steps of:
c) providing one or more control samples; and
d) measuring the presence and/or amount in the control sample of the one or more biomarkers measured in step (b);
   wherein the individual is identified as having an autoimmune disease by comparing the presence and/or amount in the test sample of the one or more biomarkers measured in step (b) with the presence and/or amount in the control samples measured in step (d).

As discussed above, by “having an autoimmune disease” we include both diagnosis of an autoimmune disease and determination of an autoimmune disease-associated state.

Optionally the control samples of step (c) are provided from an individual not having an autoimmune disease (negative control). Optionally, the individual not afflicted with an autoimmune disease is a healthy individual (negative control).

Alternatively, or additionally, the control samples of step (c) are provided from an individual with an autoimmune disease (positive control).

Thus, the individual may be identified as having an autoimmune disease in the event that the presence and/or amount in the test sample of the one or more biomarkers measured in step (b) is different from the presence and/or amount in the control sample. Alternatively, the presence and/or amount in the test sample of the one or more biomarkers measured in step (b) corresponds to the presence and/or amount in the control sample of the one or more biomarkers measured in step (d), i.e. the control sample is a positive control.

For the avoidance of doubt, control samples from more than one disease state may be provided in step (c), for example, ≥2, ≥3, ≥4, ≥5, ≥6 or ≥7 different disease states. Step (c) may provide at least two control samples, for example, ≥3, ≥4, ≥5, ≥6, ≥7, ≥8, ≥9, ≥10, ≥15, ≥20, ≥25, ≥50 or ≥100 control samples. Where multiple control samples are provided, they may be of the same type (e.g., all serum or urine samples) or of different types (e.g., serum and urine samples). Preferably the test samples types and control samples types are matched/corresponding.

By “is different to the presence and/or amount in a control sample” we mean or include the presence and/or amount of the one or more biomarker in the test sample differs from that of the one or more control sample (or to predefined reference values representing the same). Preferably the presence and/or amount in the test sample differs from the presence or amount in the one or more control sample (or mean of the control samples) by at least ±5%, for example, at least ±6%, ±7%, ±8%, ±9%, ±10%, ±11%, ±12%, ±13%, ±14%, ±15%, ±16%, ±17%, ±18%, ±19%, ±20%, ±21%, ±22%, ±23%, ±24%, ±25%, ±26%, ±27%, ±28%, ±29%, ±30%, ±31%, ±32%, ±33%, ±34%, ±35%, ±36%, ±37%, ±38%, ±39%, ±40%, ±41%, ±42%, ±43%, ±44%, ±45%, ±41%, ±42%, ±43%, ±44%, ±55%, ±60%, ±65%, ±66%, ±67%, ±68%, ±69%, ±70%, ±71%, ±72%, ±73%, ±74%, ±75%, ±76%, ±77%, ±78%, ±79%, ±80%, ±81%, ±82%, ±83%, ±84%, ±85%, ±86%, ±87%, ±88%, ±89%, ±90%, ±91%, ±92%, ±93%, ±94%, ±95%, ±96%, ±97%, ±98%, ±99%, ±100%, ±125%, ±150%, ±175%, ±200%, ±225%, ±250%, ±275%, ±300%, ±350%, ±400%, ±500% or at least ±1000% of the one or more control sample (e.g., the negative control sample).

Alternatively or additionally, the presence or amount in the test sample differs from the mean presence or amount in the control samples by at least >1 standard deviation from the mean presence or amount in the control samples, for example, ≥1.5, ≥2, ≥3, ≥4, ≥5, ≥6, ≥7, ≥8, ≥9, ≥10, ≥11, ≥12, ≥13, ≥14 or ≥15 standard deviations from the from the mean presence or amount in the control samples. Any suitable means may be used for determining standard deviation (e.g., direct, sum of square, Welford's), however, in one embodiment, standard deviation is determined using the direct method (i.e., the square root of [the sum the squares of the samples minus the mean, divided by the number of samples]).

Alternatively or additionally, by “is different to the presence and/or amount in a control sample” we mean or include that the presence or amount in the test sample does not correlate with the amount in the control sample in a statistically significant manner. By “does not correlate with the amount in the control sample in a statistically significant manner” we mean or include that the presence or amount in the test sample correlates with that of the control sample with a p-value of >0.001, for example, >0.002, >0.003, >0.004, >0.005, >0.01, >0.02, >0.03, >0.04 >0.05, >0.06, >0.07, >0.08, >0.09 or >0.1. Any suitable means for determining p-value known to the skilled person can be used, including z-test, t-test, Student's t-test, f-test, Mann-Whitney U test, Wilcoxon signed-rank test and Pearson's chi-squared test.

Alternatively, as described above, the autoimmune disease-associated disease state may be identified in the event that the presence and/or amount in the test sample of the one or more biomarkers measured in step (b) corresponds to the presence and/or amount in the control sample of the one or more biomarkers measured in step (d).

Thus, the methods of the invention may comprise steps (c)+(d) for either or both a positive and a negative control.

By “corresponds to the presence and/or amount in a control sample” we include that the presence and/or amount is identical to that of a positive control sample; or closer to that of one or more positive control sample than to one or more negative control sample (or to predefined reference values representing the same). Preferably the presence and/or amount is within ±40% of that of the one or more control sample (or mean of the control samples), for example, within ±39%, ±38%, ±37%, ±36%, ±35%, ±34%, ±33%, ±32%, ±31%, ±30%, ±29%, ±28%, ±27%, ±26%, ±25%, ±24%, ±23%, ±22%, ±21%, ±20%, ±19%, ±18%, ±17%, ±16%, ±15%, ±14%, ±13%, ±12%, ±11%, ±10%, ±9%, ±8%, ±7%, ±6%, ±5%, ±4%, ±3%, ±2%, ±1%, ±0.05% or within 0% of the one or more control sample (e.g., the positive control sample).

Alternatively or additionally, the difference in the presence or amount in the test sample is standard deviation from the mean presence or amount in the control samples, for example, ≤4.5, ≤4, ≤3.5, ≤3, ≤2.5, ≤2, ≤1.5, ≤1.4, ≤1.3, ≤1.2, ≤1.1, ≤1, ≤0.9, ≤0.8, ≤0.7, ≤0.6, ≤0.5, ≤0.4, ≤0.3, ≤0.2, ≤0.1 or 0 standard deviations from the from the mean presence or amount in the control samples, provided that the standard deviation ranges for differing and corresponding biomarker expressions do not overlap (e.g., abut, but no not overlap).

Alternatively or additionally, by “corresponds to the presence and/or amount in a control sample” we include that the presence or amount in the test sample correlates with the amount in the control sample in a statistically significant manner. By “correlates with the amount in the control sample in a statistically significant manner” we mean or include that the presence or amount in the test sample correlates with the that of the control sample with a p-value of ≤0.05, for example, ≤0.04, ≤0.03, ≤0.02, ≤0.01, ≤0.005, ≤0.004, ≤0.003, ≤0.002, ≤0.001, ≤0.0005 or ≤0.0001.

Differential expression (up-regulation or down regulation) of biomarkers, or lack thereof, can be determined by any suitable means known to a skilled person. Differential expression is determined to a p value of a least less than 0.05 (p=<0.05), for example, at least <0.04, <0.03, <0.02, <0.01, <0.009, <0.005, <0.001, <0.0001, <0.00001 or at least <0.000001. For example, differential expression may be determined using a support vector machine (SVM).

In one embodiment, the SVM is, or is derived from, the SVM described below in Supplementary Table S4.

It will be appreciated by persons skilled in the art that differential expression may relate to a single biomarker or to multiple biomarkers considered in combination (i.e., as a biomarker signature). Thus, a p value may be associated with a single biomarker or with a group of biomarkers. Indeed, proteins having a differential expression p value of greater than 0.05 when considered individually may nevertheless still be useful as biomarkers in accordance with the invention when their expression levels are considered in combination with one or more other biomarkers.

As exemplified in the accompanying Example, the expression of certain proteins in a tissue, blood, serum or plasma test sample may be indicative of an autoimmune disease in an individual. For example, the relative expression of certain serum proteins in a single test sample may be indicative of the presence of an autoimmune disease in an individual.

In an alternative or additional embodiment, the presence and/or amount in the test sample of the one or more biomarkers measured in step (b) may be compared against predetermined reference values representative of the measurements in step (d) i.e., reference negative and/or positive control values.

As detailed above, the methods of the invention may also comprise measuring, in one or more negative or positive control samples, the presence and/or amount of the one or more biomarkers measured in the test sample in step (b).

For example, one or more negative control samples may be from an individual who was not, at the time the sample was obtained, afflicted with:
(a) an autoimmune disease;
(b) a specific autoimmune disease selected from systemic lupus erythematosus (SLE), rheumatoid arthritis (RA), Sjögren's syndrome (SS) or systemic vasculitis (SV); and/or
(c) any other disease or condition.

Thus, the negative control sample may be obtained from a healthy individual, for example one afflicted with none of (a), (b) or (c) above.

Likewise, one or more positive control samples may be from an individual who, at the time the sample was obtained, was afflicted with an autoimmune disease; and/or any other disease or condition.

In one embodiment of the methods of the invention, the control samples of step (c) are provided from an individual with systemic lupus erythematosus (SLE), rheumatoid arthritis (RA), Sjögren's syndrome (SS) or systemic vasculitis (SV).

In a preferred embodiment of the second aspect of the invention the control sample is provided from an individual with systemic lupus erythematosus. In a preferred embodiment of the third aspect of the invention the control sample is provided from an individual with rheumatoid arthritis. In a preferred embodiment of the fourth aspect of the invention the control sample is provided from an individual with Sjögren's syndrome. In a preferred embodiment of the fifth aspect of the invention the control sample is provided from an individual with systemic vasculitis.

In one embodiment of any of the first to fifth the control samples of step (c) are provided from an individual with systemic lupus erythematosus subtype 1 (SLE-1), systemic lupus erythematosus subtype 2 (SLE-2) or systemic lupus erythematosus subtype 3 (SLE-3). SLE-1 comprises skin and musculoskeletal involvement but lacks serositis, systemic vasculitis and kidney involvement. SLE-2 comprises skin and musculoskeletal involvement, serositis and systemic vasculitis but lacks kidney involvement. SLE-3 comprises skin and musculoskeletal involvement, serositis, systemic vasculitis and SLE glomerulonephritis. SLE-1, SLE-2 and SLE-3 represent mild/absent, moderate and severe SLE disease states, respectively (e.g., see Sturfelt G, Sjoholm A G. Complement components, complement activation, and acute phase response in systemic lupus erythematosus. Int Arch Allergy Appl Immunol 1984; 75:75-83 which is incorporated herein by reference).

In an alternative embodiment, the control samples of step (c) are provided from an individual with rheumatoid arthritis (RA), which may also include extra-articular manifestations, such as nodules, scleritis, Felty's syndrome, neuropathy, pericarditis, pleuritis or glomerulonephritis

In one embodiment, the control samples of step (c) are provided from an individual with primary Sjögren's syndrome. Alternatively, the control samples of step (c) may be provided from an individual with secondary Sjögren's syndrome.

In one embodiment, the control samples of step (c) are provided from an individual with a systemic vasculitis condition, such as antineutrophil cytoplasmic antibody (ANCA) vasculitis. The condition may be selected from MPO systemic vasculitis and/or PR3 systemic vasculitis from patients in active or inactive disease state.

In one embodiment of any of the first to the fifth aspects of the invention, the method is repeated until an autoimmune disease is diagnosed and/or an autoimmune disease associated disease state is determined in the individual using the methods of the present invention and/or conventional clinical methods (i.e., until confirmation of the diagnosis is made).

Thus, steps (a) and (b) may be repeated using a sample from the same individual taken at different time to the original sample tested (or the previous method repetition). Such repeated testing may enable disease progression to be assessed, for example to determine the efficacy of the selected treatment regime and (if appropriate) to select an alternative regime to be adopted.

Thus, in one embodiment, the method is repeated using a test sample taken between 1 day to 104 weeks to the previous test sample(s) used, for example, between 1 week to 100 weeks, 1 week to 90 weeks, 1 week to 80 weeks, 1 week to 70 weeks, 1 week to 60 weeks, 1 week to 50 weeks, 1 week to 40 weeks, 1 week to 30 weeks, 1 week to 20 weeks, 1 week to 10 weeks, 1 week to 9 weeks, 1 week to 8 weeks, 1 week to 7 weeks, 1 week to 6 weeks, 1 week to 5 weeks, 1 week to 4 weeks, 1 week to 3 weeks, or 1 week to 2 weeks.

Alternatively or additionally, the method may be repeated using a test sample taken every period from the group consisting of: 1 day, 2 days, 3 day, 4 days, 5 days, 6 days, 7 days, 10 days, 2 weeks, 3 weeks, 4 weeks, 5 weeks, 6 weeks, 7 weeks, 8 weeks, 9 weeks, 10 weeks, 15 weeks, 20 weeks, 25 weeks, 30 weeks, 35 weeks, 40 weeks, 45 weeks, 50 weeks, 55 weeks, 60 weeks, 65 weeks, 70 weeks, 75 weeks, 80 weeks, 85 weeks, 90 weeks, 95 weeks, 100 weeks, 104, weeks, 105 weeks, 110 weeks, 115 weeks, 120 weeks, 125 weeks and 130 weeks.

Alternatively or additionally, the method may be repeated at least once, for example, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 21 times, 22 times, 23, 24 times or 25 times.

Alternatively or additionally, the method is repeated continuously.

In one preferred embodiment of the methods of the invention, step (a) comprises providing a serum sample from an individual to be tested and/or step (b) comprises measuring in the sample the expression of the protein or polypeptide of the one or more biomarker(s). Thus, a biomarker signature for the sample may be determined at the protein level.

In such an embodiment, step (b) and/or step (d) may be performed using one or more first binding agents capable of binding to a biomarker (i.e., protein) listed in Table 1(A), Table 1(B), Table 1(C), Table 1(D), or Table 1(E). It will be appreciated by persons skilled in the art that the first binding agent may comprise or consist of a single species with specificity for one of the protein biomarkers or a plurality of different species, each with specificity for a different protein biomarker.

In one embodiment, the one or more first binding agents are selected from those listed in Supplementary table S5 and/or Supplementary table S6.

Suitable binding agents (also referred to as binding molecules) can be selected from a library, based on their ability to bind a given target molecule, as discussed below.

In one preferred embodiment, at least one type of the binding agents, and more typically all of the types, may comprise or consist of an antibody or antigen-binding fragment of the same, or a variant thereof.

Methods for the production and use of antibodies are well known in the art, for example see Antibodies: A Laboratory Manual, 1988, Harlow & Lane, Cold Spring Harbor Press, ISBN-13: 978-0879693145, Using Antibodies: A Laboratory Manual, 1998, Harlow & Lane, Cold Spring Harbor Press, ISBN-13: 978-0879695446 and Making and Using Antibodies: A Practical Handbook, 2006, Howard & Kaser, CRC Press, ISBN-13: 978-0849335280 (the disclosures of which are incorporated herein by reference).

Thus, a fragment may contain one or more of the variable heavy (V_{H}) or variable light (V_{L}) domains. For example, the term antibody fragment includes Fab-like molecules (Better et al (1988) Science 240, 1041); Fv molecules (Skerra et al (1988) Science 240, 1038); single-chain Fv (scFv) molecules where the V_{H} and V_{L} partner domains are linked via a flexible oligopeptide (Bird et al (1988) Science 242, 423; Huston et al (1988) Proc. Natl. Acad. Sci. USA 85, 5879) and single domain antibodies (dAbs) comprising isolated V domains (Ward et al (1989) Nature 341, 544).

For example, the binding agent(s) may be scFv molecules, Fabs or the binding domains of immunoglobulin molecules.

The term “antibody variant” includes any synthetic antibodies, recombinant antibodies or antibody hybrids, such as but not limited to, a single-chain antibody molecule produced by phage-display of immunoglobulin light and/or heavy chain variable and/or constant regions, or other immunointeractive molecule capable of binding to an antigen in an immunoassay format that is known to those skilled in the art.

A general review of the techniques involved in the synthesis of antibody fragments which retain their specific binding sites is to be found in Winter & Milstein (1991) Nature 349, 293-299.

Molecular libraries such as antibody libraries (Clackson et al, 1991, Nature 352, 624-628; Marks et al, 1991, J Mol Biol 222(3): 581-97), peptide libraries (Smith, 1985, Science 228(4705): 1315-7), expressed cDNA libraries (Santi et al (2000) J Mol Biol 296(2): 497-508), libraries on other scaffolds than the antibody framework such as affibodies (Gunneriusson et al, 1999, Appl Environ Microbiol 65(9): 4134-40) or libraries based on aptamers (Kenan et al, 1999, Methods Mol Biol 118, 217-31) may be used as a source from which binding molecules that are specific for a given motif are selected for use in the methods of the invention.

Conveniently, the binding agent(s) may be immobilised on a surface (e.g., on a multiwell plate or array); see Example below.

In one embodiment of the methods of the invention, step (b), (d) and/or step (f) is performed using an assay comprising a second binding agent capable of binding to the one or more biomarkers, the second binding agent comprising a detectable moiety. For example, an immobilised (first) binding agent may initially be used to ‘trap’ the protein biomarker on to the surface of a microarray, and then a second binding agent may be used to detect the ‘trapped’ protein.

The second binding agent may be as described above in relation to the (first) binding agent, such as an antibody or antigen-binding fragment thereof.

It will be appreciated by skilled person that the one or more biomarkers (e.g., proteins) in the test sample may be labelled with a detectable moiety, prior to performing step (b). Likewise, the one or more biomarkers in the control sample(s) may be labelled with a detectable moiety. The biomarker(s) may be labelled with a directly or indirectly detectable moiety.

Alternatively, or in addition, the first and/or second binding agents may be labelled with a detectable moiety.

By a “detectable moiety” we include the meaning that the moiety is one which may be detected and the relative amount and/or location of the moiety (for example, the location on an array) determined.

Suitable detectable moieties are well known in the art. For example, the detectable moiety may be selected from the group consisting of: a fluorescent moiety; a luminescent moiety; a chemiluminescent moiety; a radioactive moiety; an enzymatic moiety.

In one preferred embodiment, the detectable moiety is biotin.

In one embodiment, in step (b) and/or step (d) the biotinylated biomarkers are detected using streptavidin labelled with a detectable moiety selected from the group consisting of: a fluorescent moiety; a luminescent moiety; a chemiluminescent moiety; a radioactive moiety; an enzymatic moiety.

Thus, the detectable moiety may be a fluorescent and/or luminescent and/or chemiluminescent moiety which, when exposed to specific conditions, may be detected. For example, a fluorescent moiety may need to be exposed to radiation (i.e., light) at a specific wavelength and intensity to cause excitation of the fluorescent moiety, thereby enabling it to emit detectable fluorescence at a specific wavelength that may be detected.

Alternatively, the detectable moiety may be an enzyme which is capable of converting a (preferably undetectable) substrate into a detectable product that can be visualised and/or detected. Examples of suitable enzymes are discussed in more detail below in relation to, for example, ELISA assays.

In a further alternative, the detectable moiety may be a radioactive atom which is useful in imaging. Suitable radioactive atoms include ⁹⁹mTc and ¹²³I for scintigraphic studies. Other readily detectable moieties include, for example, spin labels for magnetic resonance imaging (MRI) such as ¹²³I again, ¹³¹I, ¹¹¹In, ¹⁹F, ¹³C, ¹⁵N, ¹⁷O, gadolinium, manganese or iron. Clearly, the agent to be detected (such as, for example, the one or more biomarkers in the test sample and/or control sample described herein and/or an antibody molecule for use in detecting a selected protein) must have sufficient of the appropriate atomic isotopes in order for the detectable moiety to be readily detectable.

Preferred assays for detecting serum or plasma proteins include enzyme linked immunosorbent assays (ELISA), radioimmunoassay (RIA), immunoradiometric assays (IRMA) and immunoenzymatic assays (IEMA), including sandwich assays using monoclonal and/or polyclonal antibodies. Exemplary sandwich assays are described by David et al in U.S. Pat. Nos. 4,376,110 and 4,486,530, hereby incorporated by reference. Antibody staining of cells on slides may be used in methods well known in cytology laboratory diagnostic tests, as well known to those skilled in the art.

Conveniently, the assay is an ELISA (Enzyme Linked Immunosorbent Assay) which typically involves the use of enzymes giving a coloured reaction product, usually in solid phase assays. Enzymes such as horseradish peroxidase and phosphatase have been widely employed. A way of amplifying the phosphatase reaction is to use NADP as a substrate to generate NAD which now acts as a coenzyme for a second enzyme system. Pyrophosphatase from Escherichia coli provides a good conjugate because the enzyme is not present in tissues, is stable and gives a good reaction colour. Chemi-luminescent systems based on enzymes such as luciferase can also be used.

ELISA methods are well known in the art, for example see The ELISA Guidebook (Methods in Molecular Biology), 2000, Crowther, Humana Press, ISBN-13: 978-0896037281 (the disclosures of which are incorporated by reference).

Alternatively, conjugation with the vitamin biotin is frequently used since this can readily be detected by its reaction with enzyme-linked avidin or streptavidin to which it binds with great specificity and affinity.

In one embodiment, the detectable moiety is fluorescent moiety (for example an Alexa Fluor dye, e.g. Alexa647).

In one preferred embodiment, step (b) and/or step (d) may be performed using an array.

Arrays per se are well known in the art. Typically, they are formed of a linear or two-dimensional structure having spaced apart (i.e. discrete) regions (“spots”), each having a finite area, formed on the surface of a solid support. An array can also be a bead structure where each bead can be identified by a molecular code or colour code or identified in a continuous flow. Analysis can also be performed sequentially where the sample is passed over a series of spots each adsorbing the class of molecules from the solution. The solid support is typically glass or a polymer, the most commonly used polymers being cellulose, polyacrylamide, nylon, polystyrene, polyvinyl chloride or polypropylene. The solid supports may be in the form of tubes, beads, discs, silicon chips, microplates, polyvinylidene difluoride (PVDF) membrane, nitrocellulose membrane, nylon membrane, other porous membrane, non-porous membrane (e.g. plastic, polymer, perspex, silicon, amongst others), a plurality of polymeric pins, or a plurality of microtitre wells, or any other surface suitable for immobilising proteins, polynucleotides and other suitable molecules and/or conducting an immunoassay. The binding processes are well known in the art and generally consist of cross-linking covalently binding or physically adsorbing a protein molecule, polynucleotide or the like to the solid support. By using well-known techniques, such as contact or non-contact printing, masking or photolithography, the location of each spot can be defined. For reviews see Jenkins, R. E., Pennington, S. R. (2001, Proteomics, 2, 13-29) and Lal et al (2002, Drug Discov Today 15; 7(18 Suppl): S143-9).

Typically, the array is a microarray. By “microarray” we include the meaning of an array of regions having a density of discrete regions of at least about 100/cm², and preferably at least about 1000/cm². The regions in a microarray have typical dimensions, e.g., diameters, in the range of between about 10-250 μm, and are separated from other regions in the array by about the same distance. The array may also be a macroarray or a nanoarray.

Once suitable binding molecules (discussed above) have been identified and isolated, the skilled person can manufacture an array using methods well known in the art of molecular biology.

Examples of array formats are described below in the Example and in; e.g., Steinhauer et al., 2002; Wingren and Borrebaeck, 2008; Wingren et al., 2005, Delfani et al., 2016 (the disclosure of which are incorporated herein by reference).

Thus, in an exemplary embodiment the method comprises:
(i) labelling biomarkers present in the sample (e.g., serum) with biotin;
(ii) contacting the biotin-labelled proteins with an array comprising a plurality of scFv immobilised at discrete locations on its surface, the scFv having specificity for one or more of the proteins in Table 1(A), Table 1(B), Table 1(C), Table 1(D) and/or Table 1(E);
(iii) contacting the biotin-labelled proteins (immobilised on the surface-bound scFv) with a streptavidin conjugate comprising a fluorescent dye; and
(iv) detecting the presence of the dye at discrete locations on the array surface
   wherein the expression of the dye on the array surface is indicative of the expression of a biomarker from Table 1(A), Table 1(B), Table 1(C), Table 1(D), or Table 1(E) in the sample.

In an alternative embodiment, step (b) and/or step (d) comprises measuring the expression of a nucleic acid molecule encoding the one or more biomarkers.

The nucleic acid molecule may be a gene expression intermediate or derivative thereof, such as a mRNA or cDNA.

Thus, measuring the expression of the one or more biomarker(s) in step (b) and/or step (d) may be performed using a method selected from the group consisting of Southern hybridisation, Northern hybridisation, polymerase chain reaction (PCR), reverse transcriptase PCR (RT-PCR), quantitative real-time PCR (qRT-PCR), nanoarray, microarray, macroarray, autoradiography and in situ hybridisation.

For example, measuring the expression of the one or more biomarker(s) in step (b) and/or step (d) may be performed using one or more binding moieties, each individually capable of binding selectively to a nucleic acid molecule encoding one of the biomarkers identified in Table 1(A), Table 1(B), Table 1(C), Table 1(D) or Table 1(E).

Conveniently, the one or more binding moieties each comprise or consist of a nucleic acid molecule, such as DNA, RNA, PNA, LNA, GNA, TNA or PMO.

Advantageously, the one or more binding moieties are 5 to 100 nucleotides in length. For example, 15 to 35 nucleotides in length.

It will be appreciated that the nucleic acid-based binding moieties may comprise a detectable moiety.

Thus, the detectable moiety may be selected from the group consisting of: a fluorescent moiety; a luminescent moiety; a chemiluminescent moiety; a radioactive moiety (for example, a radioactive atom); or an enzymatic moiety.

Alternatively or additionally, the detectable moiety may comprise or consist of a radioactive atom, for example selected from the group consisting of technetium-99m, iodine-123, iodine-125, iodine-131, indium-111, fluorine-19, carbon-13, nitrogen-15, oxygen-17, phosphorus-32, sulphur-35, deuterium, tritium, rhenium-186, rhenium-188 and yttrium-90.

Alternatively or additionally, the detectable moiety of the binding moiety may be a fluorescent moiety.

In a further embodiment, the nucleic acid molecule is a circulating tumour DNA molecule (ctDNA).

Methods suitable for detecting ctDNA are now well-established; for example, see Lewis et al., 2016, World J Gastroenterol. 22(32): 7175-7185, and references cited therein (the disclosures of which are incorporated herein by reference).

In one embodiment, expression of the one or more biomarker(s) in step (b) is determined using a DNA microarray.

In one embodiment, the methods may be performed using the methods for detecting and/or quantifying one or more biomarker(s) in a biological sample as described in PCT/EP2019/052105 filed on 29 Jan. 2019.

In one embodiment, the sample provided in step (a) (and/or in step (c)) may be selected from the group consisting of unfractionated blood, plasma, serum, tissue fluid, milk, bile, synovial fluid, and urine.

Conveniently, the sample provided in step (a) and/or (c) is unfractionated blood, plasma, or serum. In one embodiment, the sample provided in step (a) and/or (c) is serum.

By appropriate selection of some or all of the biomarkers in Table 1(A), 1(B), 1(C), 1(D) and/or 1(E), optionally in conjunction with one or more further biomarkers, the methods of the invention exhibit high predictive accuracy for diagnosis of an autoimmune disease, including SLE, SV, SS and RA.

Thus, the predictive accuracy of the method, as determined by an ROC AUC value, may be at least 0.50, for example at least 0.55, 0.60, 0.65, 0.70, 0.75, 0.80, 0.85, 0.90, 0.95, 0.96, 0.97, 0.98 or at least 0.99.

Thus, in one embodiment, the predictive accuracy of the method, as determined by an ROC AUC value, is at least 0.70.

In the methods of the invention, the ‘raw’ data obtained in step (b) (and/or in step (d)) undergoes one or more analysis steps before a diagnosis is reached. For example, the raw data may need to be standardised against one or more control values (i.e., normalised).

Typically, diagnosis is performed using a support vector machine (SVM), such as those available from http://cran.r-project.org/web/packages/e1071/index.html (e.g. e1071 1.5-24). However, any other suitable means may also be used.

Support vector machines (SVMs) are a set of related supervised learning methods used for classification and regression. Given a set of training examples, each marked as belonging to one of two categories, an SVM training algorithm builds a model that predicts whether a new example falls into one category or the other. Intuitively, an SVM model is a representation of the examples as points in space, mapped so that the examples of the separate categories are divided by a clear gap that is as wide as possible. New examples are then mapped into that same space and predicted to belong to a category based on which side of the gap they fall on.

More formally, a support vector machine constructs a hyperplane or set of hyperplanes in a high or infinite dimensional space, which can be used for classification, regression or other tasks. Intuitively, a good separation is achieved by the hyperplane that has the largest distance to the nearest training data points of any class (so-called functional margin), since in general the larger the margin the lower the generalization error of the classifier. For more information on SVMs, see for example, Burges, 1998, Data Mining and Knowledge Discovery, 2:121-167.

In one embodiment of the invention, the SVM is ‘trained’ prior to performing the methods of the invention using biomarker profiles from individuals with known disease status (for example, individuals known to have an autoimmune disease or individuals known to be healthy). By running such training samples, the SVM is able to learn what biomarker profiles are associated with an autoimmune disease. Once the training process is complete, the SVM is then able to determine whether or not the biomarker sample tested is from an individual with an autoimmune disease.

However, this training procedure can be by-passed by pre-programming the SVM with the necessary training parameters. For example, diagnoses can be performed according to the known SVM parameters using the SVM algorithm detailed in Supplementary Table S4 below, based on the measurement of any or all of the biomarkers listed in Table 1(A), Table 1(B), Table 1(C), Table 1(D) and/or Table 1(E).

It will be appreciated by skilled persons that suitable SVM parameters can be determined for any combination of the biomarkers listed in Tables 1(A), 1(B), 1(C), 1(D) and/or 1(E) by training an SVM machine with the appropriate selection of data (i.e. biomarker measurements from individuals with known autoimmune disease status). Alternatively, the data of the Examples and figures may be used to determine a particular autoimmune disease-associated disease state according to any other suitable statistical method known in the art.

Preferably, the method of the invention has an accuracy of at least 60%, for example 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% accuracy.

Preferably, the method of the invention has a sensitivity of at least 60%, for example 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sensitivity.

Preferably, the method of the invention has a specificity of at least 60%, for example 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% specificity.

By “accuracy” we mean the proportion of correct outcomes of a method, by “sensitivity” we mean the proportion of all autoimmune disease positive samples that are correctly classified as positives, and by “specificity” we mean the proportion of all autoimmune disease negative samples that are correctly classified as negatives.

Signal intensities may be quantified using any suitable means known to the skilled person, for example using Array-Pro (Media Cybernetics). Signal intensity data may be normalised (i.e., to adjust technical variation). Normalisation may be performed using any suitable method known to the skilled person. Alternatively or additionally, data are normalised using the empirical Bayes algorithm ComBat (Johnson et al., 2007).

Further statistical analysis of the refined data may be performed using methods well-known in the art, such as PCA, q-value calculation by ANOVA, and/or fold change calculation in Qlucore Omics Explorer.

As described above, a first (‘training’) data set may be used to identify a combination of biomarkers, e.g. from Table 1(A), Table 1(B), Table 1(C), Table 1(D), or Table 1(E), to serve as a biomarker signature for the diagnosis of an autoimmune disease. Mathematical analysis of the training data set may be performed using known algorithms (such as a backward elimination, or BE, algorithm) to determine the most suitable biomarker signatures. The predictive accuracy of a given biomarker combination (signature) can then be verified against a new (‘verification’) data set. Such methodology is described in detail in the Example.

It will be appreciated by persons skilled in the art that the individual(s) tested may be of any ethnicity or geographic origin. Alternatively, the individual(s) tested may be of a defined sub-population, e.g., based on ethnicity and/or geographic origin. For example, the individual(s) tested may be Caucasian and/or Chinese (e.g., Han ethnicity).

In one embodiment of any of the first to the fifth aspects of the invention, a diagnosis in a patient of an autoimmune disease is subsequently made using one or more diagnostic tests for an autoimmune disease.

Suitable conventional clinical methods are well known in the art. For example, diagnostic tests for an autoimmune disease may include auto antibody tests such as Anti Nuclear Antibody test (ANA), Anti-Double Stranded DNA (anti-dsDNA), Antineutrophil Cytoplasmic antibodies (ANCA), Cyclic Citrullinated Peptide Antibodies (CCP), Rheumatoid Factor (RF), Extractable Nuclear Antigen Antibodies (e.g. anti-SS-A (Ro) and anti-SS-B (La), anti-sm, anti-RNP, anti-Jo-1, Scl-70), antihistone antibodies and AntiCentromere Antibodies (ACA) or complement analysis (C3, C4).

In one embodiment of any of the first to fifth aspects of the invention, the methods comprise, in the event that the individual is diagnosed with an autoimmune disease, the additional step (g) of administering to the individual a therapy for said autoimmune disease.

Optionally the autoimmune disease therapy is selected from the group consisting of: Nonsteroidal anti-inflammatory drugs (NSAID) such as Ibuprofen and Naproxen; immune-suppressing drugs such as Corticosteroids; synthetic DMARDs (such as Methotrexate, cyclophosphoamide); and Biologicals (such as TNF-inhibitors, IL-inhibitors); and combinations thereof.

A further aspect of the invention provides an array for diagnosing or detecting an autoimmune disease in an individual comprising one or more agents (such as any of the above-described binding agents) suitable for measuring the presence and/or amount of one or more biomarkers selected from the group defined in Table 1(A), Table 1(B), Table 1(C), Table 1(D), and/or Table 1(E).

Thus, the array is suitable for performing a method according to any one of the first to fifth aspects of the invention.

The array comprises one or more binding agents capable (individually or collectively) of binding to one or more of the biomarkers defined in Table 1(A), Table 1(B), Table 1(C), Table 1(D) and/or Table 1(E), either at the protein level or the nucleic acid level.

In one preferred embodiment, the array comprises one or more antibodies, or antigen-binding fragments thereof, capable (individually or collectively) of binding to one or more of the biomarkers defined in Table 1(A), Table 1(B), Table 1(C), Table 1(D) and/or Table 1(E), at the protein level. For example, the array may comprise scFv molecules capable (collectively) of binding to all of the biomarkers defined in Table 1(A) at the protein level.

It will be appreciated that the array may comprise one or more positive and/or negative control samples. For example, conveniently the array comprises bovine serum albumin as a positive control sample and/or phosphate-buffered saline as a negative control sample.

In one embodiment, the array comprises agents capable of binding to all of the biomarkers defined in any one of: Table 1(A), Table 1(B), Table 1(C), Table 1(D) or Table 1(E), In another embodiment, the array comprises agents capable of binding to one or more of the biomarkers defined in any one of: Table 1(A), Table 1(B), Table 1(C), Table 1(D) or Table 1(E), e.g. agents capable of binding to any of the particular combinations of the biomarkers defined in Table 1(A) as described in the first aspect.

Advantageously, the array comprises antibodies, or antigen-binding fragments thereof, capable of binding to all of the biomarkers at the protein level.

Advantageously, the array comprises agents capable of binding to all of the biomarkers at the mRNA and/or DNA level.

A further aspect of the invention provides the use of one or more biomarkers selected from the group defined in Table 1(A), Table 1(B), Table 1(C), Table 1(D) and/or Table 1(E) as biomarkers for diagnosing or detecting an autoimmune disease in an individual.

Optionally, the autoimmune disease is selected from systemic lupus erythematosus (SLE), rheumatoid arthritis (RA), Sjögren's syndrome (SS) or systemic vasculitis (SV).

In one embodiment all of the biomarkers defined in Table 1(A), Table 1(B), Table 1(C), Table 1(D) and/or Table 1(E) are used as biomarkers for diagnosing or detecting an autoimmune disease in an individual. Optionally, the autoimmune disease is selected from systemic lupus erythematosus (SLE), rheumatoid arthritis (RA), Sjögren's syndrome (SS) or systemic vasculitis (SV).

A further aspect of the invention provides kit for diagnosing or detecting an autoimmune disease in an individual comprising:
i) one or more first binding agents capable of binding to one or more biomarker selected from the biomarkers of Table 1(A), Table 1(B), Table 1(C), Table 1(D) and/or Table 1(E);
ii) (optionally) instructions for performing the method as defined in any of the first to fifth aspects of the invention.

A further aspect of the invention provides a kit for determining the presence of, or risk of having, an autoimmune disease in an individual comprising:
(a) an array according to the invention, or components for making the same; and
(b) instructions for performing the method as defined above (e.g., in any of the first to the fifth aspects of the invention).

A further aspect of the invention provides a use of one or more binding moieties to a biomarker as described herein (e.g. in Table 1(A)) in the preparation of a kit for diagnosing or determining an autoimmune disease-associated disease state in an individual. Thus, multiple different binding moieties may be used, each targeted to a different biomarker, in the preparation of such as kit. In one embodiment, the binding moiety is an antibody or antigen-binding fragment thereof (e.g. scFv), as described herein.

A further aspect of the invention provides a method of treating an autoimmune disease in an individual comprising the steps of:
(a) diagnosing an individual with an autoimmune disease using a method according to any one of the first to fifth aspects of the invention; and
(b) providing the individual with a therapy to treating said autoimmune disease.

For example, the autoimmune disease therapy may be selected from SLE, RA, SS or SV.

A further aspect of the invention provides a computer program for operating the methods the invention, for example, for interpreting the expression data of step (c) (and subsequent expression measurement steps) and thereby diagnosing or determining an autoimmune disease-associated disease state. The computer program may be a programmed SVM. The computer program may be recorded on a suitable computer-readable carrier known to persons skilled in the art. Suitable computer-readable-carriers may include compact discs (including CD-ROMs, DVDs, Blu-ray and the like), floppy discs, flash memory drives, ROM or hard disc drives. The computer program may be installed on a computer suitable for executing the computer program.

Preferred, non-limiting examples which embody certain aspects of the invention will now be described, with reference to the following figures:

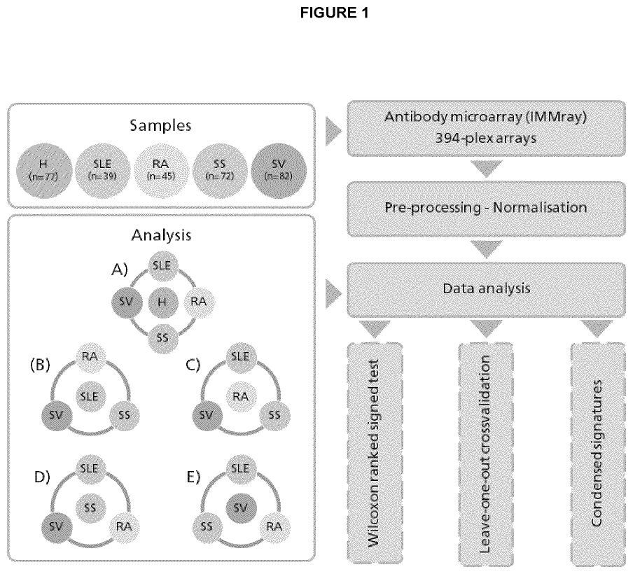

FIG. 1 shows a schematic outline of the antibody microarray process, applied on serum samples from Systemic Lupus Erythematosus (SLE), Rheumatoid Arthritis (RA), Sjögren syndrome (SS), Systemic Vasculitis (SV) and healthy controls (H). For each analysis (Wilcoxon, leave-one-out cross validation and signature development) each group was set against the remaining samples, i.e. (A) H versus SLE+RA+SS+SV (B) SLE versus RA+SS+SV (C) RA versus SLE+SS+SV (D) SS versus SLE+RA+SV and (E) SV versus SLE+RA+SS.

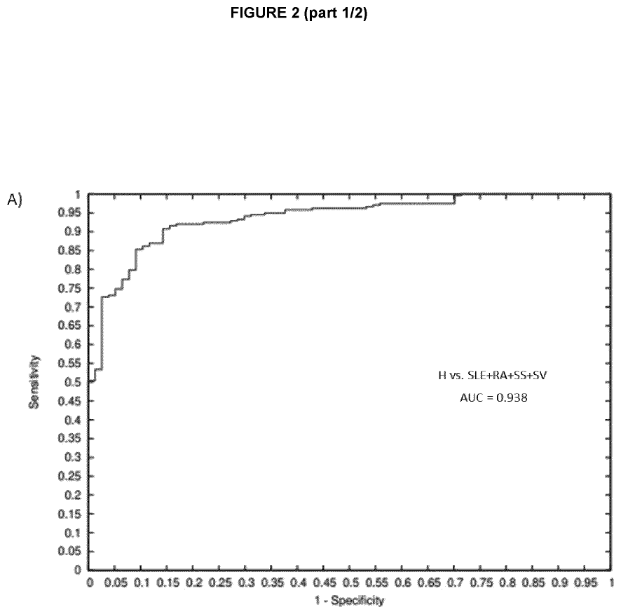

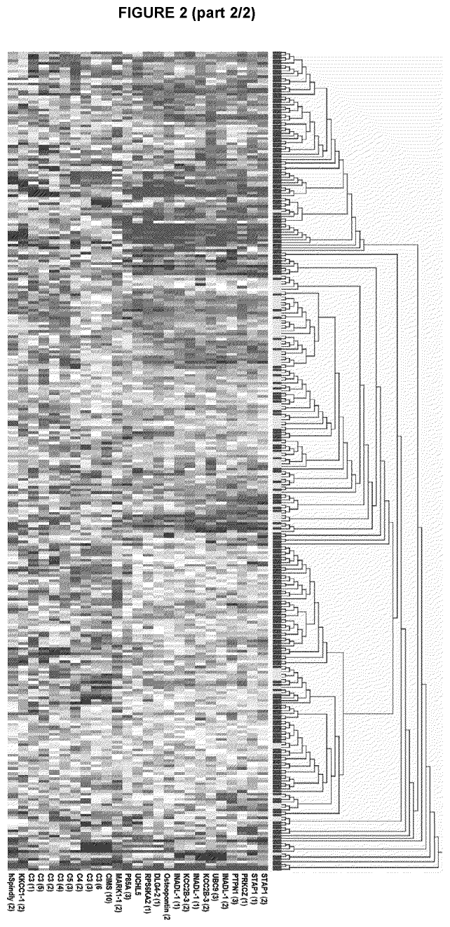

FIG. 2: (A) ROC-curve including AUC-value generated from leave-one-out cross validation analysis on healthy versus autoimmune diseases (SLE, RA, SS and SV). (B) Heatmap from supervised analysis including the top 25 differentially expressed analytes (Wilcoxon analysis q<0.05) between healthy (light bars) and autoimmune diseases (dark bars) which include SLE, RA, SS and SV. Individual clone suffixes are shown in brackets.

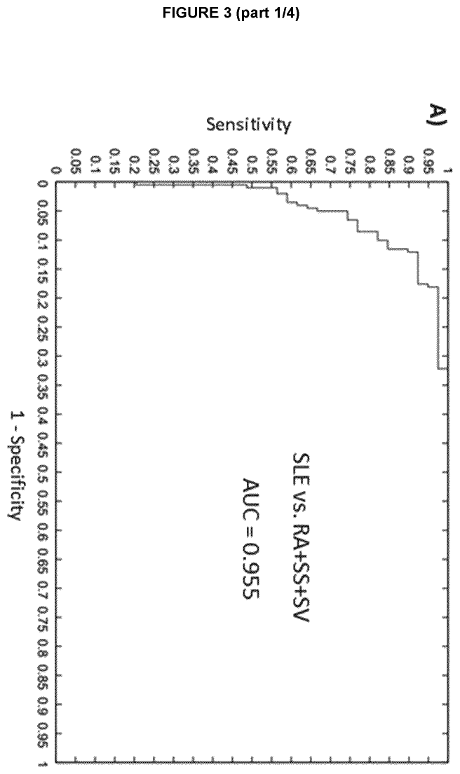

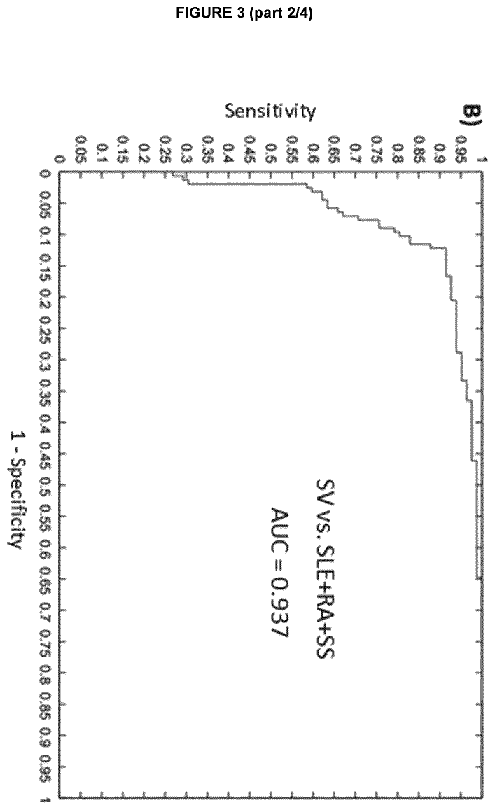

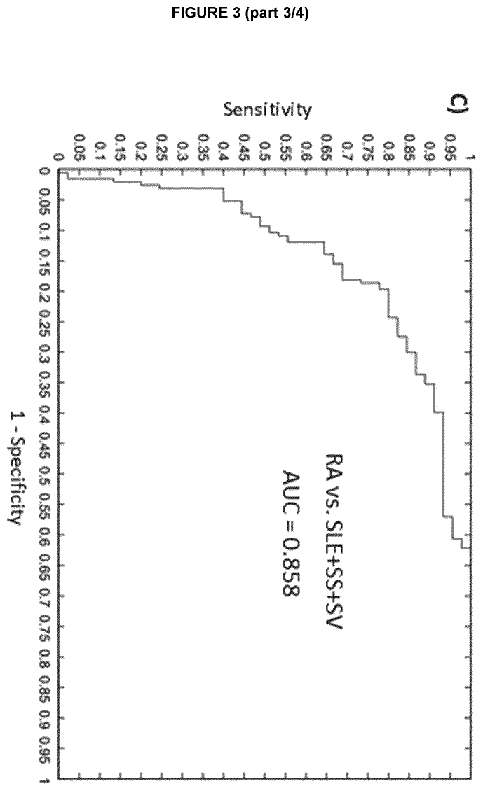

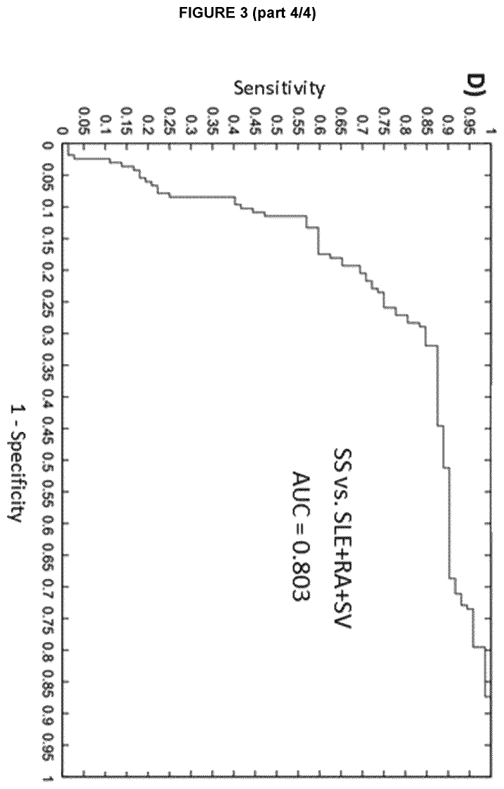

FIG. 3: ROC-curves with AUC-values generated from LOO CV analysis, representing (A) SLE vs. RA+SS+SV (B) SV vs. SLE+RA+SS (C) RA vs. SLE+SS+SV (D) and SS vs. SLE+RA+SV.

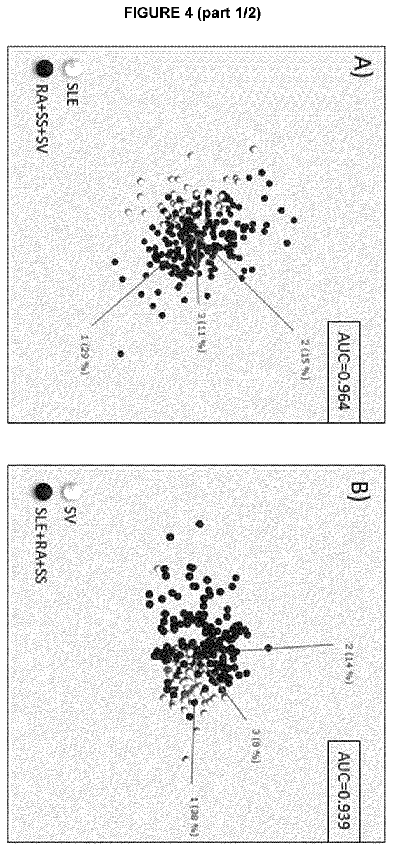

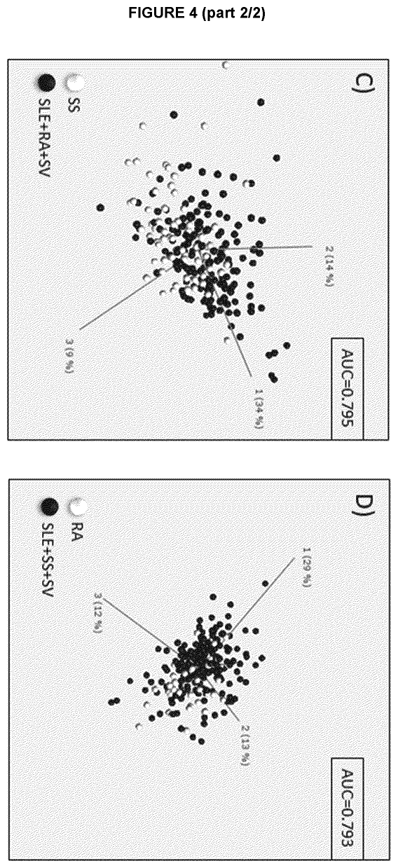

FIG. 4: PCA plots of supervised analysis based on 40-plex biomarker panels representing SLE (A) SV (B) SS (C) and RA (D).

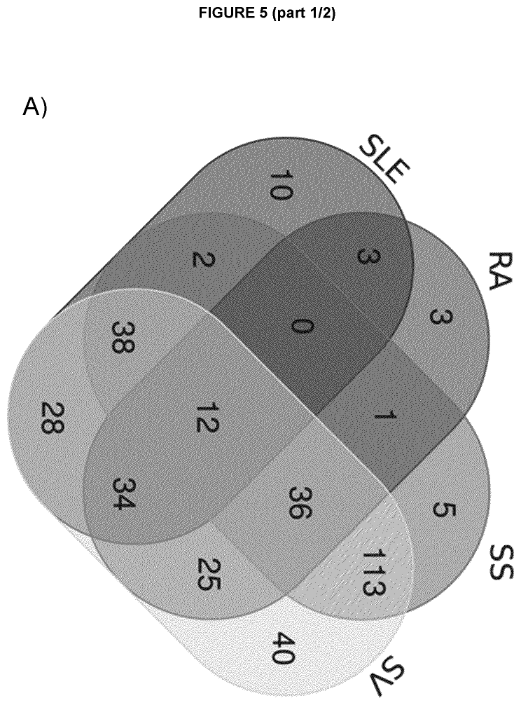

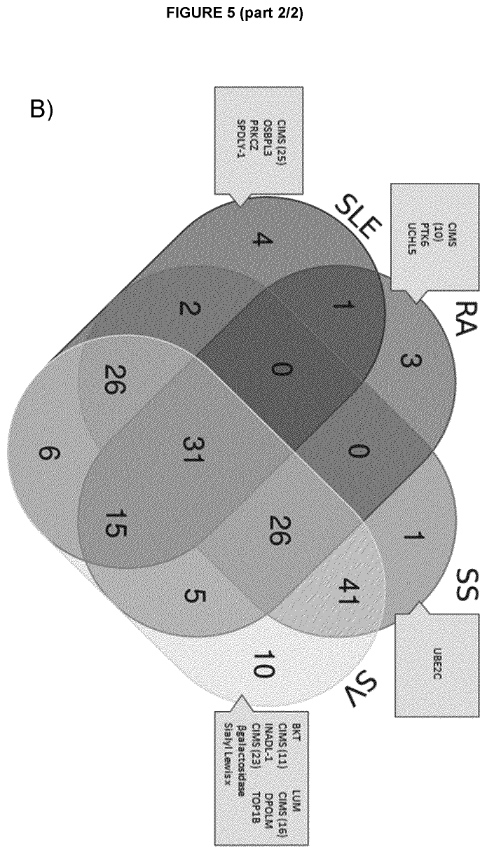

FIG. 5: Venn diagrams representing the overlap of variables generated from differential analysis (Wilcoxon signed rank test, q<0.05) for SLE, RA, SS and SV. Since an analyte may be targeted by more than one antibody, diagram (A) represents the overlap of antibodies whereas (B) represents the overlap on an analyte level. Disease specific analytes are outlined in diagram (B). Diagram created at http://bioinformatics.psb.ugent.be/webtools/Venn/

### EXAMPLE

### Introduction

### Objective

Early and correct diagnosis of autoimmune diseases (AID) pose a clinical challenge due to the multifaceted nature of symptoms which also may change over time. The aim of this study was to perform protein expression profiling of the four systemic AIDs: Systemic Lupus Erythematosus (SLE); Systemic Vasculitis (SV), e.g. ANCA associated Vasculitis; Rheumatoid Arthritis (RA); and Sjögrens Syndrome (SS), and healthy controls and to identify candidate biomarker signatures for differential classification.

### Method

A total of 316 serum samples collected from patients diagnosed with SLE, RA, SS, SV and healthy controls, were analysed using 394-plex recombinant antibody microarrays. Differential protein expression profiling was performed using Wilcoxon rank sum test and condensed biomarker panels were identified using advanced bioinformatics and state-of-the art classification algorithms to pinpoint signatures reflecting disease.

### Results

In this study we were able to classify individual AIDs with high accuracy, as demonstrated by ROC Area Under the Curve (ROC AUC) values ranging between 0.955 to 0.803. In addition, the group of AIDs could be separated from healthy controls at a ROC AUC-value of 0.938. Disease specific candidate biomarker signature as well as a general autoimmune signature were identified, including several deregulated analytes.

### Conclusion

This study supports the rationale of using multiplexed affinity-based technologies to reflect the biological complexity of autoimmune diseases. A multiplexed approach for decoding multifactorial, complex diseases such as autoimmune diseases, will play a significant role for future diagnostic purposes, essential to prevent severe organ and tissue related damage.

### Materials and Methods

### Clinical Samples

This retrospective study included a total of 316 serum samples collected from healthy controls (n=77) and patients diagnosed with a systemic autoimmune disease (n=239). All samples were collected from Department of Rheumatology Skåne University Hospital (Malmo or Lund). Patients were diagnosed with either SLE (n=39), RA (n=45), SS (n=73) or SV (n=82) and considered, according to their specific clinical criteria's, to be in an active disease when samples were collected. For SLE patients, disease activity was defined using the SLEDAI-2000-score (19) (mean score 7, range 1-19), and all RA patients demonstrated elevated CRP levels (mean 31 (13-55) mg/L). ANCA-specificity in SV patients were defined according to MPO+/− or PR3+/− status and all Sjögren samples were collected from patients that fulfilled the 2002 American-European Consensus Group Criteria (20) for primary SS. As controls, serum samples from healthy individuals with no previous history of autoimmune disease were used. Within the AID cohort mean age was 59 years and the female to male ratio 168:82 whereas the mean age in healthy controls was 60 years and a female to male ratio of 66:11 (Table 3). Ethical approval for the study was granted by the regional ethics review board in Lund, Sweden.

TABLE 3
Clinical data of patients included in the study

| | | | | | Healthy | |
|---|---|---|---|---|---|---|
| | Autoimmune diseases | | | | controls | |
| Parameter | SLE | RA | SS | SV | H | Total |
| No. of samples | n = 39 | n = 45 | n = 73 | n = 82 | n = 77 | n = 316 |
| Female:male ratio | 33:6 | 32:13 | 71:2 | 32:50 | 66:11 | 234:83 |
| Mean age years | 51 (29- | 65 (38- | 61 (24- | 60 (11- | 50 | 60 (11- |
| (range) | 77) | 85) | 85) | 83) | (18-81) | 85) |
| Disease specific | | | | | | |
| variables | | | | | | |
| SLE | | | | | | |
| SLEDAI mean | 7 | | | | | |
| (range) | (1-19) | | | | | |
| RA | | | | | | |
| CRP (mg/L) | | 31 | | | | |
| | | (13-55) | | | | |
| SV | | | | | | |
| ANCA | | | | | | |
| phenotype | | | | | | |
| MPO | | | | 40 | | |
| (active/inactive) | | | | (20/20) | | |
| PR3 | | | | 42 | | |
| (active/inactive) | | | | (20/22) | | |

### Antibody Microarray Production and Analysis

A total of 394 recombinant scFv antibodies were selected from in-house designed large phage display libraries (21, 22) (Supplementary Table S1). Of these, 379 of the scFv antibodies were directed against 161 (mainly immunoregulatory) antigens. The remaining 15 scFv antibodies were directed against 15 short amino acid motifs (4-6 amino acids long), denoted CIMS antibodies. For some analytes more than one scFv antibody clone (2-9) targeting different epitopes, were chosen to minimize the risk of impaired antibody activity followed by epitope masking during sample labelling process. All scFv antibodies were produced, according to standardized protocols, in 15 mL E. coli cultures and purified to from the cell periplasmic space using the MagneHis™ Protein Purification system (Promega, Madison, Wis., USA) and a KingFisher96 robot (Thermo Fisher Scientific. Waltham, Mass., USA). Buffer exchange to PBS was performed using a Zeba™ 96-well desalt spin plate (Pierce) and concentration and purity of the scFvs was determined using Nanodrop at 280 nm (NanoDrop Technologies, Wilmington, USA) and 10% SDS-PAGE (InVitrogen, Carlsbad, Ca, USA). Production of 26×28 subarrays were generated by a noncontact printer (SciFlexarrayer S11, Scenion, Berlin, Germany). Briefly described, single droplets (300 pL) of scFv antibody solutions, PBS (blank) or biotinylated BSA (position marker), were printed on Blank Polymer Maxisorp slides (NUNC A/S, Roskilde, Denmark) and allowed to absorb to the surface. Antibody microarrays were analysed as previously described (23). In brief, biotinylated samples were added to individual subarrays, and bound proteins were detected using Alexa-647 labelled streptavidin. Slides were scanned at 635 nm using the LS Reloaded™ laser scanner (Tecan) at a fixed laser scanning setting of 150% PMT gain.

### Data Pre-Processing

Data pre-processing were performed as described. In brief, spot signal intensities were quantified using the Immunovia Quant™ software, v1.0 (Immunovia AB, Lund, Sweden). Signal intensities with local background subtraction were used for data analysis. Each data point represented the mean value of three technical replicate spots, unless any replicate CV exceeded 15%, in which case the worst performing replicate was eliminated and the average value of the two remaining replicates were used instead. The data was normalised using a two-step strategy. First, the data was normalised according to day-to-day variation using the “subtract by group mean” approach as previously described (24, 25). In the second step, a modified semi-global normalisation was used to minimize any array-to-array variations. In this approach 15% of the antibodies displaying the lowest CV-values over all samples were identified and used to calculate a scaling factor as previously described (26, 27). Quality control and visualization of potential outliers were performed using the Qlucore Omice Explorer 3.1 software (Qlucore AB, Lund, Sweden).

### Data Analysis

A schematic outline of the analysis process is demonstrated in FIG. 1. For differential protein expression analysis, leave-one-out cross-validation and subsequent signature development, one group (H, SLE, RA, SS or SV) was set against the remaining groups. Analysis 1A in FIG. 1 refer to identification of a general autoimmune signature where healthy controls (H) was set against the autoimmune diseases, meaning H versus SLE+RA+SS+SV. When performing analysis within the AID group (FIG. 1B-E) each individual disease was set against the group of the remaining three diseases as described (B) SLE versus RA+SS+SV (C) RA versus SLE+SS+SV (D) SS versus SLE+RA+SV and (E) SV vs. SLE+RA+SS.

Significantly up- or down-regulated proteins were identified using Wilcoxon signed-rank test (q<0.05) and p-values were adjusted with the Benjamini and Hochberg method (28). Venn diagram including differentially expressed analytes was created at http://bioinformatics.psb.ugent.be/webtools/Venn/. For supervised classification analysis a linear support vector machine (SVM) combined with a leave-one-out classification algorithm was used to evaluate the predictive performance of a model. In the LOO CV procedure one sample was removed, and the remaining samples were used to train the model. The left-out sample was then used to test the model and the process was repeated until every sample had been used as a test sample. A decision values for each excluded sample was thus generated, corresponding to the distance to the hyper plane and a receiver operating characteristic (ROC) curve was constructed. The area under the curve (AUC) was then calculated and used as a measure of the prediction performance of the classifier.

To define a condensed biomarker signature for the differential profiling analysis a ranking procedure combined with two levels of K-fold cross validation loops were used. In short, the outer K-fold cross validation loop was used to test a condensed biomarker signature of a given length and the inner loop was used to define a ranking of the antibodies. The final condensed biomarker signature, of a given size, was then assembled using all ranking lists analyzed in the outer loop. For more details see supplemental information below.

### Results

The aim of this study was to perform differential protein expression profiling of autoimmune diseases and healthy controls and to identify condensed biomarker signatures for disease classification. To this end, a total of 316 serum samples, collected from healthy controls (n=77) and patients diagnosed with SLE (n=39), RA (n=45), SV (n=82) or SS (n=73) were analysed on 394-plex antibody microarrays. One sample collected from a patient with Sjögren syndrome was removed from analysis due to technical reasons. One antibody, targeting Keratin-19, was failed during printing process and removed from further analysis, though two clones targeting the same antigen remained. Altogether, a total of 315 samples and 393 antibodies were used for final data analysis, differential profiling, and signature development. Visualization of the data set in Qlucore™ revealed no differences in relation to array block, sample labelling day, assay day or scanning positions, suggesting that eventual technical differences had successfully been removed during normalisation.

### Differential Protein Expression Profiling of Healthy and Autoimmune Serum Samples

In a first step of analysis we wanted to investigate if a signature reflecting AID (including SLE, RA, SS and SV) could be identified. Altogether, we were able to demonstrate that AID could be separated from healthy controls and that a biomarker signature, indicative for AID could indeed be identified. This was done by using SVM analysis combined with a leave-one-out cross validation, including all antibodies (n=393), which demonstrated that AIDs could be separated from healthy controls with a ROC AUC-value of 0.938 (FIG. 2A). Since LOO CV analysis utilizes all antibodies for classification, we wanted to investigate if healthy and autoimmune samples still could be classified using a smaller set of antibodies. Using a ranking procedure (see method section), the 40 best performing antibodies were selected (Supplementary table S2), able to classify AID and healthy controls by a predictive AUC-value of 0.928. These results clearly show that AIDs can be differentiated from healthy controls using a protein signature which paves the way for a diagnostic test of AIDs.

Next, we were interested in which analytes were deregulated among the AIDs. By using Wilcoxon signed rank test, a total of 77 analytes, targeted by 114 antibodies were found to be differentially expressed (q<0.05) between AIDs and healthy controls. Among the upregulated some of the most interesting included antibodies targeting Apolipoprotein A1, IL-6, IL-12, TNF-α, IL-16, Osteopontin, PRKCZ and DLG4, whereas antibodies targeting C3, IL-4, VEGF, KKCC1-1 and SPDLY-1 were found among the downregulated. A heatmap including the top 25 antibodies and their corresponding analytes revealed some separation of the two groups (FIG. 2B, Supplementary data table S3). Supported by the fact that separation of AID from healthy controls could be achieved using two different approaches, though Wilcoxon signed rank test is a nonparametric test and rely on multiple testing, whereas the K-fold cross validation is an algorithm within machine learning to estimate the prediction error, we compared the lists including the top 25 antibodies with the 40-plex panel. Some overlap could be observed including antibodies targeting the analytes C3, C4, RPS6KA2, KCC2B-3C5 and UBC9. Altogether, these results indicated that a general AID signature is present, involving upregulation of several analytes with immunoregulatory functions.

### Differential Protein Expression Profiling of SLE, RA, SS and SV

Considering that many autoimmune diseases display similar symptoms, making clinical diagnosis challenging, we turned our focus towards the AID group for protein expression profiling analysis (FIG. 1 analysis B-E). Herein, a total of four setups was performed as followed (B) SLE vs. RA+SS+SV (C) RA vs. SLE+SS+SV (D) SS vs. SLE+RA+SV and (E) SV vs. SLE+RA+SS. Leave-one-out cross validation analysis, showed that classification of respectively AID-type could be achieved at high accuracies as presented by ROC AUC values ranging from 0.955 at the highest to 0.803 as the lowest (FIG. 3). The best separation was achieved for SLE with a ROC AUC-value of 0.955 (FIG. 3A) followed by SV and RA which were classified at ROC AUC-values of 0.937 (FIG. 3B) and 0.858 (FIG. 3C), respectively whereas SS demonstrated a ROC AUC-value of 0.803 (FIG. 3D).

Again, we were interested in if the different groups could be separated using shorter biomarker signatures. Condensed biomarker signatures for SLE, RA, SS and SV respectively were identified using the same procedure as previous (Supplementary data table S2, B-E). Herein, using the disease-specific signatures, SLE was again found to be classified with highest accuracy (AUC=0.964) followed by SV (AUC=0.939) SS (AUC=0.795) and RA (AUC=0.793), as presented by PCA-plots in FIG. 4. A closer look at these four diseases specific signatures, revealed that antibodies targeting analytes such as C3, C4, Apolipoprotein A1 and Factor B were present on more than one list. However, analytes unique for each signature was also identified, such as Lewis x and TNF-α in SLE, PRKCZ and PTK6 in RA, IL-8 and RANTES in SS and C1q and IL-18 in SV, which could indicate the presence of disease specific markers. Altogether, by applying 394-plex antibody microarrays interfaced with stringent data analysis, 40-plex antibody signatures capable of classifying the autoimmune diseases SLE, RA, SS and SV at high predictive powers, were pinpointed.

To further explore the serum proteomes of SLE, RA, SS and SV, differentially expressed analytes for respective disease type were identified (Wilcoxon. q<0.05) (Supplemental table S3 B-E). In total, the highest number of differentially expressed analytes was found for SV (n=326 antibodies targeting 160 analytes) followed in decreasing order by SS (n=207 antibodies targeting 127 analytes), SLE (n=127 antibodies targeting 85 analytes) and RA (n=114 antibodies targeting 81 analytes). Considering the complexity of underlying molecular alterations in AID and that both common as disease specific alteration would be of interest, we investigated the amount of overlap of analytes. Firstly, we investigated the overlap based on an antibody level, i.e. relating to the specific clones, irrespective of which analytes they targeted. This revealed a major overlap (FIG. 5A) which was not surprising considering the high number of antibodies generated from differential analysis. Secondly, we focused on an analyte level, which from a biological perspective would be more interesting. As seen in the Venn diagram (FIG. 5B) a few disease specific analytes were observed, of which OSBPL3, PRKCZ, SPDLY-1 and one CIMS antibody were found only in SLE whereas PTK6, UCHL5 and CIMS (10) were found in RA. Only one disease specific analyte, UBE2C was found in SS. However, a higher number of disease specific analytes (n=10) was found in SV, and included BKT, CIMS (11), CIMS (23), CIMS (16) INADL-1, Sialyl Lewis x, LUM, DPOLM, β-galactosidase and TOP1B. A summary including the top 25 antibodies and their specific targets for each disease, are presented in Supplementary table S3. Out of those top 25 lists, most analytes within SLE, RA and SS were found to be upregulated (15, 21 and 25 respectively), whereas the opposite, e.g. downregulation of most analytes (n=23) was observed in SV. Accordingly, the overlap with the condensed biomarker signatures for respective diseases was also investigated and revealed some overlap. Altogether, these results indicated that biologic events, including deregulation of specific analytes for each disease type, could be identified which may indicate different pathogenetic routes, and which can be used to further understand the complexity behind disease progression and for further diagnostic tools.

### Discussion

Autoimmune diseases today pose a global health issue, affecting millions of people around the globe (29). Diffuse, general symptoms, such as fatigue, inflammation and joint pain, that change in severity over time, shared among several diseases, make clinical diagnosis challenging and there is an urgent need for refined clinical tools for early and differential diagnosis. In this study, candidate biomarker signatures for the autoimmune diseases RA, SLE, SS and SV, were pinpointed. Altogether the results showed that leave-one-out cross validation analysis including all antibodies (n=393) could accurately classify individual AIDs at AUC-values ranging between 0.955-0.803 (FIG. 3). In addition, panels including 40 antibodies could still classify the autoimmune diseases at high accuracy, with AUC values ranging between 0.964-0.793 (FIG. 4). These results show that using a multiplexed approach to reflect the pathogenetic complexity in autoimmune disorders looks very promising and is a venue to continue and explore in order to identify new targets for early and differential diagnosis of autoimmune diseases. The need for better biomarkers in autoimmune diseases is huge. Blood-based biomarkers constitute a simple non-invasive approach, suitable for both discovery biomarker analysis as for the clinical setting and constitute a major ground within the autoimmune community research. Although a few biomarkers have been found as earlier manifestations for disease, such as the presence of antinuclear antibodies (ANAs) in SLE (30, 31) and a-CCP in RA (32, 33), many biomarkers either display too low a specificity and/or sensitivity, are used one-by-one or too few to reflect the complexity in disease (16, 34). Biomarkers for differential diagnosis are thus difficult to identify, and refined tools for correct and early diagnosis are urgently needed to prevent severe organ and tissue related damage. This study utilized an antibody microarray platform targeting mainly immunoregulatory proteins and has an advantage when it comes to identifying levels of proteomic changes within systemic autoimmune disorders, as previously demonstrated by the delivery of candidate biomarkers signatures for classification of SLE and systemic sclerosis, and SLE disease activity (17, 18, 27, 35, 36).

Based from the classification analysis, SLE and SV were found to be easiest to separate from the others (AUC values of 0.955 and 0.937 respectively) while RA and SS were a bit more difficult to separate (AUC=0.858 and AUC=0.803 respectively) (FIGS. 3 and 4). This may partly be explained by the fact that Sjögren syndrome often overlaps in patients with SLE and RA and similar pathogenetic mechanisms have been suggested (3, 4). To our knowledge, samples in this study were collected from patients diagnosed with primary Sjögren syndrome. However, it cannot be ruled out that some RA and SLE patients may have developed SS later, which could contribute to the lower AUC-values. In addition, RA patients are difficult to diagnose since the symptoms of disease often mimic the ones of other inflammatory diseases, especially in early stages of disease. Analysing of the serum proteome in patients with primary but also secondary Sjögren syndrome, RA and SLE would indeed be of great value for decoding underlying molecular pathways, which would be important from a diagnostic and therapeutic perspective.

Important to address, is the low number of samples used in this study which confer a limiting factor since an independent data set for validation was lacking. The use of supervised learning algorithms may pose a problem when its applied in small data sets due to the risk of overfitting, which may lead to poor performance in new sample sets (38, 39). Considering this, the approach used for feature extraction and subsequent generation of condensed signatures in this study was carefully selected to avoid the risk of overtraining. Ultimately, a short signature with high predictive power may always from a logistical and cost-effective view be preferred. However, there is always a trade between the length of the signature and performance, which is why we in this first study, compromised to include 40 antibodies in the final consensus lists. Also, the high number of antibodies most likely reflect that pinpointed diseases do share similar pathogenetic pathways, and thus a higher number of antibodies for differential diagnosis, may from this perspective be necessary. This may also be supported by the major overlap of analytes observed from the differential analysis (Wilcoxon) (FIG. 3 and Supplemental table S2), which further stress the significance of larger data sets to contribute even more stringent analysis.

Based from the differential protein expression analysis, only a small number of disease specific analytes were found (Supplemental table S3B-E, FIG. 5B). The complement system is highly involved in the pathogenesis of autoimmune diseases (37) and the major overlap of deregulated analytes may suggest similar molecular mechanisms underlying disease progression in autoimmunity. Only one analyte, UBEC2, was found uniquely in SS. UBEC2, is a member of the a ubiquitin-conjugating enzyme family which is involved in the process of destruction of mitotic cyclins and for cell cycle progression (38, 39). Interestingly, Ro52 has previously been identified as an E3 ubiquitin ligase of which increased expression may lead to increased apoptosis and for promoting auto reactivity as in the generation of Ro52 autoantibodies (40). Compared to the other AIDs, the majority of analytes were found to be downregulated among SV samples, which could explain the high number of differentially expressed analytes within this group. The reason for this difference however, can only be speculated, but may indicate that the underlying molecular events taking place in systemic vasculitis, is different from the other three diseases. Considering that vasculitis is more common in SLE patients this is unexpected and further studies aiming at these two groups would be of particular interest. Further studies, with bigger sample sets stratified by disease phenotype may help to clarify the underlying role of disease specific analytes and to aid in the search for novel candidate biomarkers for therapeutic strategies.

In this study several analytes, involved in immunoregulatory response, were found to be deregulated among the AIDs compared to the healthy controls (Supplemental table S3A). As expected, one of the upregulated analytes was TNF-α which already has been demonstrated as a promising therapeutic target for treatment with biological TNF inhibitors, especially in RA (41, 42). Other analytes included the pro-inflammatory cytokine IL-6, which also is highly interesting from a therapeutic perspective when it comes to treatment of blockade strategy treatment in autoimmune diseases (43). The level of Osteopontin has previously been demonstrated to be elevated in SLE patients which we could confirm in this study. Osteopontin has been suggested to be associated with SLE development and a potential marker for SLE activity and organ damage (44). Altogether, these data suggest that a more general autoimmune signature may be present, including several already known and novel markers that may play significant roles within autoimmunity. In addition, the finding of a candidate biomarker signature for classification of AIDs from healthy controls, which is supported also from other studies (14) further strengthen the potential of using our antibody microarray platform for biomarker discovery in autoimmune diseases. A tool, able to function as sensor for autoimmune diseases, resulting in the transferal of patients to the right instance, would be of high significance for early and correct diagnosis.

The four systemic autoimmune diseases (SLE, RA, SS and SV) analysed in this study were chosen based on the fact that they share many clinical symptoms and in addition, three of them e.g. SLE, RA and SS, are among the most common AIDs. SV is not that common, though associated with a very poor prognosis.

### Conclusion

We here demonstrate that a general AID biomarker signature could be delineated and that individual AIDs (SLE, RA, SS and SV) could be classified at high accuracies using a multiplexed microarray. These results together with previous studies (15, 16, 27, 34), suggest the fact that the use of a multiplexed approach is more suitable for decoding multifactorial diseases such as autoimmune diseases and will play a significant role for future diagnostic purposes, essential to prevent severe organ and tissue related damage.

### Supplementary Data

### Defining a Condensed Biomarker Signature

A linear support vector machine (SVM) was used as the classification method when defining the condensed biomarker signature. See the scripts detailed in Supplementary table S4.

To rank a given signature a 5-fold cross validation scheme, repeated 15 times, was used as follows: (i) For each training dataset an SVM model was trained. (ii) The corresponding validation dataset was used to estimate the importance of each individual protein in the signature. This was accomplished by removing a given protein (i.e. replacing its expression value by the mean value over all samples) and measure the change of the validation performance. An important protein will result in a large decrease of the validation performance. This procedure was repeated for each validation dataset in the repeated K-fold cross validation procedure. The average change of validation performance for each protein was then computed, giving a final ranking list of all proteins in the signature.

To obtain an unbiased estimate of the performance of a condensed signature, according to the computed ranking list, it is not possible to again use the dataset used to obtain the ranking of the proteins. An additional test set is needed. To this end an outer 5-fold cross validation loop, repeated two times, was introduced with the purpose of evaluating condensed signatures of different lengths. The average test AUC value was used as the estimate of the performance of a condensed signature with a given length.

The different ranking lists generated by the outer loop are slightly different from each other in terms of the rank of a specific protein. The final condensed signature of a given length was assembled by log-rank averaging of all ten lists.

### Supplementary Tables

SUPPLEMENTARY TABLE S1
The number of antibodies targeting the specific proteins. The molecular design of the antibodies display high on-chip
functionality and has been validated in terms of specificity, affinity and performance using MesoScale Discovery, ELISA, MS and SPR-analysis.

| | | No. of | | | No. of | | | No. of |
|---|---|---|---|---|---|---|---|---|
| | | anti- | | | anti- | | | anti- |
| | | body | | | body | | | body |
| Protein | Full name | clones | Protein | Full name | clones | Protein | Full name | clones |
| AKT3 | RAC-gamma | 2 | IgM | IgM | 5 | OSTP | Osteopontin | 3 |
| | serine/threoninE−protein | | | | | | | |
| | kinase | | | | | | | |
| Angiomotin | Angiomotin | 2 | IL-10 | Interleukin-10 | 3 | OTU6B | OTU domain-containing | 2 |
| | | | | | | | protein 6B | |
| ANM5 | Protein arginine N- | 2 | IL-11 | Interleukin-11 | 3 | OTUB1-1 | Ubiquitin thioesterase | 2 |
| | methyltransferase 5 | | | | | | OTUB1 | |
| APLF | Aprataxin and PNK-like | 2 | IL-12 | Interleukin-12 | 4 | OTUB2-1 | Ubiquitin thioesterase | 2 |
| | factor | | | | | | OTUB2 | |
| APOA4 | Apolipoprotein A4 | 3 | IL-13 | Interleukin-13 | 3 | P85A | Phosphatidylinositol 3- | 3 |
| | | | | | | | kinase regulatory subunit | |
| | | | | | | | alpha | |
| Apolipoprotein | Apolipoprotein A1 | 3 | IL-16 | Interleukin-16 | 3 | PAK4-1 | Serine/threoninE−protein | 2 |
| A1 | | | | | | | kinase PAK 4 | |
| ARHGC-1 | Rho guanine nucleotide | 1 | IL-18 | Interleukin-18 | 3 | PAK5 | Serine/threoninE−protein | 2 |
| | exchange factor 12 | | | | | | kinase PAK 7 | |
| ATP5B | ATP synthase subunit | 3 | IL-b-ra | Interleukin-1 receptor | 3 | PARP1 | Poly [ADP-ribose] | 1 |
| | beta, mitochondria! | | | antagonist protein | | | polymerase 1 | |
| BIRC2 | Baculoviral IAP repeat- | 2 | IL-1α | Interleukin-1 alpha | 3 | PARP6B | Partitioning defective 6 | 2 |
| | containing protein 2 | | | | | | homolog beta | |
| BKT | TyrosinE−protein kinase | 4 | IL-1β | Interleukin-1 beta | 3 | PGAM5 | Serine/threoninE−protein | 2 |
| | BTK | | | | | | phosphatase PGAM5, | |
| | | | | | | | mitochondrial | |
| C1 | Plasma protease C1 | 4 | IL-2 | Interleukin-2 | 3 | PRD14 | PR domain zinc finger | 2 |
| esterase | inhibitor | | | | | | protein 14 | |
| inhibitor | | | | | | | | |
| C1q | Complement C1q | 1 | IL-3 | Interleukin-3 | 3 | PRDM8-1 | PR domain zinc finger | 2 |
| | | | | | | | protein 8 | |
| C1s | Complement C1s | 1 | IL-4 | Interleukin-4 | 4 | PRKCZ | Protein kinase C zeta type | 2 |
| C3 | Complement C3 | 6 | IL-5 | Interleukin-5 | 3 | PRKG2 | cGMP-dependent protein | 2 |
| | | | | | | | kinase 2 | |
| C4 | Complement C4 | 4 | IL-6 | Interleukin-6 | 8 | Procathepsin | Cathepsin W | 1 |
| | | | | | | W | | |
| C5 | Complement C5 | 3 | IL-7 | Interleukin-7 | 2 | Properdin | Properdin | 1 |
| | | | | | | (Factor P) | | |
| CBPP22 | Calcineurin B | 2 | IL-8 | Interleukin-8 | 3 | PSA | ProstatE−specific antigen | 1 |
| | homologous protein 1 | | | | | | | |
| CD40 | CD40 protein | 4 | IL-9 | Interleukin-9 | 3 | PTK6 | Protein-tyrosine kinase 6 | 1 |
| CD40L | CD40 ligand | 1 | INADL- | InaD-like protein | 2 | PTN13-1 | TyrosinE−protein | 2 |
| | | | 1 | | | | phosphatase non-receptor | |
| | | | | | | | type 13 | |
| CDK2 | Cyclin-dependent kinase | 2 | Integrin | Integrin alpha-10 | 1 | PTPN1 | TyrosinE−protein | 3 |
| | 2 | | α-10 | | | | phosphatase non-receptor | |
| | | | | | | | type 1 | |
| CENTG1 | Arf-GAP with GTPase, | 2 | Integrin | Integrin alpha-11 | 1 | PTPRD | Receptor-type tyrosinE− | 2 |
| | ANK repeat and PH | | α-11 | | | | protein phosphatase delta | |
| | domain-containing | | | | | | | |
| | protein 2 | | | | | | | |
| CHEK2 | Serine/threoninE−protein | 2 | ITCH-2 | E3 ubiquitin-protein | 2 | PTPRJ | Receptor-type tyrosinE− | 2 |
| | kinase Chk2 | | | ligase Itchy homolog | | | protein phosphatase eta | |
| CHX10 | Visual system homeobox | 3 | JAK3 | TyrosinE−protein | 1 | PTPRK | Receptor-type tyrosinE− | 2 |
| | 2 | | | kinase JAK3 | | | protein phosphatase kappa | |
| CSNK1E | Casein kinase 1 isoform | 2 | KCC2B-3 | Calcium/calmodulin- | 2 | PTPRN2 | Receptor-type tyrosinE− | 2 |
| | epsilon | | | dependent protein | | | protein phosphatase N2 | |
| | | | | kinase type 11 subunit | | | | |
| | | | | beta | | | | |
| CT17 | Choleratoxin subunit B | 1 | KCC4 | Calcium/calmodulin- | 2 | PTPRO | Receptor-type tyrosinE− | 2 |
| | | | | dependent protein | | | protein phosphatase O | |
| | | | | kinase type IV | | | | |
| Cystatin C | Cystatin-C | 4 | Keratin | Keratin, type I | 3 | PTPRT | Receptor-type tyrosinE− | 2 |
| | | | 19 | cytoskeletal 19 | | | protein phosphatase T | |
| DCNL1 | DCN1-like protein 1 | 2 | KIAA0882 | TBC1 domain family | 3 | RANTES | C-C motif chemokine 5 | 3 |
| | | | | member 9 | | | | |
| | | | | Calcium/calmodulin- | 2 | RPS6KA2 | Ribosomal protein S6 | 3 |
| Digoxin | Digoxin | 1 | KKCC1-1 | dependent protein | | | kinase alpha-2 | |
| | | | | kinase kinase 1 | | | | |
| DLG1-1 | Disks large homolog 1 | 2 | KRASE | GTPase KRas | 1 | SHC1 | SHC-transforming protein 1 | 2 |
| DLG2-1 | Disks large homolog 2 | 2 | KSYK | TyrosinE−protein | 2 | Sialyl Lewis x | Sialyl Lewis x | 1 |
| | | | | kinase SYK | | | | |
| DLG4-2 | Disks large homolog 4 | 2 | LDL | Apolipoprotein B-100 | 2 | SNTA1 | Alpha-1-syntrophin | 2 |
| DPOLM | DNA-directed DNA/RNA | 2 | Leptin | Leptin | 1 | Sox11a | Transcription factor SOX- | 1 |
| | polymerase mu | | | | | | 11 | |
| DUSP7 | Dual specificity protein | 2 | Lewis x | Lewis x | 2 | SPDLY-1 | Protein Spindly | 2 |
| | phosphatase 7 | | | | | | | |
| DUSP9 | Dual specificity protein | 1 | Lewis y | Lewis y | 1 | STAP1 | Signal-transducing adaptor | 2 |
| | phosphatase 9 | | | | | | protein 1 | |
| EGFR | Epidermal growth factor | 1 | LIN7A | Protein lin-7 homolog | 2 | STAP2 | Signal-transducing adaptor | 4 |
| | receptor | | | A | | | protein 2 | |
| Eotaxin | Eotaxin | 3 | LUM | Lumican | 1 | STAT1 | Signal transducer and | 2 |
| | | | | | | | activator of transcription 1- | |
| | | | | | | | alpha/beta | |
| Factor B | Complement factor B | 4 | MAGI1- | MembranE−associated | 2 | TENS4 | Tensin-4 | 1 |
| | | | 1 | guanylate kinase, WW | | | | |
| | | | | and PDZ domain- | | | | |
| | | | | containing protein 1 | | | | |
| FASN | FASN protein | 4 | MAP2K | Dual specificity | 2 | TGF-β | Transforming growth factor | 3 |
| | | | 2 | mitogen-activated | | | beta-1 | |
| | | | | protein kinase kinase 2 | | | | |
| FER | TyrosinE−protein kinase | 2 | MAP2K | Dual specificity | 2 | TM peptide | TM peptide | 1 |
| | Fer | | 6 | mitogen-activated | | | | |
| | | | | protein kinase kinase 6 | | | | |
| GAK | GAK protein | 3 | MAPK9 | Mitogen-activated | 2 | TNFRSF14 | Tumor necrosis factor | 2 |
| | | | | protein kinase 9 | | | receptor superfamily | |
| | | | | | | | member 14 | |
| GEM | GTP-binding protein | 2 | MARK1-1 | Serine/threoninE− | 2 | TNFRSF3 | Tumor necrosis factor | 3 |
| | GEM | | | protein kinase MARK1 | | | receptor superfamily | |
| | | | | | | | member | |
| GLP-1 | Glucagon-like peptidE−1 | 1 | MARK2-1 | Serine/threoninE− | 2 | TNF-α | Tumor necrosis factor | 3 |
| | | | | protein kinase MARK2 | | | | |
| GLP-1R | Glucagon-like peptide 1 | 1 | MATK | MegakaryocytE− | 3 | TNF-β | Lymphotoxin-alpha | 4 |
| | receptor | | | associated tyrosinE− | | | | |
| | | | | protein kinase | | | | |
| GM-CSF | GranulocytE−macrophage | 6 | MCP-1 | C-C motif chemokine 2 | 9 | TOPB1 | DNA topoisomerase 2- | 2 |
| | colony-stimulating factor | | | | | | binding protein 1 | |
| GNAI3 | Guanine nucleotidE− | 2 | MCP-3 | C-C motif chemokine 7 | 3 | UBC9 | SUMO-conjugating enzyme | 3 |
| | binding protein G(k) | | | | | | UBC9 | |
| | subunit alpha | | | | | | | |
| GORS2-1 | Golgi reassembly- | 2 | MCP-4 | C-C motif chemokine | 3 | UBE2C | Ubiquitin-conjugating | 2 |
| | stacking protein 2 | | | 13 | | | enzyme E2 C | |
| GPRK5 | G protein-coupled | 1 | MD2L1 | Mitotic spindle | 2 | UBP7 | Ubiquitin carboxyl-terminal | 2 |
| | receptor kinase 5 | | | assembly checkpoint | | | hydrolase 7 | |
| | | | | protein MAD2A | | | | |
| GRIP2-1 | Glutamate receptor- | 2 | MK01 | Mitogen-activated | 4 | UCHL5 | Ubiquitin carboxyl-terminal | 1 |
| | interacting protein 2 | | | protein kinase 1 | | | hydrolase isozyme L5 | |
| HADH2 | HADH2 protein | 4 | MK08 | Mitogen-activated | 3 | UPF3B | Regulator of nonsense | 2 |
| | | | | protein kinase 8 | | | transcripts 3B | |
| Her2/ErbB2 | Receptor tyrosinE−protein | 4 | Mucin-1 | Mucin-1 | 6 | VEGF | Vascular endothelial growth | 4 |
| | kinase erbB-2 | | | | | | factor | |
| HLA- | HLA-DR/DP | 1 | MYOM | Myomesin-2 | 2 | β- | | |
| DR/DP | | | 2 | | | galactosidase | Beta-galactosidase | 1 |
| ICAM-1 | Intercellular adhesion | 1 | NDC80 | Kinetochore protein | 2 | | | |
| | molecule 1 | | | NDC80 homolog | | | | |
| IFN-γ | Interferon gamma | 3 | NOS1-1 | Nitric oxide synthase, | 2 | | | |
| | | | | brain | | | | |
| | | | OSBPL | Oxysterol-binding | 2 | | | |
| | | | 3 | protein-related protein | | | | |
| | | | | 3 | | | | |

SUPPLEMENTARY TABLE S2
Condensed panels of antibodies, based on a ranking procedure combined with a K-fold cross validation. The individual
scFv antibody clone number is shown in brackets 0.

| (A) H vs. | | (B) SLE vs. | | | | | | | |
|---|---|---|---|---|---|---|---|---|---|
| SLE + RA + SS + SV | | RA + SS + SV | | (C) RA vs. SLE + SS + SV | | (D) SS vs. SLE + RA + SV | | (E) SV vs. SLE + RA + SS | |
| Antibody | Rank | Antibody | Rank | Antibody | Rank | Antibody | Rank | Antibody | Rank |
| C3 (4) | 1.0 | C3 (4) | 1.2 | C3 (4) | 1.7 | C1 inh. (2) | 1.5 | Factor B (3) | 1.2 |
| C3 (6) | 2.5 | C3 (6) | 1.7 | VEGF (3) | 4.3 | C1 inh. (4) | 2.1 | Apo-A1 (3) | 2.5 |
| IL-4 (3) | 3.4 | C3 (5) | 3.5 | KKCC1-1 (1) | 4.7 | OTUB1-1 (2) | 2.8 | C3 (6) | 3.0 |
| C3 (3) | 4.2 | C4 (2) | 3.5 | CIMS (10) | 5.5 | C1 inh. (3) | 5.5 | C3 (3) | 5.8 |
| C5 (3) | 5.3 | C3 (3) | 5.8 | CIMS (4) | 5.8 | MCP-1 (3) | 6.4 | CIMS (27) | 6.8 |
| C3 (5) | 6.7 | MAGI1-1 (1) | 6.1 | Factor B (2) | 6.9 | HADH2 (3)/GSN | 8.1 | IgM (1) | 9.2 |
| C1 inh. (1) | 8.9 | VEGF (3) | 6.8 | CIMS (13) | 7.8 | Angiomotin (2) | 11.5 | Apo-A1 (2) | 10.0 |
| C4 (4) | 11.7 | C4 (3) | 10.0 | C3 (5) | 7.9 | IL-13 (2) | 12.8 | C4 (4) | 10.9 |
| UBC9 (3) | 14.3 | Apo-A1 (3) | 10.7 | PRKG2 (2) | 9.8 | PRKG2 (2) | 13.2 | KKCC1-1 (1) | 11.1 |
| IL-1ra (3) | 16.4 | C3 (2) | 12.1 | C1 inh. (1) | 12.7 | Factor B (3) | 14.7 | IL-4 (3) | 11.3 |
| CIMS (10) | 17.5 | C1 inh. (1) | 12.6 | GM-CSF (1) | 19.7 | CIMS (25) | 17.8 | C1 inh. (2) | 14.3 |
| CIMS (27) | 17.7 | Apo-A1 (2) | 14.5 | PKB gamma (1) | 19.7 | CSNK1E (2) | 18.2 | C1 inh. (4) | 14.4 |
| GM-CSF (1) | 18.6 | OTUB1-1 (2) | 14.6 | C5 (2) | 20.9 | Apo-A1 (2) | 20.4 | C1q | 15.3 |
| C1 inh. (4) | 18.9 | C5 (3) | 16.7 | MCP-4 (2) | 22.6 | IL-6 (8) | 22.2 | C1 inh. (3) | 17.5 |
| IL-1a (1) | 21.3 | IL-6 (6) | 17.1 | LUM | 22.9 | IL-3 (3) | 24.2 | C4 (3) | 17.9 |
| TNF-a (2) | 24.8 | IL-4 (3) | 21.0 | PTN13-1 (1) | 24.2 | CIMS (8) | 26.4 | CIMS (10) | 19.0 |
| HADH (3)/GSN | 26.3 | PAK7 (1) | 24.3 | Factor B (3) | 24.4 | PTPRD (1) | 28.5 | IgM (2) | 20.5 |
| MAGI1-1 (1) | 26.6 | TNF-a (1) | 28.5 | C3 (6) | 26.2 | IL-6 (5) | 28.9 | Angiomotin (2) | 23.8 |
| KCC2B-3 (1) | 28.8 | Lewis x (1) | 29.5 | IgM (1) | 26.7 | ARHGC-1 (1) | 36.4 | PTPRD (1) | 24.1 |
| LUM | 31.0 | GM-CSF (6) | 30.1 | EGFR | 27.6 | Properdin | 37.1 | MCP-1 (9) | 25.9 |
| UPF3B (2) | 31.6 | C4 (4) | 32.4 | NOS1-1 (2) | 29.5 | CIMS (28) | 37.7 | C4 (2) | 27.9 |
| GLP-1 R | 33.7 | NOS1-1 (2) | 33.2 | Procathepsin W | 30.8 | DCNL1 (2) | 39.1 | IL-3 (3) | 30.4 |
| FASN (4) | 34.9 | DPOLM (2) | 33.5 | IgM (5) | 31.0 | PRKG2 (1) | 39.7 | CIMS (14) | 32.1 |
| TNF-a (1) | 36.1 | TNFRSF3 (2) | 34.9 | Apo-A4 (2) | 32.1 | GAK (3) | 40.1 | GM-CSF (1) | 32.4 |
| TopBP1 (1) | 37.6 | VEGF (2) | 34.9 | IL-9 (1) | 32.7 | TBC1D9 (2) | 41.6 | IL-12 (3) | 32.8 |
| IL-8 (1) | 37.9 | IL-1ra (3) | 35.3 | PTK6 | 33.1 | TNFRSF14 (2) | 42.1 | C5 (3) | 33.2 |
| Factor B (3) | 38.3 | OTUB2-1 (2) | 41.8 | hSpindly (2) | 33.6 | CIMS (9) | 42.1 | Cystatin C (2) | 34.8 |
| FER (1) | 39.0 | IL-13 (2) | 42.3 | GRIP-2 (1) | 36.5 | Factor B (2) | 43.1 | IL-18 (2) | 35.2 |
| CDK-2 (1) | 39.4 | MCP-1 (5) | 46.8 | CHEK2 (1) | 36.6 | MARK2-1 (2) | 43.3 | MUC1 (1) | 38.4 |
| CIMS (16) | 39.5 | MATK (3) | 47.6 | MAPKK 2 (2) | 39.2 | Sox11a | 43.9 | DLG4-2 (2) | 41.6 |
| RPS6KA2 (3) | 39.6 | MUC1 (4) | 47.9 | ITCH-2 (1) | 39.5 | KKCC1-1 (2) | 44.3 | IL-3 (2) | 41.7 |
| GRK5 (1) | 41.2 | IgM (3) | 48.3 | CIMS (8) | 39.5 | MUC1 (5) | 44.7 | CIMS (13) | 42.2 |
| MAPKK 6 (1) | 41.5 | PTPPRN2 (1) | 53.6 | C3 (2) | 40.6 | GEM (2) | 49.1 | UPF3B (1) | 42.7 |
| C4(3) | 42.6 | OTU6B (1) | 54.0 | Cystatin C (1) | 41.4 | MARK1-1 (1) | 49.1 | CT17 | 43.2 |
| UBP7 (1) | 43.3 | MARK2-1 (2) | 55.0 | IgM (2) | 42.2 | C5 (3) | 49.3 | INFRSF14 (2) | 43.7 |
| C4 (1) | 43.4 | BIRC2 (1) | 55.4 | CIMS (9) | 42.8 | RANTES (2) | 49.7 | MCP-1 (8) | 47.3 |
| AGAP-2 (1) | 43.5 | hSpindly (1) | 56.0 | PRKCZ (1) | 48.9 | IL-12 (3) | 51.7 | MAPKK 6 (2) | 48.7 |
| VEGF (3) | 45.0 | IL-4 (4) | 57.1 | IL-1ra (3) | 49.9 | MAPKK 6 (1) | 51.8 | CIMS (25) | 51.8 |
| HLA-DR/DP | 46.9 | HADH2 (4) | 59.9 | IL-4 (1) | 50.5 | IL-8 (2) | 51.8 | TNFRSF3 (3) | 51.9 |
| CIMS (4) | 47.4 | MAPKK 6 (1) | 61.5 | Cystatin C (3) | 51.9 | UPF3B (1) | 52.9 | Angiomotin (1) | 52.4 |

SUPPLEMENTARY TABLE S3
Top 25 differentially expressed analytes from Wilcoxon signed-rank test (q < 0.05), including AUC and 95% Cl. For
analytes targeted by multiple clones, individual clone suffix is shown in brackets.

| | | | | | | | | | (C) RA vs. SLE + SS + SV | | |
|---|---|---|---|---|---|---|---|---|---|---|---|
| | (A) H vs. SLE + RA + SS + SV | | | | (B) SLE vs. Ra + SS + SV | | | | | | 95% |
| Antigen | q-value | AUC | 95% Cl | Antigen | q-value | AUC | 95% Cl | Antigen | q-value | AUC | Cl |
| C3 (4) | 6.11312E−23 | 0.896 | 0.869-0.923 | C4 (2) | 1.92829E−13 | 0.911 | 0.876-0.945 | Cl inh. | 3.74741E−05 | 0.755 | 0.691- |
| | | | | | | | | (1) | | | 0.816 |
| UBC9 (3) | 2.46399E−14 | 0.815 | 0.772-0.857 | C3 (6) | 2.26317E−13 | 0.906 | 0.867-0.941 | PRKG2 | 0.000270946 | 0.733 | 0.662- |
| | | | | | | | | (2) | | | 0.800 |
| C3 (6) | 4.78988E−13 | 0.798 | 0.756-0.838 | C3 (4) | 1.3471E−11 | 0.878 | 0.823-0.926 | KKCC1-1 | 0.000415853 | 0.721 | 0.651- |
| | | | | | | | | (1) | | | 0.791 |
| C3 (5) | 7.46857E−13 | 0.794 | 0.750-0.838 | C3 (3) | 1.72755E−11 | 0.873 | 0.821-0.921 | PTN13-1 | 0.000415853 | 0.722 | 0.649- |
| | | | | | | | | (1) | | | 0.789 |
| C3 (2) | 3.67974E−11 | 0.774 | 0.727-0.820 | C4 (3) | 5.26683E−11 | 0.864 | 0.815-0.910 | IgM (1) | 0.00204718 | 0.698 | 0.629- |
| | | | | | | | | | | | 0.760 |
| RPS6KA2 | 5.19795E−11 | 0.773 | 0.722-0.821 | C4 (4) | 7.43386E−09 | 0.826 | 0.770-0.881 | IgM (5) | 0.00204718 | 0.700 | 0.632- |
| (1) | | | | | | | | | | | 0.770 |
| C3 (3) | 1.19753E−10 | 0.766 | 0.721-0.812 | Apo-A1 | 2.89798E−06 | 0.778 | 0.714-0.834 | PKB | 0.00204718 | 0.694 | 0.615- |
| | | | | (2) | | | | gamma | | | 0.764 |
| | | | | | | | | (1) | | | |
| CIMS (10) | 9.17488E−10 | 0.755 | 0.707-0.801 | VEGF | 1.73961E−05 | 0.758 | 0.695-0.821 | CDK-2 | 0.00204718 | 0.697 | 0.629- |
| | | | | (3) | | | | (2) | | | 0.763 |
| STAP1 (1) | 1.82011E−09 | 0.749 | 0.696-0.799 | Apo-A1 | 1.91513E−05 | 0.754 | 0.691-0.811 | MAPKK | 0.00204718 | 0.695 | 0.622- |
| | | | | (3) | | | | 2 (2) | | | 0.765 |
| P85A (3) | 2.55717E−08 | 0.735 | 0.686-0.783 | MAGI1-1 | 1.96854E−05 | 0.755 | 0.685-0.820 | GLP-1 | 0.00262381 | 0.691 | 0.613- |
| | | | | (1) | | | | | | | 0.757 |
| KCC2B-3 | 3.02553E−08 | 0.732 | 0.684-0.778 | C3 (5) | 0.000042344 | 0.747 | 0.677-0.816 | IgM (2) | 0.00262381 | 0.691 | 0.621- |
| (2) | | | | | | | | | | | 0.760 |
| INADL-1 | 4.1601E−08 | 0.73 | 0.678-0.782 | C1 inh. | 9.27677E−05 | 0.738 | 0.683-0.789 | OTUB2-1 | 0.00271334 | 0.689 | 0.618 - |
| (2) | | | | (3) | | | | (I) | | | 0.759 |
| DLG4-2 (I) | 4.75923E−08 | 0.728 | 0.680-0.773 | IgM (3) | 0.000367382 | 0.722 | 0.656-0.791 | C5 (3) | 0.00343315 | 0.682 | 0.616- |
| | | | | | | | | | | | 0.749 |
| INADL-1 | 9.039E−08 | 0.726 | 0.670-0.781 | NOS1-1 | 0.000392197 | 0.721 | 0.651-0.794 | IL-6 (7) | 0.00343315 | 0.687 | 0.613- |
| (1) | | | | (1) | | | | | | | 0.757 |
| Osteopontin | 1.71762E−07 | 0.72 | 0.660-0.772 | C1 inh. | 0.000592225 | 0.715 | 0.657-0.774 | SNTA1 | 0.00343315 | 0.683 | 0.611- |
| (2) | | | | (4) | | | | (1) | | | 0.763 |
| UCHL5 | 2.22831E−07 | 0.719 | 0.664-0.769 | BIRC2 | 0.000898398 | 0.711 | 0.644-0.779 | CHEK2 | 0.00343315 | 0.684 | 0.613- |
| | | | | (2) | | | | (1) | | | 0.749 |
| C4 (2) | 2.65945E−07 | 0.717 | 0.669-0.761 | C1 inh. | 0.000944125 | 0.709 | 0.650-0.763 | MATK | 0.00392043 | 0.680 | 0.606- |
| | | | | (2) | | | | (1) | | | 0.751 |
| MARK1-1 | 3.98633E−07 | 0.712 | 0.657-0.763 | IgM (2)* | 0.00137751 | 0.703 | 0.641-0.763 | IL-1b | 0.00473801 | 0.677 | 0.606- |
| (2) | | | | | | | | (2) | | | 0.750 |
| C5 (3) | 4.38712E−07 | 0.712 | 0.661-0.761 | GM-CSF | 0.00148405 | 0.700 | 0.627-0.773 | TNFRSF3 | 0.00473801 | 0.677 | 0.605- |
| | | | | (1) | | | | (2) | | | 0.743 |
| PRKCZ (1) | 1.8135E−06 | 0.702 | 0.643-0.758 | GAK (2) | 0.00156823 | 0.701 | 0.630-0.772 | DLG4-2 | 0.00473801 | 0.677 | 0.605- |
| | | | | | | | | (2) | | | 0.746 |
| C3 (1) | 2.27779E−06 | 0.7 | 0.646-0.752 | INFRSF14 | 0.00178729 | 0.697 | 0.626-0.766 | IL-8 (2) | 0.00549152 | 0.673 | 0.598- |
| | | | | (1) | | | | | | | 0.745 |
| STAP1 (2) | 2.29734E−06 | 0.702 | 0.650-0.752 | Lewis y | 0.00205928 | 0.695 | 0.620-0.767 | TNF-b | 0.00559346 | 0.672 | 0.599- |
| | | | | | | | | (3) | | | 0.742 |
| KKCC1-1 | 2.55033E−06 | 0.698 | 0.649-0.747 | IL-4 (3) | 0.0021822 | 0.691 | 0.621-0.761 | Lewis y | 0.00578037 | 0.669 | 0.592- |
| (2) | | | | | | | | | | | 0.746 |
| PTPN1 (3) | 3.68037E−06 | 0.697 | 0.642-0.750 | MCP-1 (9) | 0.0021822 | 0.696 | 0.619-0.767 | NOS1-1 | 0.00578037 | 0.672 | 0.598- |
| | | | | | | | | (2) | | | 0.740 |
| hSpindly | 3.68037E−06 | 0.695 | 0.642-0.751 | Cystatin | 0.00251973 | 0.690 | 0.620-0.758 | IL-lb | 0.00619337 | 0.670 | 0.599- |
| (2) | | | | C (2) | | | | (1) | | | 0.743 |

| | (D) SS vs. SLE + RA + SV | | | | (E) SV vs. SLE + RA + SS | | |
|---|---|---|---|---|---|---|---|
| Antigen | q-value | AUC | 95% Cl | Antigen | q-value | AUC | 95% Cl |
| MATK (2) | 1.95164E−05 | 0.718 | 0.658-0.775 | IgM (2) | 1.17114E−15 | 0.844 | 0.797-0.884 |
| GEM (2) | 1.95164E−05 | 0.723 | 0.665-0.781 | IgM (1) | 1.42329E−13 | 0.817 | 0.772-0.863 |
| Her2/ErbB2 | 2.39878E−05 | 0.705 | 0.644-0.767 | IL-4 (3) | 2.45402E−13 | 0.812 | 0.760-0.860 |
| (2) | | | | | | | |
| MATK (3) | 2.39878E−05 | 0.705 | 0.644-0.762 | Angiomotin | 4.55055E−13 | 0.810 | 0.760-0.856 |
| | | | | (2) | | | |
| GEM (1) | 2.39878E−05 | 0.710 | 0.653-0.764 | GM-CSF | 1.05443E−12 | 0.804 | 0.755-0.851 |
| | | | | (1) | | | |
| PAR-6B (2) | 2.39878E−05 | 0.707 | 0.644-0.766 | MUC1 (1) | 1.53178E−12 | 0.802 | 0.753-0.848 |
| DCNL1 (2) | 2.39878E−05 | 0.705 | 0.646-0.758 | IL-11 (2) | 1.70405E−12 | 0.801 | 0.750-0.848 |
| ITCH-2 (1) | 2.39878E−05 | 0.709 | 0.648-0.771 | PSA | 1.71093E−12 | 0.799 | 0.747-0.849 |
| FER (1) | 2.39878E−05 | 0.710 | 0.649-0.772 | RANTES | 3.34687E−12 | 0.794 | 0.742-0.845 |
| | | | | (1) | | | |
| KSYK (1) | 2.84351E−05 | 0.703 | 0.646-0.760 | IL-12 (3) | 3.34687E−12 | 0.795 | 0.742-0.842 |
| HADH2 | 5.49172E−05 | 0.694 | 0.635-0.752 | IL-4 (2) | 6.81591E−12 | 0.792 | 0.742-0.841 |
| (3)/GSN | | | | | | | |
| CSNK1E | 5.49172E−05 | 0.695 | 0.634-0.759 | UPF3B (1) | 7.46307E−12 | 0.790 | 0.739-0.837 |
| (2) | | | | | | | |
| GORS2-1 | 6.66891E−05 | 0.695 | 0.630-0.755 | IL-3 (2) | 7.63932E−12 | 0.788 | 0.736-0.837 |
| (2) | | | | | | | |
| IL-3 (3) | 7.03667E−05 | 0.692 | 0.634-0.749 | CT17 | 1.04423E−11 | 0.787 | 0.731-0.838 |
| Her2/ErbB2 | 7.03667E−05 | 0.690 | 0.627-0.750 | PTPRD (1) | 1.04423E−11 | 0.787 | 0.740-0.834 |
| (1) | | | | | | | |
| TNFRSF14 | 7.03667E−05 | 0.692 | 0.629-0.759 | BTK (1) | 1.31095E−11 | 0.784 | 0.730-0.837 |
| (2) | | | | | | | |
| UBP7 (1) | 9.32392E−05 | 0.688 | 0.620-0.750 | TNF-b (1) | 1.67381E−11 | 0.782 | 0.732-0.830 |
| R-PTP-eta | 9.32392E−05 | 0.687 | 0.625-0.749 | IgM (3) | 2.02089E−11 | 0.782 | 0.731-0.833 |
| (2) | | | | | | | |
| DCNL1 (1) | 9.32392E−05 | 0.687 | 0.627-0.747 | IL-10 (3) | 2.02813E−11 | 0.782 | 0.725-0.835 |
| LIN7A (2) | 9.32392E−05 | 0.687 | 0.621-0.750 | MCP-4 (1) | 3.10126E−11 | 0.778 | 0.723-0.830 |
| RANTES | 0.000106877 | 0.684 | 0.622-0.744 | CD40 | 3.10126E−11 | 0.780 | 0.726-0.829 |
| (2) | | | | ligand | | | |
| KCC2B-3 | 0.000113498 | 0.684 | 0.620-0.746 | MCP-4 (3) | 3.10126E−11 | 0.778 | 0.722-0.832 |
| (1) | | | | | | | |
| Her2/ErbB2 | 0.000132889 | 0.683 | 0.615-0.740 | TBC1D9 | 3.10126E−11 | 0.779 | 0.723-0.834 |
| (3) | | | | (2) | | | |
| CHP1 (1) | 0.000142836 | 0.683 | 0.620-0.745 | PAR-6B | 3.10126E−11 | 0.777 | 0.721-0.829 |
| | | | | (1) | | | |
| IL-lb (3) | 0.000145011 | 0.681 | 0.620-0.743 | C4 (3) | 3.38326E−11 | 0.775 | 0.726-0.822 |

SUPPLEMENTARY TABLE S5
Amino acid sequences for CIMS antibodies used in the
Examples

| Clone index | | |
|---|---|---|
| motif | Peptide sequence | Amino acid sequence |
| CIMS (4) | GIVKYLYEDEG | EVQLLESGGGLVQPGGSLRLSCAASGFTFSNYAMHWVRQ |
| | | APGKGLEWVSSISNRGSRTFYADSVKGRFTISRDNSKNTLYL |
| | | QMNSLRAEDTAVYYCARDHRWDPGAFDIWGQGTLVTVSS |
| | | GGGGSGGGGSGGGGSQSVLTQPPSASGTPGQRVTISCTGS |
| | | SSNIGADYDVHWYQQLPGTAPKLLIYGNSNRPSGVPDRFS |
| | | GSKSGTSASLAISGLRSEDEADYYCAAWDDGLSGVVFGGGT |
| | | KLTVLGEQKLISEEDLSGSAAAHHHHHH |
| CIMS (8) | DFAEDK | EVQLLESGGGLVQPGGSLRLSCAASGFTFSSYAMSWVRQA |
| | | PGKGLEWVSSISSSSSYIYYADSVKGRFTISRDNSKNTLYLQM |
| | | NSLRAEDTAVYYCARLFFSGGATRAAFDIWGQGTLVTVSSG |
| | | GGGSGGGGSGGGGSQSVLTQPPSASGTPGQRVTISCSGGS |
| | | SNIGSNTVNWYQQLPGTAPKLLIYGNSNRPSGVPDRFSGSK |
| | | SGTSASLAISGLRSEDEADYYCAAWDDSLNGRVFGGGTKLT |
| | | VLGEQKLISEEDLSGSAAAHHHHHH |
| CIMS (9) | LTEFAK | EVQLLESGGGLVQPGGSLRLSCAASGFTFSDYYMSWIRQAP |
| | | GKGLEWVSSISSRSSYIYYADSVKGRFTISRDNSKNTLYLQM |
| | | NSLRAEDTAVYYCAKDREYYDILTGYPSMDVWGQGTLVTV |
| | | SSGGGGSGGGGSGGGGSQSVLTQPPSASGTPGQRVTISCT |
| | | GSSSNIGAGYDVHWYQQLPGTAPKLLIYDNNKRPSGVPDR |
| | | FSGSKSGTSASLAISGLRSEDEADYYCSAWDESLSGVVFGGG |
| | | TKLTVLGEQKLISEEDLSGSAAAHHHHHH |
| CIMS (10) | TEEQLK | EVQLLESGGGLVQPGGSLRLSCAASGFTFSSYGMHWVRQA |
| | | PGKGLEWVSAISGSGGSTYYADSVKGRFTISRDNSKNTLYLQ |
| | | MNSLRAEDTAVYYCARSRYGSGMDVWGQGTLVTVSSGG |
| | | GGSGGGGSGGGGSQSVLTQPPSASGTPGQRVTISCSGSSS |
| | | NVGVNYVYWYQQLPGTAPKLLIYSHNQRPSGVPDRFSGSK |
| | | SGTSASLAISGLRSEDEADYYCAAWDDSLNGVVFGGGTKLT |
| | | VLGEQKLISEEDLSGSAAAHHHHHH |
| CIMS (13) | SSAYSR | EVQLLESGGGLVQPGGSLRLSCAASGFTFSSYAMSWVRQA |
| | | PGKGLEWVSAISGSGGRTYYTDSVRDRFTISRDNSKNTLYLQ |
| | | MNSLRAEDTAVYYCARDLMPVCQYCYGMDVWGQGTLVT |
| | | VSSGGGGSGGGGSGGGGSQSVLTQPPSASGTPGQRVTISC |
| | | TGSSSNIGAGYDVHWYQQLPGTAPKLLIYSNNQRPSGVPD |
| | | RFSGSKSGTSASLAISGLRSEDEADYYCQSYDSSLNKDVVFG |
| | | GGTKLTVLGEQKLISEEDLSGSAAAHHHHHH |
| CIMS (14) | SSAYSR | EVQLLESGGGLVQPGGSLRLSCAASGFTFSDYYMSWVRQA |
| | | PGKGLEWVADIKRDGSTRYYGDSVKGRFTISRDNSKNTLYL |
| | | QMNSLRAEDTAVYYCARDRLVAGLFDYWGQGTLVTVSSG |
| | | GGGSGGGGSGGGGSQSVLTQPPSASGTPGQRVTISCTGSS |
| | | SNIGAGYDVHWYQQLPGTAPKLLIYGNSNRPSGVPDRFSG |
| | | SKSGTSASLAISGLRSEDEADYYCAAWDDSLSVLFGGGTKLT |
| | | VLGEQKLISEEDLSGSAAAHHHHHH |
| CIMS (16) | EDFR | EVQLLESGGGLVQPGGSLRLSCAASGFTFDDYGMSWVRQ |
| | | APGKGLEWVSAISGSGGSTYYADPVKGRFTISRDNSKNTLYL |
| | | QMNSLRAEDTAVYYCARSRYGSGMDVWGQGTLVTVSSG |
| | | GGGSGGGGSGGGGSQSVLTQPPSASGTPGQRVTISCSGSS |
| | | SNIGSNYVYWYQQLPGTAPKLLIYKSNQRPSGVPDRFSGSK |
| | | SGTSASLAISGLRSEDEADYYCAAWDDRLNAVVFGGGTKLT |
| | | VLGEQKLISEEDLSGSAAAHHHHHH |
| CIMS (25) | LSADHR | EVQLLESGGGLVQPGGSLRLSCAASGFTFSNYAMSWVRQA |
| | | PGKGLEWVAFIRYDGSNKYYADSVKGRFTISRDNSKNTLYL |
| | | QMNSLRAEDTAVYYCARDAVGGDSYVLDYWGQGTLVTVS |
| | | SGGGGSGGGGSGGGGSQSVLTQPPSASGTPGQRVTISCSG |
| | | SSSNIGSNAVNWYQQLPGTAPKLLIYGNSNRPSGVPDRFSG |
| | | SKSGTSASLAISGLRSEDEADYYCAAWDDSLNGWVFGGGT |
| | | KLTVLGEQKLISEEDLSGSAAAHHHHHH |
| CIMS (27) | SEAHLR | EVQLLESGGGLVQPGGSLRLSCAASGFTFTSYSMSWVRQA |
| | | PGKGLEWVSAIGTGGGTYYADSVKGRFTISRDNSKNTLYLQ |
| | | MNSLRAEDTAVYYCARVNWNDAFDYWGQGTLVTVSSGG |
| | | GGSGGGGSGGGGSQSVLTQPPSASGTPGQRVTISCSGSSS |
| | | NIGNNAVNWYQQLPGTAPKLLIYRNDQRPSGVPDRFSGSK |
| | | SGTSASLAISGLRSEDEADYYCSTWDDSLSGVFFGGGTKLTV |
| | | LGEQKLISEEDLSGSAAAHHHHHH |
| CIMS (28) | SEAHLR | EVQLLESGGGLVQPGGSLRLSCAASGFTFSSYAMSWVRQA |
| | | PGKGLEWVAAIWSDGSNKYYADSVKGRFTISRDNSKNTLYL |
| | | QMNSLRAEDTAVYYCAKVGATDDAFDIWGQGTLVTVSSG |
| | | GGGSGGGGSGGGGSQSVLTQPPSASGTPGQRVTISCSGSS |
| | | SNIGSNTVNWYQQLPGTAPKLLIYGNSNRPSGVPDRFSGSK |
| | | SGISASLAISGLRSEDEADYYCAAWDDSLNGPVFGGGTKLT |
| | | VLGEQKLISEEDLSGSAAAHHHHHH |

SUPPLEMENTARY TABLE S6
Amino acid sequences for scFvs directed against core
biomarkers

| Antibody | Full protein Sequence (VH-linker-VL-tag) |
|---|---|
| MCP-1 (3) | EVQLLESGGGLVQPGGSLRLSCAASGFTFSNYGMHWVRQAPGKGLEWVAVISYDG |
| | SNKYYADSVKGRFTISRDNSKNTLYLQMNSLRAEDTAVYYCAKSHYYDTTSFDYWG |
| | QGTLVTVSSGGGGSGGGGSGGGGSQSVLTQPPSASGTPGQRVTISCSGSSSNIGTN |
| | PVNWYQQLPGTAPKLLIYDNNKRPSGVPDRFSGSKSGTSASLAISGLRSEDEADYYC |
| | AAWDDSLSGVVFGGGTKLTVLGEQKLISEEDLSGSAAAHHHHHH [SEQ ID NO: 1] |
| Procathepsin W | EVQLLESGGGLVQPGGSLRLSCAASGFTFSSYAMSWVRQAPGKGLEWVSSMSASG |
| | GSTYYADSVKGRFTISRDNSKNTLYLQMNSLRAEDTAVYYCARDRGSYGMDVWGQ |
| | GTLVIVSSGGGGSGGGGSGGGGSQSVLTQPPSASGTPGQRVTISCSGSTSNIGSYA |
| | VNWYQQLPGTAPKLLIYGNNNRPSGVPDRFSGSKSGTSASLAISGPRSEDEADYYCA |
| | AWDDSLNGGVFGGGTKLTVLGDYKDDDDKAAAHHHHHH [SEQ ID NO: 2] |
| IL-4 (3) | EVQLLESGGGLVQPGGSLRLSCAASGFTFSNAWMSWVRQAPGKGLEWVSAISGS |
| | GGSTYYADSVKGRFTISRDNSKNTLYLQMNSLRAEDTAVYYCARAIAARPFDYWGQ |
| | GTLVIVSSGGGGSGGGGSGGGGSQSVLTQPPSASGTPGQRVTISCTGATSNIGAGY |
| | DIHWYQQLPGTAPKLLIYSTNNRPSGVPDRFSGSKSGTSASLAISGLRSEDEADYYCA |
| | AWDDSLNGPVFGGGTKLTVLGEQKLISEEDLSGSAAAHHHHHH [SEQ ID NO: 3] |
| Factor B (2) | EVQLLESGGGLVQPGGSLRLSCAASGFTFSSYSMNWVRQAPGKGLEWVAVISYDG |
| | RFIYYSDSVKGRFTISRDNSKNTLYLQMNSLRAEDTAVYYCARSYGGNLAMDVWGQ |
| | GTLVIVSSGGGGSGGGGSGGGGSQSVLTQPPSASGTPGQRVTISCTGSSSNIGAGY |
| | DVHWYQQLPGTAPKLLIYDNNKRPSGVPDRFSGSNSGTSASLAISGLRSEDEADYYC |
| | AAWDDRLNGRVVFGGGTKLTVLGDYKDHDGDYKDHDIDYKDDDDKAAAHHHHH |
| | H [SEQ ID NO: 4] |
| Factor B (3) | EVQLLESGGGLVQPGGSLRLSCAASGFTFSSYSMNWVRQAPGKGLEWVAVISYDG |
| | SNQYYADSVRGRFTISKDNSKNTLYLQMNSLRAEDTAVYYCAREWHYSLDVWGQG |
| | TLVTVSSGGGGSGGGGSGGGGSQSVLTQPPSASGTPGQRVTISCSGSSSNIGSNTV |
| | NWYQQLPGTAPKLLIYRNNQRPSGVPDRFSGSKSGTSASLAISGLRSEDEADYYCAA |
| | WDDSLSVRVFGGGTKLTVLGDYKDHDGDYKDHDIDYKDDDDKAAAHHHHHH |
| | [SEQ ID NO: 5] |
| UPF3B (1) | EVQLLESGGGLVQPGGSLRLSCAASGFTFSDYYMTWIRQAPGKGLEWVSDISWNG |
| | SRTHYADSVKGRFTISRDNSKNTLYLQMNSLRAEDTAVYYCSSHLVYWGQGTLVTV |
| | SSGGGGSGGGGSGGGGSQSVLTQPPSASGTPGQRVTISCTGSSSNIGAGYDVHWY |
| | QQLPGTAPKLLIYDNNKRPSGVPDRFSGSKSGTSASLAISGLRSEDEADYYCQTYDSS |
| | LSGSVVFGGGTKLTVLGDYKDHDGDYKDHDIDYKDDDDKAAAHHHHHH [SEQ ID |
| | NO: 6] |
| HADH2 (3) | EVQLLESGGGLVQPGGSLRLSCAASGFTFGSSYMSWVRQAPGKGLEWVSSISSYGY |
| and/or GSN | YTGYADSVKGRFTISRDNSKNTLYLQMNSLRAEDTAVYYCARSYGSWYFDYWGQG |
| | TLVTVSSGGGGSGGGGSGGGGSDIQMTQSPSSLSASVGDRVTITCRASQSISSYLN |
| | WYQQKPGKAPKLLIYAASSLQSGVPSRFSGSGSGTDFTLTISSLQPEDFATYYCQQGF |
| | VGPSTFGQGTKLEIKRLGDYKDHDGDYKDHDIDYKDDDDKAAAHHHHHH [SEQ |
| | ID NO: 7] |
| PAK-7 (1) | EVQLLESGGGLVQPGGSLRLSCAASGFTFSGYSMSWVRQAPGKGLEWVSSISSSYSS |
| | TYYADSVKGRFTISRDNSKNTLYLQMNSLRAEDTAVYYCARGSFGFDYWGQGTLVT |
| | VSSGGGGSGGGGSGGGGSDIQMTQSPSSLSASVGDRVTITCRASQSISSYLNWYQ |
| | QKPGKAPKLLIYAASSLQSGVPSRFSGSGSGTDFTLTISSLQPEDFATYYCQQYYYGVL |
| | PTFGQGTKLEIKRLGDYKDHDGDYKDHDIDYKDDDDKAAAHHHHHH [SEQ ID |
| | NO: 8] |
| KKCC1-1 (3) | EVQLLESGGGLVQPGGSLRLSCAASGFTFYYSYMYWVRQAPGKGLEWVSAISGSG |
| | GSTYYADSVKGRFTISRDNSKNTLYLQMNSLRAEDTAVYYCARSYTPASYRFDYWG |
| | QGTLVTVSSGGGGSGGGGSGGGGSDIQMTQSPSSLSASVGDRVTITCRASQSISSYL |
| | NWYQQKPGKAPKLLIYAASSLQSGVPSRFSGSGSGTDFTLTISSLQPEDFATYYCQQS |
| | YSTPYTFGQGTKLEIKRLGDYKDHDGDYKDHDIDYKDDDDKAAAHHHHHH [SEQ |
| | ID NO: 9] |
| MAGI1-1 (1) | EVQLLESGGGLVQPGGSLRLSCAASGFTFSYSSMSWVRQAPGKGLEWVSGISGSGY |
| | STYYADSVKGRFTISRDNSKNTLYLQMNSLRAEDTAVYYCARGYSGFDYWGQGTLV |
| | TVSSGGGGSGGGGSGGGGSDIQMTQSPSSLSASVGDRVTITCRASQSISSYLNWYQ |
| | QKPGKAPKLLIYAASSLQSGVPSRFSGSGSGTDFTLTISSLQPEDFATYYCQQYGYATL |
| | PTFGQGTKLEIKRLGDYKDHDGDYKDHDIDYKDDDDKAAAHHHHHH [SEQ ID |
| | NO: 10] |
| OTUB1-1 (2) | EVQLLESGGGLVQPGGSLRLSCAASGFTFSSYAMSWVRQAPGKGLEWVSAISGSG |
| | GSTYYADSVKGRFTISRDNSKNTLYLQMNSLRAEDTAVYYCARYHYYAGFDYWGQG |
| | TLVTVSSGGGGSGGGGSGGGGSDIQMTQSPSSLSASVGDRVTITCRASQSISSYLN |
| | WYQQKPGKAPKLLIYAASSLQSGVPSRFSGSGSGTDFTLTISSLQPEDFATYYCQQSG |
| | SF LPTFGQGTKLEIKRLGDYKDHDGDYKDHDIDYKDDDDKAAAHHHHHH [SEQ ID |
| | NO: 11] |
| FER (1) | EVQLLESGGGLVQPGGSLRLSCAASGFTFGYSYMHWVRQAPGKGLEWVSYISSYG |
| | GYTGYADSVKGRFTISRDNSKNTLYLQMNSLRAEDTAVYYCARSSVDSVVWYGGYI |
| | DYWGQGTLVTVSSGGGGSGGGGSGGGGSDIQMTQSPSSLSASVGDRVTITCRAS |
| | QSISSYLNWYQQKPGKAPKLLIYAASSLQSGVPSRFSGSGSGTDFTLTISSLQPEDFAT |
| | YYCQQSGFNSPHTFGQGTKLEIKRLGDYKDHDGDYKDHDIDYKDDDDKAAAHHHH |
| | HH [SEQ ID NO: 12] |

### REFERENCES

1. Thomas, S. L., Griffiths, C., Smeeth, L., Rooney, C. & Hall, A. J. Burden of mortality associated with autoimmune diseases among females in the United Kingdom. Am J Public Health 100, 2279-2287 (2010).
2. Walsh, S. J. & Rau, L. M. Autoimmune diseases: a leading cause of death among young and middle-aged women in the United States. Am J Public Health 90, 1463-1466 (2000).
3. Manoussakis, M. N. et al. Sjögren's syndrome associated with systemic lupus erythematosus: clinical and laboratory profiles and comparison with primary Sjogren's syndrome. Arthritis and rheumatism 50, 882-891 (2004).
4. Toro-Dominguez, D., Carmona-Saez, P. & Alarcon-Riquelme, M. E. Shared signatures between rheumatoid arthritis, systemic lupus erythematosus and Sjogren's syndrome uncovered through gene expression meta-analysis. Arthritis research & therapy 16, 489 (2014).
5. Alexander, E. L., Hirsch, T. J., Arnett, F. C., Provost, T. T. & Stevens, M. B. Ro(SSA) and La(SSB) antibodies in the clinical spectrum of Sjögren's syndrome. J Rheumatol 9, 239-246 (1982).
6. Falk, R. J. & Jennette, J. C. Anti-neutrophil cytoplasmic autoantibodies with specificity for myeloperoxidase in patients with systemic vasculitis and idiopathic necrotizing and crescentic glomerulonephritis. N Engl J Med 318, 1651-1657 (1988).
7. Rekvig, O. P. Anti-dsDNA antibodies as a classification criterion and a diagnostic marker for systemic lupus erythematosus: critical remarks. Clin Exp Immunol 179, 5-10 (2015).
8. Tervaert, J. W. et al. Autoantibodies against myeloid lysosomal enzymes in crescentic glomerulonephritis. Kidney Int 37, 799-806 (1990).
9. Rasmussen, A. et al. Previous diagnosis of Sjögren's Syndrome as rheumatoid arthritis or systemic lupus erythematosus. Rheumatology (Oxford) 55, 1195-1201 (2016).
10. Haller-Kikkatalo, K. et al. Demographic associations for autoantibodies in disease-free individuals of a European population. Sci Rep 7, 44846 (2017).
11. Tan, E. M. et al. Range of antinuclear antibodies in “healthy” individuals. Arthritis and rheumatism 40, 1601-1611 (1997).
12. Wandstrat, A. E. et al. Autoantibody profiling to identify individuals at risk for systemic lupus erythematosus. Journal of autoimmunity 27, 153-160 (2006).
13. Borrebaeck, C. A. & Wingren, C. Transferring proteomic discoveries into clinical practice. Expert review of proteomics 6, 11-13 (2009).
14. Carlsson, A. et al. Serum Protein Profiling of Systemic Lupus Erythematosus and Systemic Sclerosis Using Recombinant Antibody Microarrays. Mol Cell Proteomics 10 (2011).
15. Eisenberg, R. Why can't we find a new treatment for SLE? Journal of autoimmunity 32, 223-230 (2009).
16. Gibson, D. S. et al. Diagnostic and prognostic biomarker discovery strategies for autoimmune disorders. Journal of proteomics 73, 1045-1060 (2010).
17. Borrebaeck, C. A., Sturfelt, G. & Wingren, C. Recombinant antibody microarray for profiling the serum proteome of SLE. Methods Mol Biol 1134, 67-78 (2014).
18. Petersson, L. et al. Multiplexing of miniaturized planar antibody arrays for serum protein profiling—a biomarker discovery in SLE nephritis. Lab on a chip 14, 1931-1942 (2014).
19. Gladman, D. D., Ibanez, D. & Urowitz, M. B. Systemic lupus erythematosus disease activity index 2000. J Rheumatol 29, 288-291 (2002).
20. Vitali, C. et al. Classification criteria for Sjögren's syndrome: a revised version of the European criteria proposed by the American-European Consensus Group. Annals of the rheumatic diseases 61, 554-558 (2002).
21. Soderlind, E. et al. Recombining germline-derived CDR sequences for creating diverse single-framework antibody libraries. Nature biotechnology 18, 852-856 (2000).
22. Sall, A. et al. Generation and analyses of human synthetic antibody libraries and their application for protein microarrays. Protein engineering, design & selection PEDS 29, 427-437 (2016).
23. Skoog, P. et al. Tumor tissue protein signatures reflect histological grade of breast cancer. PloS one 12, e0179775 (2017).
24. Wu, Y. W. & Wooldridge, P. J. The impact of centering first-level predictors on individual and contextual effects in multilevel data analysis. Nursing research 54, 212-216 (2005).
25. Delfani, P. et al. Technical Advances of the Recombinant Antibody Microarray Technology Platform for Clinical Immunoproteomics. PloS one 11, e0159138 (2016).
26. Carlsson, A. et al. Molecular serum portraits in patients with primary breast cancer predict the development of distant metastases. Proceedings of the National Academy of Sciences of the United States of America 108, 14252-14257 (2011).
27. Carlsson, A. et al. Serum protein profiling of systemic lupus erythematosus and systemic sclerosis using recombinant antibody microarrays. Molecular & cellular proteomics: MCP 10, M110 005033 (2011).
28. Y, B. Y.a.H. Controlling the false discovery rate: a practical and powerful approach to multiple testing. Journal of the Royal Statistical Society Series B 57, 12 (1995).
29. Cooper, G. S., Bynum, M. L. & Somers, E. C. Recent insights in the epidemiology of autoimmune diseases: improved prevalence estimates and understanding of clustering of diseases. Journal of autoimmunity 33, 197-207 (2009).
30. Arbuckle, M. R. et al. Development of autoantibodies before the clinical onset of systemic lupus erythematosus. N Engl J Med 349, 1526-1533 (2003).
31. Eriksson, C. et al. Autoantibodies predate the onset of systemic lupus erythematosus in northern Sweden. Arthritis research & therapy 13, R30 (2011).
32. van Gaalen, F. A. et al. Autoantibodies to cyclic citrullinated peptides predict progression to rheumatoid arthritis in patients with undifferentiated arthritis: a prospective cohort study. Arthritis and rheumatism 50, 709-715 (2004).
33. Visser, H., le Cessie, S., Vos, K., Breedveld, F. C. & Hazes, J. M. How to diagnose rheumatoid arthritis early: a prediction model for persistent (erosive) arthritis. Arthritis and rheumatism 46, 357-365 (2002).
34. Mohan, C. & Assassi, S. Biomarkers in rheumatic diseases: how can they facilitate diagnosis and assessment of disease activity? BMJ 351, h5079 (2015).
35. Ingvarsson, J. et al. Design of recombinant antibody microarrays for serum protein profiling: targeting of complement proteins. Journal of proteome research 6, 3527-3536 (2007).
36. Petersson, L. et al. Miniaturization of multiplexed planar recombinant antibody arrays for serum protein profiling. Bioanalysis 6, 1175-1185 (2014).
37. Chen, M., Daha, M. R. & Kallenberg, C. G. The complement system in systemic autoimmune disease. Journal of autoimmunity 34, J276-286 (2010).
38. Pickart, C. M. Mechanisms underlying ubiquitination. Annu Rev Biochem 70, 503-533 (2001).
39. Weissman, A. M. Themes and variations on ubiquitylation. Nat Rev Mol Cell Biol 2, 169-178 (2001).
40. Espinosa, A. et al. The Sjögren's syndrome-associated autoantigen Ro52 is an E3 ligase that regulates proliferation and cell death. J Immunol 176, 6277-6285 (2006).
41. Feldmann, M. & Maini, R. N. Anti-TNF alpha therapy of rheumatoid arthritis: what have we learned? Annu Rev Immunol 19, 163-196 (2001).
42. Lipsky, P. E. et al. Infliximab and methotrexate in the treatment of rheumatoid arthritis. Anti-Tumor Necrosis Factor Trial in Rheumatoid Arthritis with Concomitant Therapy Study Group. N Engl J Med 343, 1594-1602 (2000).
43. Choy, E. H. et al. Therapeutic benefit of blocking interleukin-6 activity with an anti-interleukin-6 receptor monoclonal antibody in rheumatoid arthritis: a randomized, double-blind, placebo-controlled, dose-escalation trial. Arthritis and rheumatism 46, 3143-3150 (2002).
44. Kaleta, B. Role of osteopontin in systemic lupus erythematosus. Arch Immunol Ther Exp (Warsz) 62, 475-482 (2014).
45. Delfani P, Dexlin Mellby L, Nordstrom M, et al. Technical Advances of the Recombinant Antibody Microarray Technology Platform for Clinical Immunoproteomics. PLoS One (2016); 11: e0159138.
46. Steinhauer C, Wingren C, Hager A C, Borrebaeck C A. Single framework recombinant antibody fragments designed for protein chip applications. Biotechniques (2002); Suppl: 38-45.
47. Wingren C, Borrebaeck C A. Antibody microarray analysis of directly labelled complex proteomes. Curr Opin Biotechnol (2008); 19:55-61.
48. Wingren C, Steinhauer C, Ingvarsson J, Persson E, Larsson K, Borrebaeck C A. Microarrays based on affinity-tagged single-chain Fv antibodies: sensitive detection of analyte in complex proteomes. Proteomics (2005); 5:1281-91.
49. Johnson W E, Li C, Rabinovic A. Adjusting batch effects in microarray expression data using empirical Bayes methods. Biostatistics. (2007); 8(1):118-27.

1. A method for diagnosing or detecting an autoimmune disease in an individual, the method comprising or consisting of the steps of:
a) providing a sample obtained from an individual to be tested; and
b) measuring the presence and/or amount in the test sample of one or more biomarkers selected from the group defined in Table 1(A);
wherein the presence and/or amount in the sample of the one or more biomarker(s) selected from the group defined in Table 1(A) is indicative of an autoimmune disease in the individual.

2. The method according to claim 1 further comprising or consisting of the steps of:
c) providing one or more control samples; and
d) measuring the presence and/or amount in the control sample of the one or more biomarkers measured in step (b);
wherein the individual is identified as having an autoimmune disease by comparing the presence and/or amount in the test sample of the one or more biomarkers measured in step (b) with the presence and/or amount in the control samples.

3. The method according to claim 2 wherein the control samples of step (c) are provided from a healthy individual (negative control) and/or from an individual with an autoimmune disease (positive control).

4. The method according to claim 2 or 3 wherein the control samples of step (c) are provided from an individual with systemic lupus erythematosus (SLE), rheumatoid arthritis (RA), Sjögren's syndrome (SS) or systemic vasculitis (SV).

5. The method according to claim 4 wherein the control samples of step (c) are provided from an individual with systemic lupus erythematosus subtype 1 (SLE-1), systemic lupus erythematosus subtype 2 (SLE-2) or systemic lupus erythematosus subtype 3 (SLE-3).

6. The method according to any one of the preceding claims wherein step (b) comprises or consists of measuring the presence and/or amount in the test sample of two or more of the biomarkers defined in Table 1(A), for example, 3, 4, 5, 6, 7, 8, 9, 10, 11, 12, 13, 14, 15, 16, 17, 18, 19, 20, 21, 22, 23, 24, 25, 26, 27, 28, 29, 30, or 31 of the biomarkers defined in Table 1(A).

7. The method according to any one of the preceding claims wherein step (b) comprises or consists of measuring the presence and/or amount in the test sample of one or more of the biomarkers defined in Table 1(A)i.

8. The method according to any one of the preceding claims wherein step (b) comprises or consists of measuring the presence and/or amount in the test sample of one or more of the biomarkers defined in Table 1(A)ii.

9. The method according to any one of the preceding claims wherein step (b) comprises or consists of measuring the presence and/or amount in the test sample of one or more of the biomarkers defined in Table 1(A)iii.

10. The method according to any one of the preceding claims wherein step (b) comprises or consists of measuring the presence and/or amount in the test sample of biomarkers defined in Table 1(A)i, Table 1(A)ii and/or Table 1(A)iii.

11. The method according to any one of the preceding claims wherein the autoimmune disease is selected from: systemic lupus erythematosus (SLE), rheumatoid arthritis (RA), Sjögren's syndrome (SS) or systemic vasculitis (SV).

12. The method according to any one of the preceding claims wherein the one or more biomarker(s) selected from the group defined in Table 1(A) are biomarkers which are also present in Table 2(A).

13. The method according to any one of the preceding claims wherein the method further comprises measuring the presence and/or amount of one or more of the biomarkers defined in Table 2(A).

14. The method according to any one of the preceding claims, wherein the method further comprises measuring the presence and/or amount of one or more of the biomarkers defined in Table 1(B).

15. The method according to any one of the preceding claims, wherein the method further comprises measuring the presence and/or amount of one or more of the biomarkers defined in Table 1(C).

16. The method according to any one of the preceding claims, wherein the method further comprises measuring the presence and/or amount of one or more of the biomarkers defined in Table 1(D).

17. The method according to any one of the preceding claims, wherein the method further comprises measuring the presence and/or amount of one or more of the biomarkers defined in Table 1(E).

18. A method for diagnosing or detecting systemic lupus erythematosus in an individual comprising or consisting of the steps of:
a) providing one or more sample obtained from an individual with, or suspected of having, an autoimmune disease; and
b) measuring the presence and/or amount in the test sample of one or more biomarker selected from the group defined in Table 1(B);
wherein the presence and/or amount in the one or more test sample of the one or more biomarker(s) selected from the group defined in Table 1(B) is indicative of systemic lupus erythematosus.

19. The method according to claim 18 wherein step (b) comprises or consists of measuring the presence and/or amount in the test sample of one or more of the biomarkers defined in Table 1(B)i.

20. The method according to claim 18 or 19 wherein step (b) comprises or consists of measuring the presence and/or amount in the test sample of one or more of the biomarkers defined in Table 1(B)ii.

21. The method according to any one of claims 18 to 20 wherein step (b) comprises or consists of measuring the presence and/or amount in the test sample of one or more of the biomarkers defined in Table 1(B)iii.

22. The method according to any one of claims 18 to 21 wherein step (b) comprises or consists of measuring the presence and/or amount in the test sample of biomarkers defined in Table 1(B)i, Table 1(B)ii and/or Table 1(B)iii.

23. The method according to any one of claims 18 to 22 wherein the one or more biomarker(s) selected from the group defined in Table 1(B) are biomarkers which are also present in Table 2(B).

24. The method according to any one of claims 18 to 23 wherein the method further comprises measuring the presence and/or amount of one or more of the biomarkers defined in Table 2(B).

25. The method according to any one of claims 18 to 24 further comprising or consisting of the steps of:
c) providing one or more control sample; and
d) measuring the presence and/or amount in the control sample of the one or more biomarkers measured in step (b);
wherein the patient is identified as having systemic lupus erythematosus by comparing the presence and/or amount in the test sample of the one or more biomarkers measured in step (b) with the presence and/or amount in the control samples.

26. A method for diagnosing or detecting rheumatoid arthritis in an individual comprising or consisting of the steps of:
a) providing one or more sample obtained from an individual with, or suspected of having, rheumatoid arthritis; and
b) measuring the presence and/or amount in the test sample of one or more biomarker selected from the group defined in Table 1(C);
wherein the presence and/or amount in the one or more test sample of the one or more biomarker(s) selected from the group defined in Table 1(C) is indicative of rheumatoid arthritis.

27. The method according to claim 26 wherein step (b) comprises or consists of measuring the presence and/or amount in the test sample of one or more of the biomarkers defined in Table 1(C)i.

28. The method according to claim 26 or 27 wherein step (b) comprises or consists of measuring the presence and/or amount in the test sample of one or more of the biomarkers defined in Table 1(C)ii.

29. The method according to any one of claims 26 to 28 wherein step (b) comprises or consists of measuring the presence and/or amount in the test sample of one or more of the biomarkers defined in Table 1(C)iii.

30. The method according to any one of claims 26 to 29 wherein step (b) comprises or consists of measuring the presence and/or amount in the test sample of biomarkers defined in Table 1(C)i, Table 1(C)ii and/or Table 1(C)iii.

31. The method according to any one of claims 26 to 30 wherein the one or more biomarker(s) selected from the group defined in Table 1(C) are biomarkers which are also present in Table 2(C).

32. The method according to any one of claims 26 to 31 wherein the method further comprises measuring the presence and/or amount of one or more of the biomarkers defined in Table 2(C).

33. The method according to any one of claims 26 to 32 further comprising or consisting of the steps of:
c) providing one or more control sample; and
d) measuring the presence and/or amount in the control sample of the one or more biomarkers measured in step (b);
wherein the patient is identified as having rheumatoid arthritis by comparing the presence and/or amount in the test sample of the one or more biomarkers measured in step (b) with the presence and/or amount in the control samples.

34. A method for diagnosing or detecting Sjögren's syndrome in an individual comprising or consisting of the steps of:
a) providing one or more sample obtained from an individual with, or suspected of having, an autoimmune disease; and
b) measuring the presence and/or amount in the test sample of one or more biomarker selected from the group defined in Table 1(D);
wherein the presence and/or amount in the one or more test sample of the one or more biomarker(s) selected from the group defined in Table 1(D) is indicative of Sjögren's syndrome.

35. The method according to claim 34 wherein step (b) comprises or consists of measuring the presence and/or amount in the test sample of one or more of the biomarkers defined in Table 1(D)i.

36. The method according to claim 34 or 35 wherein step (b) comprises or consists of measuring the presence and/or amount in the test sample of one or more of the biomarkers defined in Table 1(D)ii.

37. The method according to any one of claims 34 to 36 wherein step (b) comprises or consists of measuring the presence and/or amount in the test sample of one or more of the biomarkers defined in Table 1(D)iii.

38. The method according to any one of claims 34 to 37 wherein step (b) comprises or consists of measuring the presence and/or amount in the test sample of biomarkers defined in Table 1(D)i, Table 1(D)ii and/or Table 1(D)iii.

39. The method according to any one of claims 34 to 38 wherein the one or more biomarker(s) selected from the group defined in Table 1(D) are biomarkers which are also present in Table 2(D).

40. The method according to any one of claims 34 to 39 wherein the method further comprises measuring the presence and/or amount of one or more of the biomarkers defined in Table 2(D).

41. The method according to any one of claims 34 to 40 further comprising or consisting of the steps of:
c) providing one or more control sample; and
d) measuring the presence and/or amount in the control sample of the one or more biomarkers measured in step (b);
wherein the patient is identified as having Sjögren's syndrome by comparing the presence and/or amount in the test sample of the one or more biomarkers measured in step (b) with the presence and/or amount in the control samples.

42. A method for diagnosing or detecting systemic vasculitis in an individual comprising or consisting of the steps of:
a) providing one or more sample obtained from an individual with, or suspected of having, an autoimmune disease; and
b) measuring the presence and/or amount in the test sample of one or more biomarker selected from the group defined in Table 1(E);
wherein the presence and/or amount in the one or more test sample of the one or more biomarker(s) selected from the group defined in Table 1(E) is indicative of systemic vasculitis.

43. The method according to claim 42 wherein step (b) comprises or consists of measuring the presence and/or amount in the test sample of one or more of the biomarkers defined in Table 1(E)i.

44. The method according to claim 42 or 43 wherein step (b) comprises or consists of measuring the presence and/or amount in the test sample of one or more of the biomarkers defined in Table 1(E)ii.

45. The method according to any one of claims 42 to 44 wherein step (b) comprises or consists of measuring the presence and/or amount in the test sample of one or more of the biomarkers defined in Table 1(E)iii

46. The method according to any one of claims 42 to 45 wherein step (b) comprises or consists of measuring the presence and/or amount in the test sample of biomarkers defined in Table 1(E)i, Table 1(E)ii and/or Table 1(E)iii.

47. The method according to any one of claims 42 to 46 wherein the one or more biomarker(s) selected from the group defined in Table 1(E) are biomarkers which are also present in Table 2(E).

48. The method according to any one of claims 42 to 47 wherein the method further comprises measuring the presence and/or amount of one or more of the biomarkers defined in Table 2(E).

49. The method according to any one of claims 42 to 48 further comprising or consisting of the steps of:
c) providing one or more control sample; and
d) measuring the presence and/or amount in the control sample of the one or more biomarkers measured in step (b);
wherein the patient is identified as having systemic vasculitis by comparing the presence and/or amount in the test sample of the one or more biomarkers measured in step (b) with the presence and/or amount in the control samples.

50. The method according to any one of the preceding claims wherein the method is repeated using a test sample taken from the same individual at a different time period to the previous test sample(s) used.

51. The method according to claim 50 wherein the method is repeated using a test sample taken between 1 day to 104 weeks to the previous test sample(s) used, for example, between 1 week to 100 weeks, 1 week to 90 weeks, 1 week to 80 weeks, 1 week to 70 weeks, 1 week to 60 weeks, 1 week to 50 weeks, 1 week to 40 weeks, 1 week to 30 weeks, 1 week to 20 weeks, 1 week to 10 weeks, 1 week to 9 weeks, 1 week to 8 weeks, 1 week to 7 weeks, 1 week to 6 weeks, 1 week to 5 weeks, 1 week to 4 weeks, 1 week to 3 weeks, or 1 week to 2 weeks.

52. The method according to claim 50 or 51 wherein the method is repeated using a test sample taken every period from the group consisting of: 1 day, 2 days, 3 day, 4 days, 5 days, 6 days, 7 days, 10 days, 2 weeks, 3 weeks, 4 weeks, 5 weeks, 6 weeks, 7 weeks, 8 weeks, 9 weeks, 10 weeks, 15 weeks, 20 weeks, 25 weeks, 30 weeks, 35 weeks, 40 weeks, 45 weeks, 50 weeks, 55 weeks, 60 weeks, 65 weeks, 70 weeks, 75 weeks, 80 weeks, 85 weeks, 90 weeks, 95 weeks, 100 weeks, 104, weeks, 105 weeks, 110 weeks, 115 weeks, 120 weeks, 125 weeks and 130 weeks.

53. The method according to any one of claims 50 to 52 wherein the method is repeated at least once, for example, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 21 times, 22 times, 23, 24 times or 25 times.

54. The method according to any one of the preceding claims wherein a diagnosis in a patient of an autoimmune disease is subsequently confirmed using one or more additional diagnostic tests for the autoimmune disease.

55. The method according to any one of the preceding claims wherein step (b) comprises measuring the expression of the protein or polypeptide of the one or more biomarker(s).

56. The method according to claim 55 wherein step (b) and/or step (d) is performed using one or more first binding agents each capable of binding specifically to a biomarker protein or polypeptide to be measured.

57. The method according to claim 56 wherein the first binding agent is an antibody or a fragment thereof.

58. The method according to claim 57 wherein the antibody or fragment thereof is a recombinant antibody or fragment thereof.

59. The method according to claim 56 or 57 wherein the antibody or fragment thereof is selected from the group consisting of: scFv; Fab; a binding domain of an immunoglobulin molecule.

60. The method according to any one of claims 56 to 59 wherein the first binding agent is immobilised on a surface.

61. The method according to any one of the preceding claims wherein the one or more biomarker(s) in the test sample is labelled with a directly or indirectly detectable moiety.

62. The method according to claim 61 wherein the detectable moiety is selected from the group consisting of: a fluorescent moiety; a luminescent moiety; a chemiluminescent moiety; a radioactive moiety; an enzymatic moiety.

63. The method according to claim 61 or 62 wherein the detectable moiety is biotin.

64. The method according to claim 63 wherein in step (b) and/or step (d) the biotinylated biomarkers are detected using streptavidin labelled with a detectable moiety selected from the group consisting of: a fluorescent moiety; a luminescent moiety; a chemiluminescent moiety; a radioactive moiety; an enzymatic moiety.

65. The method according to claim 64 wherein the detectable moiety is fluorescent moiety (for example an Alexa Fluor dye, e.g. Alexa647).

66. The method according to any one of the preceding claims wherein step (b) and/or step (d) is performed using an array.

67. The method according to claim 66 wherein the array is selected from the group consisting of: macroarray; microarray; nanoarray.

68. The method according to any one of the preceding claims wherein the method comprises:
(i) labelling biomarkers present in the sample with biotin;
(ii) contacting the biotin-labelled proteins with an array comprising a plurality of scFv immobilised at discrete locations on its surface, the scFv having specificity for one or more of the proteins in Table 1(A), Table 1(B), Table 1(C), Table 1(D) and/or Table 1(E);
(iii) contacting the biotin-labelled proteins (immobilised on the scFv) with a streptavidin conjugate comprising a fluorescent dye; and
(iv) detecting the presence of the dye at discrete locations on the array surface
wherein the expression of the dye on the array surface is indicative of the expression of a biomarker from Table 1(A), Table 1(B), Table 1(C), Table 1(D) and/or Table 1(E) in the sample.

69. The method according to any one of the preceding claims wherein step (b) and/or step (d) comprises measuring the expression of a nucleic acid molecule encoding the one or more biomarkers.

70. The method according to claim 69 wherein the nucleic acid molecule is an mRNA molecule.

71. The method according to claim 69 wherein the nucleic acid molecule is a DNA molecule, such as a cDNA or ctDNA molecule.

72. The method according claim 70 or 71, wherein measuring the expression of the one or more biomarker(s) in step (b) and/or step (d) is performed using a method selected from the group consisting of Southern hybridisation, Northern hybridisation, polymerase chain reaction (PCR), reverse transcriptase PCR (RT-PCR), quantitative real-time PCR (qRT-PCR), nanoarray, microarray, macroarray, autoradiography and in situ hybridisation.

73. The method according to any one of claims 70 to 72, wherein measuring the expression of the one or more biomarker(s) in step (b) is determined using a DNA microarray.

74. The method according to any one of claims 70 to 73, wherein measuring the expression of the one or more biomarker(s) in step (b) and/or step (d) is performed using one or more binding moieties, each individually capable of binding selectively to a nucleic acid molecule encoding one of the biomarkers identified in Table 1(A), Table 1(B), Table 1(C), Table 1(D) and/or Table 1(E).

75. The method according to claim 74, wherein the one or more binding moieties each comprise or consist of a nucleic acid molecule.

76. The method according to claim 74 or 75, wherein the one or more binding moieties each comprise or consist of DNA.

77. The method according to any one of claims 74 to 76 wherein the one or more binding moieties are 5 to 100 nucleotides in length, for example 15 to 35 nucleotides in length.

78. The method according to any one of claims 74 to 77 wherein the binding moiety comprises a detectable moiety.

79. The method according to claim 78 wherein the detectable moiety is selected from the group consisting of: a fluorescent moiety; a luminescent moiety; a chemiluminescent moiety; a radioactive moiety (for example, a radioactive atom); or
an enzymatic moiety.

80. The method according to claim 79 wherein the detectable moiety comprises or consists of a radioactive atom.

81. The method according to claim 80 wherein the radioactive atom is selected from the group consisting of technetium-99m, iodine-123, iodine-125, iodine-131, indium-111, fluorine-19, carbon-13, nitrogen-15, oxygen-17, phosphorus-32, sulphur-35, deuterium, tritium, rhenium-186, rhenium-188 and yttrium-90.

82. The method according to claim 79 wherein the detectable moiety of the binding moiety is a fluorescent moiety.

83. The method according to any one of the preceding claims wherein the sample provided in step (a) and/or step (c) is selected from the group consisting of unfractionated blood, plasma, serum, tissue fluid, milk, bile, synovial fluid, and urine.

84. The method according to claim 83, wherein the sample provided in step (a) and/or step (c) is selected from the group consisting of unfractionated blood, plasma and serum.

85. The method according to claim 83 or 84, wherein the sample provided in step (a) and/or step (c) is serum.

86. The method according to any one of the preceding claims wherein the predictive accuracy of the method, as determined by an ROC AUC value, is at least 0.50, for example at least 0.55, 0.60, 0.65, 0.70, 0.75, 0.80, 0.85, 0.90, 0.95, 0.96, 0.97, 0.98 or at least 0.99.

87. The method according to claim 86 wherein the predictive accuracy of the method, as determined by an ROC AUC value, is at least 0.70.

88. The method according to any one of the preceding claims wherein, in the event that the individual is diagnosed with an autoimmune disease, the method comprises an additional step of administering to the individual a therapy for said autoimmune disease.

89. The method according to claim 88 wherein the autoimmune disease therapy is selected from the group consisting of: Nonsteroidal anti-inflammatory drugs (NSAID) such as Ibuprofen and Naproxen; Immune-suppressing drugs such as Corticosteroids; synthetic DMARDs (such as Methotrexate, cyclophosphoamide); and Biologicals (such as TNF-inhibitors, IL-inhibitors); and combinations thereof.

90. An array for diagnosing or detecting an autoimmune disease in an individual comprising one or agents suitable for measuring the presence and/or amount of one or more biomarkers selected from the group defined in Table 1(A), Table 1(B), Table 1(C), Table 1(D), and/or Table 1(E).

91. An array according to claim 78 comprising one or more binding agents as defined in any one of claims 56 to 65 or 74 to 82.

92. An array according to claim 78 or 79 wherein the one or more binding agents are collectively capable of binding to all of the proteins defined in Table 1(A), Table 1(B), Table 1(C), Table 1(D), and/or Table 1(E).

93. Use of one or more biomarkers selected from the group defined in Table 1(A), Table 1(B), Table 1(C), Table 1(D), or Table 1(E) as a biomarker for diagnosing or detecting an autoimmune disease in an individual, optionally wherein the autoimmune disease is selected from systemic lupus erythematosus (SLE), rheumatoid arthritis (RA), Sjögren's syndrome (SS) or systemic vasculitis (SV).

94. The use according to claim 81 wherein all of the biomarkers defined in Table 1(A), Table 1(B), Table 1(C), Table 1(D), and/or Table 1(E) are used as a biomarker for diagnosing or detecting an autoimmune disease in an individual, optionally wherein the autoimmune disease is selected from systemic lupus erythematosus (SLE), rheumatoid arthritis (RA), Sjögren's syndrome (SS) or systemic vasculitis (SV).

95. A kit for diagnosing or detecting an autoimmune disease in an individual comprising:
i) one or more first binding agents as defined in any one of claims 56 to 65 or 74 to 82,
ii) (optionally) instructions for performing the method as defined in any one of claims 1 to 89.

96. A method of treating an autoimmune disease in an individual comprising the steps of:
(a) diagnosing an individual with an autoimmune disease using a method according to any one of claims 1 to 89; and
(b) providing the individual with a therapy to treating said autoimmune disease.

97. A method or use substantially as described herein.

98. An array or kit substantially as described herein.
